(19) United States
(12) Patent Application Publication
(10) Pub. No.: US 2025/0163397 A1
(43) Pub. Date: May 22, 2025

(54) LACTASE ENZYMES WITH IMPROVED PROPERTIES

(71) Applicant: Kerry Group Services International Ltd, Tralee (IE)

(72) Inventors: Hans Raj, Hoersholm (DK); Pernille Smith, Broenshoej (DK); Thomas Eckhardt, Birkeroed (DK); Vojislav Vojinovic, Graested (DK); Charlotte Elisabeth Grüner Schöller, Virum (DK); Johannes Maarten van den Brink, Herlev (DK)

(73) Assignee: Kerry Group Services International Ltd, Tralee (IE)

(21) Appl. No.: 19/029,745

(22) Filed: Jan. 17, 2025

Related U.S. Application Data
(63) Continuation of application No. 17/986,618, filed on Nov. 14, 2022, now Pat. No. 12,215,367.
Application No. 17/986,618 is a continuation of application No. 16/604,129, filed on Oct. 9, 2019, now Pat. No. 11,525,129.

(30) Foreign Application Priority Data
Apr. 11, 2017 (EP) 17166021.0

(51) Int. Cl. C12N 9/38; A23L 29/00

(52) U.S. Cl. CPC C12N 9/2471; A23L 29/06

(57) ABSTRACT

The present invention relates to new improved peptide or dimeric peptides exhibiting beta- galactosidase enzyme activity as well as improved methods for reducing the lactose content in compositions, such as dairy products.

### CROSS REFERENCE TO RELATED APPLICATIONS

The present application is a continuation of U.S. application Ser. No. 17/986,618, filed Nov. 14, 2022 (granting as U.S. Pat. No. 12,215,367), which is a continuation of U.S. application Ser. No. 16/604,129, filed Oct. 9, 2019 (now U.S. Pat. No. 11,525,129), which is the U.S. National Stage of International Application No. PCT/EP2018/059250, filed Apr. 11, 2018, which claims priority to European Patent Application No. 17166021.0, filed Apr. 11, 2017.

### SEQUENCE LISTING

The instant application contains a Sequence Listing which has been submitted electronically in XML format and is hereby incorporated by reference in its entirety. Said XML copy, created on Jan. 16, 2023, is named “030427-0380_SL.xml” and is 70,779 bytes in size.

### FIELD OF THE INVENTION

The present invention relates to new improved peptide or dimeric peptides exhibiting beta-galactosidase enzyme activity as well as improved methods for reducing the lactose content in compositions, such as dairy products.

### BACKGROUND OF THE INVENTION

In order to grow on milk, lactose hydrolysis is a good way for lactic acid bacteria to obtain glucose and galactose as carbon source. Lactase (beta-galactosidase; EC 3.2.1.23) is the enzyme that performs the hydrolysis step of the milk sugar lactose into monosaccharides. The commercial use of lactase is to break down lactose in dairy products. Lactose intolerant people have difficulties to digest dairy products with high lactose levels. It is estimated that about 70% of the world's population has a limited ability to digest lactose. Accordingly, there is a growing demand for dairy food products that contain no or only low levels of lactose.

Lactases have been isolated from a large variety of organisms, including microorganisms like Kluyveromyces and Bacillus. Kluyveromyces, especially K. fragilis and K. lactis, and other fungi such as those of the genera Candida, Torula and Torulopsis, are a common source of fungal lactases, whereas B. coagulans and B. circulans are well known sources for bacterial lactases. Several commercial lactase preparations derived from these organisms are available such as Lactozym® (available from Novozymes, Denmark), HA-Lactase (available from Chr. Hansen, Denmark) and Maxilact® (available from DSM, the Netherlands), all from K. lactis. All these lactases are so-called neutral lactases having a pH optimum between pH 6 and pH 8, as well as a temperature optimum around 37° C. When such lactases are used in the production of, e.g. low-lactose yoghurt, the enzyme treatment will either have to be done in a separate step before fermentation or rather high enzyme dosages have to be used because their activity will drop as the pH decreases during fermentation. Also, these lactases are not suitable for hydrolysis of lactose in milk performed at high temperature, which would in some cases be beneficial in order to keep the microbial count low and thus ensure high milk quality. Furthermore, the known lactases would not be suitable for use in a desired process for the production of ultra-heat treated (UHT) milk, wherein enzymes were added prior to the UHT treatment.

WO2010092057 and WO0104276 relates to cold-active beta-galactosidases. WO07110619 relates to beta-galactosidase with high transgalactosylating activity, whereas WO2009071539 relates to beta-galactosidase with lower transgalactosylating activity.

### OBJECT OF THE INVENTION

It is an object of embodiments of the invention to provide beta-galactosidases with properties that enable the production of improved lactose-free or low-lactose products.

It is a further object of embodiments of the invention to provide beta-galactosidases with properties that enable the improved, such as easier, faster, more reliable or less expensive production methods for the lowering of lactose in a product, such as lactose-free or low-lactose products.

### SUMMARY OF THE INVENTION

The present inventor(s) have identified beta-galactosidases with properties not previously described that enable the production of improved lactose-free or low-lactose products as well as enabling improved production methods for such lactose-free or low-lactose products. In particular these beta-galactosidases have been shown to be very stable with relatively high activity at a very broad range of both temperatures as well as pH values. They are also useable at specific temperatures, such as at high temperatures and pH values not normally seen with these enzymes. First of all, this enables to the use of beta-galactosidases at specific pH values and temperatures that were not known to be possible. It also enables the use of the same specific enzyme in several different applications, which is highly requested in the industry.

So, in a first aspect the present invention relates to a peptide exhibiting beta-galactosidase enzyme activity, which has an amino acid sequence represented by SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, or an amino acid sequence thereof having not more than 1, 2, 3, 4, 5, 6, 7, 8, 9, 10, 11, 12, 13, 14, 15, 16, 17, 18, 19, 20, 21, or 22 amino acid substitutions, additions or deletions.

In a second aspect the present invention relates to a dimeric peptide exhibiting beta-galactosidase enzyme activity, which dimeric peptide consist of two peptides having an amino acid sequence represented by SEQ ID NO: 2 and 3; 5 and 6; 20 and 21; 23 and 24; 26 and 27; or 28 and 29, or enzymatically active fragments thereof, or an amino acid sequence of any one thereof having not more than 1, 2, 3, 4, 5, 6, 7, 8, 9, 10, 11, 12, 13, 14, 15, 16, 17, 18, 19, 20, 21, or 22 amino acid substitutions, additions or deletions.

In a third aspect the present invention relates to a nucleotide sequence which encodes a peptide or dimeric peptide exhibiting beta-galactosidase enzyme activity according to the invention.

In a further aspect the present invention relates to a host cell comprising a nucleotide sequence which encodes a peptide or dimeric peptide exhibiting beta-galactosidase enzyme activity according to the invention.

In a further aspect the present invention relates to a method for producing a peptide or dimeric peptide exhibiting beta-galactosidase enzyme activity according to the invention, which method comprises the expression of a vector containing a nucleotide sequence according to the invention in a suitable host cell; and purifying said peptide or dimeric peptide from the expression products of said host cell.

In a further aspect the present invention relates to a method for reducing the lactose content in a composition containing lactose, such as in a dairy products, comprising the step of contacting said composition with a peptide or dimeric peptide exhibiting beta-galactosidase enzyme activity, which peptide has an amino acid sequence represented by SEQ ID NO:1-33; or which dimeric peptide consist of two peptides having an amino acid sequence represented by SEQ ID NO: 2 and 3, 5 and 6, 20 and 21, 23 and 24, 26 and 27, or 28 and 29; or a sequence with at least 80% sequence identity to any one of said sequences; or a host cell expressing any one of said peptides, at a pH ranging from 3-10 and at a temperature ranging from 0° C.-140° C.

In a further aspect the present invention relates to the use of a peptide or dimeric peptide exhibiting beta-galactosidase enzyme activity, which peptide has an amino acid sequence represented by SEQ ID NO:1-33, or which dimeric peptide consist of two peptides having an amino acid sequence represented by SEQ ID NO: 2 and 3, 5 and 6, 20 and 21, 23 and 24, 26 and 27, or 28 and 29; or a sequence with at least 80% sequence identity to any one of said sequences; or a host cell expressing any one of said peptides for producing a dairy product with a reduced lactose content.

In some embodiments this composition containing lactose or this dairy product is selected from the group consisting of lactose-free milk, low-lactose milk, yoghurt, including unpasteurized as well as pre and post-pasteurized yoghurt, cheese, fermented milk products, dietary supplement and probiotic dietary products. In some other embodiments this host cell is any one selected from a bacteria of the genus Bifidobacterium, such as Bifidobacterium adolescentis, Bifidobacterium bifidum, Bifidobacterium breve, Bifidobacterium catenulatum, Bifidobacterium longum or from the genus Lactobacillus, such as L. sakei, L. amylovorus, L. delbrueckii subsp. bulgaricus, L. delbrueckii subsp. lactis, L. delbrueckii subsp. Indicus, L. crispatus, L. reuteri, L. helveticus or from Streptococcus thermophilus. In some other embodiments the lactose concentration is reduced to less than about 1%, such as to less than about 0.1% or lower, such as to less than about 0.01%.

In a further aspect the present invention relates to a method for producing a dairy product the method comprising the steps of:
a) providing a milk-based substrate comprising lactose;
b) adding a peptide or dimeric peptide exhibiting beta-galactosidase activity, which peptide has an amino acid sequence represented by SEQ ID NO:1-33; or which dimeric peptide consist of two peptides having an amino acid sequence represented by SEQ ID NO: 2 and 3, 5 and 6, 20 and 21, 23 and 24, 26 and 27, or 28 and 29; or a sequence with at least 80% sequence identity to any one of said sequences to said milk-based substrate comprising lactose; and
c) treating said milk-based substrate with said peptide or dimeric peptide exhibiting beta-galactosidase activity.

In a further aspect the present invention relates to a dairy product prepared by a method according to the invention.

In a further aspect the present invention relates to a food product, such as a dairy product comprising a peptide or dimeric peptide exhibiting beta-galactosidase enzyme activity, which peptide has an amino acid sequence represented by SEQ ID NO:1-33, or which dimeric peptide consist of two peptides having an amino acid sequence represented by SEQ ID NO: 2 and 3, 5 and 6, 20 and 21, 23 and 24, 26 and 27, or 28 and 29; or a sequence with at least 80% sequence identity to any one of said sequences.

In a further aspect the present invention relates to a food product, such as a dairy product comprising a host cell expressing a peptide or dimeric peptide exhibiting beta-galactosidase enzyme activity, which peptide has an amino acid sequence represented by SEQ ID NO:1-33, or which dimeric peptide consist of two peptides having an amino acid sequence represented by SEQ ID NO: 2 and 3, 5 and 6, 20 and 21, 23 and 24, 26 and 27, or 28 and 29; or a sequence with at least 80% sequence identity to any one of said sequences. In some specific embodiments such a food product is selected from beverages, infant foods, cereals, bread, biscuits, confectionary, cakes, food supplements, dietary supplements, probiotic comestible products, prebiotic comestible products, animal feeds, poultry feeds and medicaments, or a dairy product selected from the group consisting of lactose-free milk, low-lactose milk, dried milk powder, baby milks, yoghurt, ice cream, cheese, fermented milk products, dietary supplement and probiotic dietary products.

### LEGENDS TO THE FIGURE

FIG. 1. The specific activity of the purified enzymes determined at pH 6.7 at 37° C. with lactose as substrate, described SUAL-1, discussed in example 6. The measured standard deviation at the given condition was less than 6%.

FIG. 2. The specific activity of the purified enzymes determined at pH 6.7 at 37° C. in presence of galactose, described as SUAG, discussed in example 7. The measured standard deviation at the given condition was less than 15%.

FIG. 3. The specific activity of the purified enzymes determined at pH 6.7 at 4° C. with lactose as substrate, described as SUAL-2, discussed in example 8. The measured standard deviation at the given condition was less than 5%.

FIG. 4. The specific activity of the purified enzymes determined at pH 6.7 at 43° C. with lactose as substrate, described as SUAL-3, discussed in example 9. The measured standard deviation at the given condition was less than 5%.

FIG. 5. The specific activity of the purified enzymes determined at pH 5.5 at 4° C. with lactose as substrate, described as SUAL-4, discussed in example 10. The measured standard deviation at the given condition was less than 5%.

FIG. 6. The specific activity of the purified enzymes determined at pH 5.5 at 37° C. with lactose as substrate, described as SUAL-5, discussed in example 11. The measured standard deviation at the given condition was less than 5%.

FIG. 7. The specific activity of the purified enzymes determined at pH 5.5 at 43° C. with lactose as substrate, described as SUAL-6, discussed in example 12. The measured standard deviation at the given condition was less than 5%.

FIG. 8. The specific activity of the purified enzymes determined at pH 4.5 at 4° C. with lactose as substrate, described as SUAL-7, discussed in example 13. The measured standard deviation at the given condition was less than 5%.

FIG. 9. The specific activity of the purified enzymes determined at pH 4.5 at 37° C. with lactose as substrate, described as SUAL-8, discussed in example 14. The measured standard deviation at the given condition was less than 5%.

FIG. 10. The specific activity of the purified enzymes determined at pH 4.5 at 43° C. with lactose as substrate, described as SUAL-9, discussed in example 15. The measured standard deviation at the given condition was less than 5%.

FIG. 11. The percentage residual lactose in the pasteurized milk, after the treatment with a fixed amount of the enzyme, after 24 hr at 5° C. determined using HPLC.

FIG. 12. The percentage residual lactose in the UHT milk, after the treatment with a fixed amount of the enzyme, after 24 hr at 25° C. determined using HPLC.

FIG. 13. The percentage residual activity of the purified enzymes at elevated temperatures, determined using lactose as substrate. The activity at pH 6.7 at 37° C. was considered as 100%.

FIG. 14. The specific activity of the purified enzymes determined at pH 6.7 at 4° C., 37° C. and 43° C. The measured specific activity is described as μmol glucose formed per minute per mg of enzyme. The galactose inhibition method is described in example 7 and calculated based on the μmol glucose formed per minute per mg of enzyme.

FIG. 15. The specific activity of the purified enzymes determined at pH 5.5 at 4° C., 37° C. and 43° C. The measured specific activity is described as μmol glucose formed per minute per mg of enzyme.

FIG. 16. The specific activity of the purified enzymes determined at pH 4.5 at 4° C., 37° C. and 43° C. The measured specific activity is described as μmol glucose formed per minute per mg of enzyme.

### DETAILED DISCLOSURE OF THE INVENTION

The present inventors have found that certain peptides and dimeric peptides exhibiting beta-galactosidase enzyme activity are surprisingly stabile at many different physical conditions giving a relatively high activity outside of the ranges normally seen to be optimal for this class of enzymes.

Accordingly, these by the present inventors identified enzymes have a relatively high activity around 4° C. or 5° C. and may thus be used for lactose hydrolysis in the production of e.g. fresh milk. Moreover, the enzymes have also a relatively high activity in the range of 10° C.-25° C. and the exact same enzymes may thus be used for lactose hydrolysis in UHT milk. This feasibility of the enzymes even at broad ranges of temperatures is highly relevant since milk may be stored at room/ambient temperature which may be different in different parts of the world, also depending on the seasons. For the UHT treatment, the temperature is typically either around 135° C. or around 140° C. It is highly wanted that the enzymes may have activity in the range of a temperature up to 140° C. so that the enzyme may be added to raw milk before the UHT step. In the current practices the enzyme is added after the UHT step because the enzymes known in the art has a significant decrease in functional activity, such as to a value below measurable activity following the high heat treatment step. Also the milk is stored at room temperature which may vary significantly in different parts of the world.

Also these novel improved peptides exhibiting beta-galactosidase enzyme activity have been found to have activity in the temperature range normally used for pasteurization. Accordingly, these enzymes may be added to raw milk prior to pasteurization. It is to be understood that the enzymes known in the art has a significant decrease in functional activity, such as to a value below measurable activity following a pasteurization step.

A further advantage of these novel improved peptides exhibiting beta-galactosidase enzyme activity is that they have a relatively low degree of galactose inhibition. The lower galactose inhibition of these novel enzymes is highly relevant for applications wherein very low lactose concentrations are desired.

In terms of applicability for fermented products it is highly advantageous that the enzymes as described herein have a high beta-galactosidase enzymatic activity at a relatively broad temperature range of between 4° C.-43° C., such as around 37° C., where fermentation would normally be optimal, but also that this activity of the beta-galactosidase enzyme is present at low pH, such as down to 4.5, or down to 4.0, or down to 3.5, or even down to pH 3.

In summary, it has been found by the present inventors that some peptides exhibiting beta-galactosidase enzyme activity is active over wide range of temperature, active over wide range of pH, has a general high hydrolytic activity without side activities, that these peptides have no or little galactose inhibition, such as less than 60%, and that they are stable over long-term storage.

The beta-galactosidase activity may be determined by measuring the amount of released glucose after incubation with lactose at set conditions. Released glucose can be detected by a coloring reaction.

### Definitions

The term “milk”, as used herein and in the context of the present invention, is to be understood as the lacteal secretion obtained by milking any mammal, such as cow, sheep, goats, buffalo or camel.

The term “composition containing lactose” as used herein refers to any composition, such as any liquid that contain lactose in significant measurable degree, such as a lactose content higher than 0.002% (0.002 g/100 ml). Encompassed within this term are milk and milk-based substrates.

The term “milk-based substrate”, in the context of the present invention, may be any raw and/or processed milk material. Useful milk-based substrates include, but are not limited to solutions/suspensions of any milk or milk like products comprising lactose, such as whole or low fat milk, skim milk, buttermilk, low-lactose milk, reconstituted milk powder, condensed milk, solutions of dried milk, UHT milk, whey, whey permeate, acid whey, cream, fermented milk products, such as yoghurt, cheese, dietary supplement and probiotic dietary products. Typically the term milk-based substrate refers to a raw or processed milk material that is processed further in order to produce a dairy product.

The term “pasteurization” as used herein refers to the process of reducing or eliminating the presence of live organisms, such as microorganisms in a milk-based substrate. Preferably, pasteurization is attained by maintaining a specified temperature for a specified period of time. The specified temperature is usually attained by heating. The temperature and duration may be selected in order to kill or inactivate certain bacteria, such as harmful bacteria, and/or to inactivate enzymes in the milk. A rapid cooling step may follow.

The term “dairy product” as used herein may be any food product wherein one of the major constituents is milk-based. Usually the major constituent is milk-based and in some embodiments, the major constituent is a milk-based substrate which has been treated with an enzyme having beta-galactosidase activity according to a method of the present invention.

A dairy product according to the invention may be, e.g., skim milk, low fat milk, whole milk, cream, UHT milk, milk having an extended shelf life, a fermented milk product, cheese, yoghurt, butter, dairy spread, butter milk, acidified milk drink, sour cream, whey based drink, ice cream, condensed milk, dulce de leche or a flavored milk drink.

A dairy product may additionally comprise non-milk components, e.g. vegetable components such as, e.g., vegetable oil, vegetable protein, and/or vegetable carbohydrates. Dairy products may also comprise further additives such as, e.g., enzymes, flavoring agents, microbial cultures such as probiotic cultures, salts, sweeteners, sugars, acids, fruit, fruit prep, fruit juices, or any other component known in the art as a component of, or additive to, a dairy product.

The terms “fermented dairy product” or “fermented milk product” as used herein is to be understood as any dairy product wherein any type of fermentation forms part of the production process. Examples of fermented dairy products are products like yoghurt, buttermilk, creme fraiche, quark and fromage frais. A fermented dairy product may be produced by or include steps of any method known in the art.

The term “fermentation” as used herein refers to the conversion of carbohydrates into alcohols or acids through the action of a microorganism. In some embodiments fermentation according to the present invention comprises the conversion of lactose to lactic acid. In the context of the present invention, “microorganism” may include any bacterium or fungus being able to ferment the milk substrate.

The term “increased beta-galactosidase enzyme activity” as used herein refers to a relatively higher specific activity of a beta-galactosidase enzyme in comparison to a reference sequence.

The term “peptide exhibiting beta-galactosidase enzyme activity” as used herein refers to any peptide, which has enzymatic activity to catalyze the hydrolysis of the disaccharide lactose into its component monosaccharides glucose and galactose. This peptide may also be referred to as a lactase or simply a beta-galactosidase (EC: 3.2.1.23).

The terms “peptide” and “oligopeptide” as used in the context of this present application are considered synonymous (as is commonly recognized) and each term can be used interchangeably as the context requires to indicate a chain of at least two amino acids coupled by peptidyl linkages. The word “polypeptide” is used herein for chains containing more than ten amino acid residues. All peptide and polypeptide formulas or sequences herein are written from left to right and in the direction from amino terminus to carboxy terminus. “Proteins” as used herein refers to peptide sequences as they are produced by some host organism and may include posttranslational modification, such as added glycans.

The terms “amino acid” or “amino acid sequence,” as used herein, refer to an oligopeptide, peptide, polypeptide, or protein sequence, or a fragment of any of these, and to naturally occurring or synthetic molecules. In this context, “fragment” refer to fragments of a peptide exhibiting beta-galactosidase enzyme activity, which retain some enzymatic activity. Where “amino acid sequence” is recited herein to refer to an amino acid sequence of a naturally occurring protein molecule, “amino acid sequence” and like terms are not meant to limit the amino acid sequence to the complete native amino acid sequence associated with the recited peptide molecule.

Exemplary peptides of the invention also include fragments of at least about 50,100,150, 200, 250, 300, 350, 400, 450, 500, 550, 600, 650, 700, 750, 800 or more residues in length, or over the full length of an enzyme. Accordingly a “peptide fragment” or “enzymatically active fragment” of the invention are fragments that retain at least some functional enzymatic activity.

Typically a peptide fragment of the invention will still contain the functional catalytic domain or other essential active sites of the peptide exhibiting beta-galactosidase enzyme activity. Other domains may be deleted.

Typically, the specific beta-galactosidase enzyme activity will be measured and indicated as μmol of glucose formed/min/mg of enzyme used. This specific value however will vary depending on conditions applied, such as temperature, and pH. Accordingly, values for beta-galactosidase enzyme activity may also be referred to as relative to a reference known enzyme, such as the beta-galactosidase enzyme defined by SEQ ID NO:34 OR SEQ ID NO:35.

Alternatively the specific beta-galactosidase enzyme activity may be measured and indicated as μM of glucose formed per second per μM of enzyme used. This specific value however will vary depending on conditions applied, such as temperature, and pH. ½

Unless otherwise stated the term “Sequence identity” for amino acids as used herein refers to the sequence identity calculated as (n_{ref}−n_{dif})·100/n_{ref}, wherein n_{dif} is the total number of non-identical residues in the two sequences when aligned and wherein n_{ref} is the number of residues in one of the sequences.

In some embodiments the sequence identity is determined by conventional methods, e.g., Smith and Waterman, 1981, Adv. Appl. Math. 2:482, by the search for similarity method of Pearson & Lipman, 1988, Proc. Natl. Acad. Sci. USA 85:2444, using the CLUSTAL W algorithm of Thompson et al., 1994, Nucleic Acids Res 22:467380, by computerized implementations of these algorithms (GAP, BESTFIT, FASTA, and TFASTA in the Wisconsin Genetics Software Package, Genetics Computer Group). The BLAST algorithm (Altschul et al., 1990, Mol. Biol. 215:403-10) for which software may be obtained through the National Center for Biotechnology Information www.ncbi.nlm.nih.gov/) may also be used. When using any of the aforementioned algorithms, the default parameters for “Window” length, gap penalty, etc., are used.

A peptide with a specific amino acid sequence as described herein may vary from a reference peptide sequence by any of amino acid substitutions, additions/insertions, or deletions.

Some embodiments according to the present invention refers to the use of a peptide with an amino acid sequence represented by SEQ ID NO:1-33 or a sequence with at least 80% sequence identity to any one of said sequences. In some embodiments this sequence identity may be at least about 81%, 82%, 83%, 84%, 85%, 86%, 87%, 88%, 89%, 90%, 91%, 92%, 93%, 94%, 95%, 96%, 97%, 98%, or 99%, such as a peptide with not more than 1, 2, 3, 4, 5, 6, 7, 8, 9, 10, 11, 12, 13, 14, 15, 16, 17, 18, 19, 20, 21, or 22 amino acid substitutions, additions or deletions as compared to any one reference amino acid sequence represented by SEQ ID NO:1-33. The invention also features biologically active fragments of the peptides according to the invention. Biologically active fragments of a peptide of the invention include peptides comprising amino acid sequences sufficiently identical to or derived from the amino acid sequence of peptide of the invention which include fewer amino acids than the full length protein but which exhibit a substantial part of the biological activity of the corresponding full-length peptide. Typically, biologically active fragments comprise a domain or motif with at least one activity of a variant protein of the invention. A biologically active fragment of a peptide of the invention can be a peptide which is, for example, 100, 200, 300, 400, 500, 600, 700, 800, 900, 1000 or more amino acids in length.

The term “host cell”, as used herein, includes any cell type which is susceptible to transformation, transfection, transduction, and the like with a nucleic acid construct or expression vector comprising a polynucleotide encoding the peptides of the present invention. A host cell may be the cell type, where a specific enzyme is derived from or it may be an alternative cell type susceptible to the production of a specific enzyme. The term includes both wild type and attenuated strains.

Suitable host cell may be any bacteria including lactic acid within the order “Lactobacillies” which includes Lactococcus spp., Streptococcus spp., Lactobacillus spp., Leuconostoc spp., Pseudoleuconostoc spp., Pediococcus spp., Brevibacterium spp., Enterococcus spp. and Propionibacterium spp. Also included are lactic acid producing bacteria belonging to the group of anaerobic bacteria, bifidobacteria, i.e. Bifidobacterium spp., which are frequently used as food cultures alone or in combination with lactic acid bacteria. Also included within this definition are Lactococcus lactis, Lactococcus lactis subsp. cremoris, Leuconostoc mesenteroides subsp. cremoris, Pseudoleuconostoc mesenteroides subsp. cremoris, Pediococcus pentosaceus, Lactococcus lactis subsp. lactis biovar. diacetylactis, Lactobacillus casei subsp. casei and Lactobacillus paracasei subsp. Paracasei and thermophilic lactic acid bacterial species include as examples Streptococcus thermophilus, Enterococcus faecium, Lactobacillus delbrueckii subsp. lactis, Lactobacillus helveticus, Lactobacillus delbrueckii subsp. bulgaricus and Lactobacillus acidophilus. Other specific bacteria within this definition includes bacteria of the family Bifidobacteriaceae, such as from the genus Bifidobacterium, such as from a strain of bifidobacterium animalis or bifidobacterium longum, bifidobacterium adolescentis, bifidobacterium bifodum, bifidobacterium breve, bifidobacterium catenulatum, bifidobacterium infantus or from the genus Lactobacillus, such as L. sakei, L. amylovorus, L. delbrueckii subsp. Lactis, and L. helveticus.

Also included within this definition of host cells include strain of Agaricus, e.g. A. bisporus; Ascovaginospora; Aspergillus, e.g. A. niger, A. awamori, A. foetidus, A. japonicus, A. oryzae; Candida; Chaetomium; Chaetotomastia; Dictyostelium, e.g. D. discoideum; Kluveromyces, e.g. K. fragilis, K. lactis; Mucor, e.g. M. javanicus, M. mucedo, M. subtilissimus; Neurospora, e.g. N. crassa; Rhizomucor, e.g. R. pusillus; Rhizopus, e.g. R. arrhizus, R. japonicus, R. stolonifer; Sclerotinia, e.g. S. libertiana; Torula; Torulopsis; Trichophyton, e.g. T. rubrum; Whetzelinia, e.g. W. sclerotiorum; Bacillus, e.g. B. coagulans, B. circulans, B. megaterium, B. novalis, B. subtilis, B. pumilus, B. stearothermophilus, B. thuringiensis; Bifidobacterium, e.g. B. Iongum, B. bifidum, B. animalis; Chryseobacterium; Citrobacter, e.g. C. freundii; Clostridium, e.g. C. perfringens; Diplodia, e.g. D. gossypina; Enterobacter, e.g. E. aerogenes, E. cloacae Edwardsiella, E. tarda; Erwinia, e.g. E. herbicola; Escherichia, e.g. E. coli; Klebsiella, e.g. K. pneumoniae; Miriococcum; Myrothesium; Mucor; Neurospora, e.g. N. crassa; Proteus, e.g. P. vulgaris; Providencia, e.g. P. stuartii; Pycnoporus, e.g. Pycnoporus cinnabarinus, Pycnoporus sanguineus; Ruminococcus, e.g. R. torques; Salmonella, e.g. S. typhimurium; Serratia, e.g. S. liquefasciens, S. marcescens; Shigella, e.g. S. flexneri; Streptomyces, e.g. S. antibioticus, S. castaneoglobisporus, S. violeceoruber; Trametes; Trichoderma, e.g. T. reesei, T. viride; Yersinia, e.g. Y. enterocolitica.

### Specific Embodiments of the Invention

As described above the present invention relates to a peptide exhibiting beta-galactosidase enzyme activity, which has an amino acid sequence represented by SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, or an amino acid sequence thereof having not more than 1, 2, 3, 4, 5, 6, 7, 8, 9, 10, 11, 12, 13, 14, 15, 16, 17, 18, 19, 20, 21, or 22 amino acid substitutions, additions or deletions.

Accordingly, in one embodiment the present invention relates to a peptide exhibiting beta-galactosidase enzyme activity, which has an amino acid sequence represented by SEQ ID NO: 1, or an amino acid sequence thereof having not more than 1, 2, 3, 4, 5, 6, 7, 8, 9, 10, 11, 12, 13, 14, 15, 16, 17, 18, 19, 20, 21, or 22 amino acid substitutions, additions or deletions. In one embodiment the present invention relates to a peptide exhibiting beta-galactosidase enzyme activity, which has an amino acid sequence represented by SEQ ID NO: 2, or an amino acid sequence thereof having not more than 1, 2, 3, 4, 5, 6, 7, 8, 9, 10, 11, 12, 13, 14, 15, 16, 17, 18, 19, 20, 21, or 22 amino acid substitutions, additions or deletions. In one embodiment the present invention relates to a peptide exhibiting beta-galactosidase enzyme activity, which has an amino acid sequence represented by SEQ ID NO: 3, or an amino acid sequence thereof having not more than 1, 2, 3, 4, 5, 6, 7, 8, 9, 10, 11, 12, 13, 14, 15, 16, 17, 18, 19, 20, 21, or 22 amino acid substitutions, additions or deletions. In one embodiment the present invention relates to a peptide exhibiting beta-galactosidase enzyme activity, which has an amino acid sequence represented by SEQ ID NO: 4, or an amino acid sequence thereof having not more than 1, 2, 3, 4, 5, 6, 7, 8, 9, 10, 11, 12, 13, 14, 15, 16, 17, 18, 19, 20, 21, or 22 amino acid substitutions, additions or deletions. In one embodiment the present invention relates to a peptide exhibiting beta-galactosidase enzyme activity, which has an amino acid sequence represented by SEQ ID NO: 5, or an amino acid sequence thereof having not more than 1, 2, 3, 4, 5, 6, 7, 8, 9, 10, 11, 12, 13, 14, 15, 16, 17, 18, 19, 20, 21, or 22 amino acid substitutions, additions or deletions. In one embodiment the present invention relates to a peptide exhibiting beta-galactosidase enzyme activity, which has an amino acid sequence represented by SEQ ID NO: 6, or an amino acid sequence thereof having not more than 1, 2, 3, 4, 5, 6, 7, 8, 9, 10, 11, 12, 13, 14, 15, 16, 17, 18, 19, 20, 21, or 22 amino acid substitutions, additions or deletions. In one embodiment the present invention relates to a peptide exhibiting beta-galactosidase enzyme activity, which has an amino acid sequence represented by SEQ ID NO: 7, or an amino acid sequence thereof having not more than 1, 2, 3, 4, 5, 6, 7, 8, 9, 10, 11, 12, 13, 14, 15, 16, 17, 18, 19, 20, 21, or 22 amino acid substitutions, additions or deletions. In one embodiment the present invention relates to a peptide exhibiting beta-galactosidase enzyme activity, which has an amino acid sequence represented by SEQ ID NO: 8, or an amino acid sequence thereof having not more than 1, 2, 3, 4, 5, 6, 7, 8, 9, 10, 11, 12, 13, 14, 15, 16, 17, 18, 19, 20, 21, or 22 amino acid substitutions, additions or deletions. In one embodiment the present invention relates to a peptide exhibiting beta-galactosidase enzyme activity, which has an amino acid sequence represented by SEQ ID NO: 9, or an amino acid sequence thereof having not more than 1, 2, 3, 4, 5, 6, 7, 8, 9, 10, 11, 12, 13, 14, 15, 16, 17, 18, 19, 20, 21, or 22 amino acid substitutions, additions or deletions. In one embodiment the present invention relates to a peptide exhibiting beta-galactosidase enzyme activity, which has an amino acid sequence represented by SEQ ID NO: 10, or an amino acid sequence thereof having not more than 1, 2, 3, 4, 5, 6, 7, 8, 9, 10, 11, 12, 13, 14, 15, 16, 17, 18, 19, 20, 21, or 22 amino acid substitutions, additions or deletions. In one embodiment the present invention relates to a peptide exhibiting beta-galactosidase enzyme activity, which has an amino acid sequence represented by SEQ ID NO: 11, or an amino acid sequence thereof having not more than 1, 2, 3, 4, 5, 6, 7, 8, 9, 10, 11, 12, 13, 14, 15, 16, 17, 18, 19, 20, 21, or 22 amino acid substitutions, additions or deletions. In one embodiment the present invention relates to a peptide exhibiting beta-galactosidase enzyme activity, which has an amino acid sequence represented by SEQ ID NO: 12, or an amino acid sequence thereof having not more than 1, 2, 3, 4, 5, 6, 7, 8, 9, 10, 11, 12, 13, 14, 15, 16, 17, 18, 19, 20, 21, or 22 amino acid substitutions, additions or deletions. In one embodiment the present invention relates to a peptide exhibiting beta-galactosidase enzyme activity, which has an amino acid sequence represented by SEQ ID NO: 13, or an amino acid sequence thereof having not more than 1, 2, 3, 4, 5, 6, 7, 8, 9, 10, 11, 12, 13, 14, 15, 16, 17, 18, 19, 20, 21, or 22 amino acid substitutions, additions or deletions. In one embodiment the present invention relates to a peptide exhibiting beta-galactosidase enzyme activity, which has an amino acid sequence represented by SEQ ID NO: 14, or an amino acid sequence thereof having not more than 1, 2, 3, 4, 5, 6, 7, 8, 9, 10, 11, 12, 13, 14, 15, 16, 17, 18, 19, 20, 21, or 22 amino acid substitutions, additions or deletions. In one embodiment the present invention relates to a peptide exhibiting beta-galactosidase enzyme activity, which has an amino acid sequence represented by SEQ ID NO: 15, or an amino acid sequence thereof having not more than 1, 2, 3, 4, 5, 6, 7, 8, 9, 10, 11, 12, 13, 14, 15, 16, 17, 18, 19, 20, 21, or 22 amino acid substitutions, additions or deletions. In one embodiment the present invention relates to a peptide exhibiting beta-galactosidase enzyme activity, which has an amino acid sequence represented by SEQ ID NO: 16, or an amino acid sequence thereof having not more than 1, 2, 3, 4, 5, 6, 7, 8, 9, 10, 11, 12, 13, 14, 15, 16, 17, 18, 19, 20, 21, or 22 amino acid substitutions, additions or deletions. In one embodiment the present invention relates to a peptide exhibiting beta-galactosidase enzyme activity, which has an amino acid sequence represented by SEQ ID NO: 17, or an amino acid sequence thereof having not more than 1, 2, 3, 4, 5, 6, 7, 8, 9, 10, 11, 12, 13, 14, 15, 16, 17, 18, 19, 20, 21, or 22 amino acid substitutions, additions or deletions. In one embodiment the present invention relates to a peptide exhibiting beta-galactosidase enzyme activity, which has an amino acid sequence represented by SEQ ID NO: 18, or an amino acid sequence thereof having not more than 1, 2, 3, 4, 5, 6, 7, 8, 9, 10, 11, 12, 13, 14, 15, 16, 17, 18, 19, 20, 21, or 22 amino acid substitutions, additions or deletions. In one embodiment the present invention relates to a peptide exhibiting beta-galactosidase enzyme activity, which has an amino acid sequence represented by SEQ ID NO: 19, or an amino acid sequence thereof having not more than 1, 2, 3, 4, 5, 6, 7, 8, 9, 10, 11, 12, 13, 14, 15, 16, 17, 18, 19, 20, 21, or 22 amino acid substitutions, additions or deletions. In one embodiment the present invention relates to a peptide exhibiting beta-galactosidase enzyme activity, which has an amino acid sequence represented by SEQ ID NO: 20, or an amino acid sequence thereof having not more than 1, 2, 3, 4, 5, 6, 7, 8, 9, 10, 11, 12, 13, 14, 15, 16, 17, 18, 19, 20, 21, or 22 amino acid substitutions, additions or deletions. In one embodiment the present invention relates to a peptide exhibiting beta-galactosidase enzyme activity, which has an amino acid sequence represented by SEQ ID NO: 21, or an amino acid sequence thereof having not more than 1, 2, 3, 4, 5, 6, 7, 8, 9, 10, 11, 12, 13, 14, 15, 16, 17, 18, 19, 20, 21, or 22 amino acid substitutions, additions or deletions. In one embodiment the present invention relates to a peptide exhibiting beta-galactosidase enzyme activity, which has an amino acid sequence represented by SEQ ID NO: 22, or an amino acid sequence thereof having not more than 1, 2, 3, 4, 5, 6, 7, 8, 9, 10, 11, 12, 13, 14, 15, 16, 17, 18, 19, 20, 21, or 22 amino acid substitutions, additions or deletions. In one embodiment the present invention relates to a peptide exhibiting beta-galactosidase enzyme activity, which has an amino acid sequence represented by SEQ ID NO: 23, or an amino acid sequence thereof having not more than 1, 2, 3, 4, 5, 6, 7, 8, 9, 10, 11, 12, 13, 14, 15, 16, 17, 18, 19, 20, 21, or 22 amino acid substitutions, additions or deletions. In one embodiment the present invention relates to a peptide exhibiting beta-galactosidase enzyme activity, which has an amino acid sequence represented by SEQ ID NO: 24, or an amino acid sequence thereof having not more than 1, 2, 3, 4, 5, 6, 7, 8, 9, 10, 11, 12, 13, 14, 15, 16, 17, 18, 19, 20, 21, or 22 amino acid substitutions, additions or deletions. In one embodiment the present invention relates to a peptide exhibiting beta-galactosidase enzyme activity, which has an amino acid sequence represented by SEQ ID NO: 25, or an amino acid sequence thereof having not more than 1, 2, 3, 4, 5, 6, 7, 8, 9, 10, 11, 12, 13, 14, 15, 16, 17, 18, 19, 20, 21, or 22 amino acid substitutions, additions or deletions. In one embodiment the present invention relates to a peptide exhibiting beta-galactosidase enzyme activity, which has an amino acid sequence represented by SEQ ID NO: 26, or an amino acid sequence thereof having not more than 1, 2, 3, 4, 5, 6, 7, 8, 9, 10, 11, 12, 13, 14, 15, 16, 17, 18, 19, 20, 21, or 22 amino acid substitutions, additions or deletions. In one embodiment the present invention relates to a peptide exhibiting beta-galactosidase enzyme activity, which has an amino acid sequence represented by SEQ ID NO: 27, or an amino acid sequence thereof having not more than 1, 2, 3, 4, 5, 6, 7, 8, 9, 10, 11, 12, 13, 14, 15, 16, 17, 18, 19, 20, 21, or 22 amino acid substitutions, additions or deletions. In one embodiment the present invention relates to a peptide exhibiting beta-galactosidase enzyme activity, which has an amino acid sequence represented by SEQ ID NO: 28, or an amino acid sequence thereof having not more than 1, 2, 3, 4, 5, 6, 7, 8, 9, 10, 11, 12, 13, 14, 15, 16, 17, 18, 19, 20, 21, or 22 amino acid substitutions, additions or deletions. In one embodiment the present invention relates to a peptide exhibiting beta-galactosidase enzyme activity, which has an amino acid sequence represented by SEQ ID NO: 29, or an amino acid sequence thereof having not more than 1, 2, 3, 4, 5, 6, 7, 8, 9, 10, 11, 12, 13, 14, 15, 16, 17, 18, 19, 20, 21, or 22 amino acid substitutions, additions or deletions. In one embodiment the present invention relates to a peptide exhibiting beta-galactosidase enzyme activity, which has an amino acid sequence represented by SEQ ID NO: 30, or an amino acid sequence thereof having not more than 1, 2, 3, 4, 5, 6, 7, 8, 9, 10, 11, 12, 13, 14, 15, 16, 17, 18, 19, 20, 21, or 22 amino acid substitutions, additions or deletions. In one embodiment the present invention relates to a peptide exhibiting beta-galactosidase enzyme activity, which has an amino acid sequence represented by SEQ ID NO: 31, or an amino acid sequence thereof having not more than 1, 2, 3, 4, 5, 6, 7, 8, 9, 10, 11, 12, 13, 14, 15, 16, 17, 18, 19, 20, 21, or 22 amino acid substitutions, additions or deletions. In one embodiment the present invention relates to a peptide exhibiting beta-galactosidase enzyme activity, which has an amino acid sequence represented by SEQ ID NO: 32, or an amino acid sequence thereof having not more than 1, 2, 3, 4, 5, 6, 7, 8, 9, 10, 11, 12, 13, 14, 15, 16, 17, 18, 19, 20, 21, or 22 amino acid substitutions, additions or deletions. In one embodiment the present invention relates to a peptide exhibiting beta-galactosidase enzyme activity, which has an amino acid sequence represented by SEQ ID NO: 33, or an amino acid sequence thereof having not more than 1, 2, 3, 4, 5, 6, 7, 8, 9, 10, 11, 12, 13, 14, 15, 16, 17, 18, 19, 20, 21, or 22 amino acid substitutions, additions or deletions.

In some embodiments the peptide according to the invention has a beta-galactosidase activity measured as μM of glucose formed per second per μM of enzyme under conditions as given in example 8 described herein at a temperature of about 4° C. and a pH of 6.7, which activity is exceeding the activity of a beta-galactosidase enzyme defined by SEQ ID NO:34 OR SEQ ID NO:35 by at least about 20%, such as at least about 30%, such as at least about 40%, such as at least about 50%, such as at least about 60%, such as at least about 70%, such as at least about 80%, such as at least about 90%, such as at least about 100%, such as at least about 200%, such as at least about 300%, such as at least about 400%, such as at least about 500%.

In some embodiments the peptide according to the invention has a beta-galactosidase activity measured as μM of glucose formed per second per μM of enzyme at a temperature of about 4° C. and a pH of 6.7, which activity is exceeding the activity of a beta-galactosidase enzyme defined by SEQ ID NO:34 OR SEQ ID NO:35 by at least about 20%, such as at least about 30%, such as at least about 40%, such as at least about 50%, such as at least about 60%, such as at least about 70%, such as at least about 80%, such as at least about 90%, such as at least about 100%, such as at least about 200%, such as at least about 300%, such as at least about 400%, such as at least about 500%. In some embodiments the peptide according to the invention has a beta-galactosidase activity measured as μM of glucose formed per second per μM of enzyme at a temperature of about 4° C. and a pH of 6.7, which activity is higher than about 2, such as higher than about 4, such as higher than about 6, such as higher than about 8, such as higher than about 10, such as higher than about 12, such as higher than about 14, such as higher than about 16 μM of glucose formed per second per μM of enzyme.

In some embodiments the peptide according to the invention has a beta-galactosidase activity measured as μM of glucose formed per second per μM of enzyme under conditions as given in example 10 described herein at a temperature of about 4° C. and a pH of 5.5, which activity is exceeding the activity of a beta-galactosidase enzyme defined by SEQ ID NO:34 OR SEQ ID NO:35 by at least about 20%, such as at least about 30%, such as at least about 40%, such as at least about 50%, such as at least about 60%, such as at least about 70%, such as at least about 80%, such as at least about 90%, such as at least about 100%, such as at least about 200%, such as at least about 300%, such as at least about 400%, such as at least about 500%.

In some embodiments the peptide according to the invention has a beta-galactosidase activity measured as μM of glucose formed per second per μM of enzyme at a temperature of about 4° C. and a pH of 5.5, which activity is exceeding the activity of a beta-galactosidase enzyme defined by SEQ ID NO:34 OR SEQ ID NO:35 by at least about 20%, such as at least about 30%, such as at least about 40%, such as at least about 50%, such as at least about 60%, such as at least about 70%, such as at least about 80%, such as at least about 90%, such as at least about 100%, such as at least about 200%, such as at least about 300%, such as at least about 400%, such as at least about 500%. In some embodiments the peptide according to the invention has a beta-galactosidase activity measured as μM of glucose formed per second per μM of enzyme at a temperature of about 4° C. and a pH of 5.5, which activity is higher than about 1, such as higher than about 2, such as higher than about 3, such as higher than about 4, such as higher than about 5, such as higher than about 6, such as higher than about 7, such as higher than about 8 μM of glucose formed per second per μM of enzyme.

In some embodiments the peptide according to the invention has a beta-galactosidase activity measured as μM of glucose formed per second per μM of enzyme under conditions as given in example 13 described herein at a temperature of about 4° C. and a pH of 4.5, which activity is exceeding the activity of a beta-galactosidase enzyme defined by SEQ ID NO:34 OR SEQ ID NO:35 by at least about 20%, such as at least about 30%, such as at least about 40%, such as at least about 50%, such as at least about 60%, such as at least about 70%, such as at least about 80%, such as at least about 90%, such as at least about 100%, such as at least about 200%, such as at least about 300%, such as at least about 400%, such as at least about 500%.

In some embodiments the peptide according to the invention has a beta-galactosidase activity measured as μM of glucose formed per second per μM of enzyme at a temperature of about 4° C. and a pH of 4.5, which activity is exceeding the activity of a beta-galactosidase enzyme defined by SEQ ID NO:34 OR SEQ ID NO:35 by at least about 20%, such as at least about 30%, such as at least about 40%, such as at least about 50%, such as at least about 60%, such as at least about 70%, such as at least about 80%, such as at least about 90%, such as at least about 100%, such as at least about 200%, such as at least about 300%, such as at least about 400%, such as at least about 500%. In some embodiments the peptide according to the invention has a beta-galactosidase activity measured as μM of glucose formed per second per μM of enzyme at a temperature of about 4° C. and a pH of 4.5, which activity is higher than about 0.5, such as higher than about 1.0, such as higher than about 1.5, such as higher than about 2.0, such as higher than about 2.5, such as higher than about 3.0, such as higher than about 3.5, such as higher than about 4.0 μM of glucose formed per second per μM of enzyme.

In some embodiments the peptide according to the invention has a beta-galactosidase activity measured as μM of glucose formed per second per μM of enzyme under conditions as given in example 9 described herein at a temperature of about 43° C. and a pH of 6.7, which activity is exceeding the activity of a beta-galactosidase enzyme defined by SEQ ID NO:34 OR SEQ ID NO:35 by at least about 20%, such as at least about 30%, such as at least about 40%, such as at least about 50%, such as at least about 60%, such as at least about 70%, such as at least about 80%, such as at least about 90%, such as at least about 100%, such as at least about 200%, such as at least about 300%, such as at least about 400%, such as at least about 500%.

In some embodiments the peptide according to the invention has a beta-galactosidase activity measured as μM of glucose formed per second per μM of enzyme at a temperature of about 43° C. and a pH of 6.7, which activity is exceeding the activity of a beta-galactosidase enzyme defined by SEQ ID NO:34 OR SEQ ID NO:35 by at least about 20%, such as at least about 30%, such as at least about 40%, such as at least about 50%, such as at least about 60%, such as at least about 70%, such as at least about 80%, such as at least about 90%, such as at least about 100%, such as at least about 200%, such as at least about 300%, such as at least about 400%, such as at least about 500%. In some embodiments the peptide according to the invention has a beta-galactosidase activity measured as μM of glucose formed per second per μM of enzyme at a temperature of about 43° C. and a pH of 6.7, which activity is higher than about 10, such as higher than about 20, such as higher than about 40, such as higher than about 60, such as higher than about 80, such as higher than about 100, such as higher than about 120, such as higher than about 140, such as higher than about 160 μM of glucose formed per second per μM of enzyme.

In some embodiments the peptide according to the invention has a beta-galactosidase activity measured as μM of glucose formed per second per μM of enzyme under conditions as given in example 12 described herein at a temperature of about 43° C. and a pH of 5.5, which activity is exceeding the activity of a beta-galactosidase enzyme defined by SEQ ID NO:34 OR SEQ ID NO: 35 by at least about 20%, such as at least about 30%, such as at least about 40%, such as at least about 50%, such as at least about 60%, such as at least about 70%, such as at least about 80%, such as at least about 90%, such as at least about 100%, such as at least about 200%, such as at least about 300%, such as at least about 400%, such as at least about 500%.

In some embodiments the peptide according to the invention has a beta-galactosidase activity measured as μM of glucose formed per second per μM of enzyme at a temperature of about 43° C. and a pH of 5.5, which activity is exceeding the activity of a beta-galactosidase enzyme defined by SEQ ID NO:34 OR SEQ ID NO:35 by at least about 20%, such as at least about 30%, such as at least about 40%, such as at least about 50%, such as at least about 60%, such as at least about 70%, such as at least about 80%, such as at least about 90%, such as at least about 100%, such as at least about 200%, such as at least about 300%, such as at least about 400%, such as at least about 500%. In some embodiments the peptide according to the invention has a beta-galactosidase activity measured as μM of glucose formed per second per μM of enzyme at a temperature of about 43° C. and a pH of 5.5, which activity is higher than about 5, such as higher than about 10, such as higher than about 15, such as higher than about 20, such as higher than about 25, such as higher than about 30, such as higher than about 35, such as higher than about 40, such as higher than about 45, such as higher than about 50, such as higher than about 55, such as higher than about 60 μM of glucose formed per second per μM of enzyme.

In some embodiments the peptide according to the invention has a beta-galactosidase activity measured as μM of glucose formed per second per μM of enzyme under conditions as given in example 15 described herein at a temperature of about 43° C. and a pH of 4.5, which activity is exceeding the activity of a beta-galactosidase enzyme defined by SEQ ID NO:34 OR SEQ ID NO:35 by at least about 20%, such as at least about 30%, such as at least about 40%, such as at least about 50%, such as at least about 60%, such as at least about 70%, such as at least about 80%, such as at least about 90%, such as at least about 100%, such as at least about 200%, such as at least about 300%, such as at least about 400%, such as at least about 500%.

In some embodiments the peptide according to the invention has a beta-galactosidase activity measured as μM of glucose formed per second per μM of enzyme at a temperature of about 43° C. and a pH of 4.5, which activity is exceeding the activity of a beta-galactosidase enzyme defined by SEQ ID NO:34 OR SEQ ID NO:35 by at least about 20%, such as at least about 30%, such as at least about 40%, such as at least about 50%, such as at least about 60%, such as at least about 70%, such as at least about 80%, such as at least about 90%, such as at least about 100%, such as at least about 200%, such as at least about 300%, such as at least about 400%, such as at least about 500%. In some embodiments the peptide according to the invention has a beta-galactosidase activity measured as μM of glucose formed per second per μM of enzyme at a temperature of about 43° C. and a pH of 4.5, which activity is higher than about 1, such as higher than about 2, such as higher than about 3, such as higher than about 4, such as higher than about 5, such as higher than about 6, such as higher than about 7, such as higher than about 8, such as higher than about 9, such as higher than about 10, such as higher than about 11, such as higher than about 12 μM of glucose formed per second per μM of enzyme.

In some embodiments the peptide according to the invention has a beta-galactosidase activity measured as μM of glucose formed per second per μM of enzyme under conditions as given in example 6 described herein at a temperature of about 37° C. and a pH of 6.7, which activity is exceeding the activity of a beta-galactosidase enzyme defined by SEQ ID NO:34 OR SEQ ID NO:35 by at least about 20%, such as at least about 30%, such as at least about 40%, such as at least about 50%, such as at least about 60%, such as at least about 70%, such as at least about 80%, such as at least about 90%, such as at least about 100%, such as at least about 200%, such as at least about 300%, such as at least about 400%, such as at least about 500%.

In some embodiments the peptide according to the invention has a beta-galactosidase activity measured as μM of glucose formed per second per μM of enzyme at a temperature of about 37° C. and a pH of 6.7, which activity is exceeding the activity of a beta-galactosidase enzyme defined by SEQ ID NO:34 OR SEQ ID NO:35 by at least about 20%, such as at least about 30%, such as at least about 40%, such as at least about 50%, such as at least about 60%, such as at least about 70%, such as at least about 80%, such as at least about 90%, such as at least about 100%, such as at least about 200%, such as at least about 300%, such as at least about 400%, such as at least about 500%. In some embodiments the peptide according to the invention has a beta-galactosidase activity measured as μM of glucose formed per second per μM of enzyme at a temperature of about 37° C. and a pH of 6.7, which activity is higher than about 10, such as higher than about 20, such as higher than about 30, such as higher than about 40, such as higher than about 50, such as higher than about 60, such as higher than about 70, such as higher than about 80, such as higher than about 90, such as higher than about 100, such as higher than about 110, such as higher than about 120, such as higher than about 130 μM of glucose formed per second per μM of enzyme.

In some embodiments the peptide according to the invention has a beta-galactosidase activity measured as μM of glucose formed per second per μM of enzyme under conditions as given in example 11 described herein at a temperature of about 37° C. and a pH of 5.5, which activity is exceeding the activity of a beta-galactosidase enzyme defined by SEQ ID NO:34 OR SEQ ID NO:35 by at least about 20%, such as at least about 30%, such as at least about 40%, such as at least about 50%, such as at least about 60%, such as at least about 70%, such as at least about 80%, such as at least about 90%, such as at least about 100%, such as at least about 200%, such as at least about 300%, such as at least about 400%, such as at least about 500%.

In some embodiments the peptide according to the invention has a beta-galactosidase activity measured as μM of glucose formed per second per μM of enzyme at a temperature of about 37° C. and a pH of 5.5, which activity is exceeding the activity of a beta-galactosidase enzyme defined by SEQ ID NO:34 OR SEQ ID NO:35 by at least about 20%, such as at least about 30%, such as at least about 40%, such as at least about 50%, such as at least about 60%, such as at least about 70%, such as at least about 80%, such as at least about 90%, such as at least about 100%, such as at least about 200%, such as at least about 300%, such as at least about 400%, such as at least about 500%. In some embodiments the peptide according to the invention has a beta-galactosidase activity measured as μM of glucose formed per second per μM of enzyme at a temperature of about 37° C. and a pH of 5.5, which activity is higher than about 5, such as higher than about 10, such as higher than about 15, such as higher than about 20, such as higher than about 25, such as higher than about 30, such as higher than about 35, such as higher than about 40, such as higher than about 45, such as higher than about 50, such as higher than about 55, such as higher than about 60 μM of glucose formed per second per μM of enzyme.

In some embodiments the peptide according to the invention has a beta-galactosidase activity measured as μM of glucose formed per second per μM of enzyme under conditions as given in example 14 described herein at a temperature of about 37° C. and a pH of 4.5, which activity is exceeding the activity of a beta-galactosidase enzyme defined by SEQ ID NO:34 OR SEQ ID NO:35 by at least about 20%, such as at least about 30%, such as at least about 40%, such as at least about 50%, such as at least about 60%, such as at least about 70%, such as at least about 80%, such as at least about 90%, such as at least about 100%, such as at least about 200%, such as at least about 300%, such as at least about 400%, such as at least about 500%.

In some embodiments the peptide according to the invention has a beta-galactosidase activity measured as μM of glucose formed per second per μM of enzyme at a temperature of about 37° C. and a pH of 4.5, which activity is exceeding the activity of a beta-galactosidase enzyme defined by SEQ ID NO:34 OR SEQ ID NO:35 by at least about 20%, such as at least about 30%, such as at least about 40%, such as at least about 50%, such as at least about 60%, such as at least about 70%, such as at least about 80%, such as at least about 90%, such as at least about 100%, such as at least about 200%, such as at least about 300%, such as at least about 400%, such as at least about 500%. In some embodiments the peptide according to the invention has a beta-galactosidase activity measured as μM of glucose formed per second per μM of enzyme at a temperature of about 37° C. and a pH of 4.5, which activity is higher than about 1, such as higher than about 2, such as higher than about 3, such as higher than about 4, such as higher than about 5, such as higher than about 6, such as higher than about 7, such as higher than about 8, such as higher than about 9, such as higher than about 10, such as higher than about 11, such as higher than about 12, such as higher than about 13, such as higher than about 14, such as higher than about 15, such as higher than about 16, such as higher than about 17, such as higher than about 18 μM of glucose formed per second per μM of enzyme.

In some embodiments the peptide according to the invention is derived from a bacteria of the genus Bifidobacterium, such as Bifidobacterium adolescentis, Bifidobacterium bifidum, Bifidobacterium breve, Bifidobacterium catenulatum, Bifidobacterium longum or from the genus Lactobacillus, such as L. sakei, L. amylovorus, L. delbrueckii subsp. bulgaricus, L. delbrueckii subsp. lactis, L. delbrueckii subsp. Indicus, L. crispatus, L. reuteri, L. helveticus or from Streptococcus thermophilus.

In some embodiments the peptide according to the invention exhibit a galactose inhibition less than 60%, such as less than 55%, such as less than 50%, such as less than about 45%, such as less than about 40%.

As described above at part of the present invention relates to a method for producing a dairy product the method comprising the steps of
a) providing a milk-based substrate comprising lactose;
b) adding an peptide exhibiting beta-galactosidase activity and having an amino acid sequence represented by SEQ ID NO:1-33 or a sequence with at least 80% sequence identity to any one of said sequences to said milk-based substrate comprising lactose; and
c) treating said milk-based substrate with said peptide exhibiting beta-galactosidase activity.

In some embodiments according to the present invention this peptide is derived from any one bacteria of the genus Bifidobacterium, such as Bifidobacterium adolescentis, Bifidobacterium bifidum, Bifidobacterium breve, Bifidobacterium catenulatum, Bifidobacterium longum or from the genus Lactobacillus, such as L. sakei, L. amylovorus, L. delbrueckii subsp. bulgaricus, L. delbrueckii subsp. lactis, L. delbrueckii subsp. Indicus, L. crispatus, L. reuteri, L. helveticus or from Streptococcus thermophilus.

In some embodiments according to the present invention step c) takes place at a pH within a range of 3-10, such as within a range of 3-9, such as within a range of 3-8, such as within a range of 3-7, such as within a range of 3-6, such as within a range of 3-5, such as within a range of 3-4, such as within a range of 4-10, such as within a range of 4-9, such as within a range of 4-8, such as within a range of 4-7, such as within a range of 4-6, such as within a range of 4-5, such as within a range of 5-10, such as within a range of 5-9, such as within a range of 5-8, such as within a range of 5-7, such as within a range of 5-6, such as within a range of 6-10, such as within a range of 6-9, such as within a range of 6-8, such as within a range of 6-7.

In some embodiments according to the present invention step c) or a part of step c) takes place at a temperature of not more than about 25° C., such as not more than about 20° C., such as not more than about 18° C., such as not more than about 16° C., such as not more than about 14° C., such as not more than about 12° C., such as not more than about 10° C., such as not more than about 8° C., such as not more than about 7° C., such as not more than about 6° C., such as not more than about 5° C., such as not more than about 4° C., such as not more than about 3° C., such as not more than about 2° C.

In some embodiments according to the present invention step c) or a part of step c) takes place at a temperature of at least about 25° C., such as at least about 30° C., such as at least about 35° C., such as at least about 40° C., such as at least about 45° C., such as at least about 50° C., such as at least about 55° C., such as at least about 60° C., such as at least about 65° C., such as at least about 70° C., such as at least about 75° C., such as at least about 80° C., such as at least about 85° C., such as at least about 90° C., such as at least about 95° C., such as at least about 100° C., such as at least about 110° C., such as at least about 120° C., such as at least about 130° C., such as at least about 120° C., such as at least about 130° C., such as at least about 135° C., such as at least about 140° C.

In some embodiments according to the present invention the dairy product is selected from the group consisting of lactose-free milk, low-lactose milk, yoghurt, cheese, fermented milk products, dietary supplement and probiotic dietary products.

In some embodiments according to the present invention the milk-based substrate is selected from fresh milk or raw milk obtained directly from a step of pasteurization, milk obtained directly after a step of ultra-heat treatment (UHT), or milk obtained directly after a step of fermentation.

In some embodiments according to the present invention the galactose inhibition of the peptide used is less than 60%, such as less than 55%, such as less than 50%, such as less than about 45%, such as less than about 40%.

In some embodiments according to the present invention the dairy product is fermented milk product and said step b) is performed during or prior to fermentation.

In some embodiments according to the present invention the method does not require the addition of further enzyme after fermentation.

In some embodiments according to the present invention the dairy product is fermented milk product and said step b) is performed immediately following fermentation.

In some embodiments according to the present invention the dairy product is fresh milk and said step b) is performed prior to, in conjunction with, or immediately following a step of pasteurization.

In some embodiments according to the present invention the dairy product is ultra-heat treatment (UHT) milk and said step b) is performed prior to, in conjunction with, or immediately following a step of ultra-heat treatment.

In some embodiments according to the present invention step c) is started at a temperature of between 40° C. and 100° C., such as at a temperature of between 50° C. and 100° C. such as at a temperature of between 60° C. and 100° C., such as at a temperature of between 70° C. and 100° C., such as at a temperature of between 80° C. and 100° C., such as at a temperature of between 40° C. and 90° C., such as at a temperature of between 40° C. and 80° C., such as at a temperature of between 40° C. and 70° C., such as at a temperature of between 40° C. and 60° C., such as at a temperature of between 40°° C. and 50° C.

In some embodiments according to the present invention the peptide when hydrolyzing the lactose in the milk-based substrate has a ratio of lactase to transgalactosylase activity of more than 1:1.

In some embodiments according to the present invention less than 80% of the lactose has been hydrolyzed when step c) is completed, and wherein more than 90% of the lactose has been hydrolyzed after one week.

### Numbered Embodiments

1. A peptide exhibiting beta-galactosidase enzyme activity, which has an amino acid sequence represented by SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, or enzymatically active fragments thereof, or an amino acid sequence of any one thereof having not more than 1, 2, 3, 4, 5, 6, 7, 8, 9, 10, 11, 12, 13, 14, 15, 16, 17, 18, 19, 20, 21, or 22 amino acid substitutions, additions or deletions.
2. A dimeric peptide exhibiting beta-galactosidase enzyme activity, which dimeric peptide consist of two peptides having an amino acid sequence represented by SEQ ID NO: 2 and 3, 5 and 6, 20 and 21, 23 and 24, 26 and 27, or 28 and 29; or enzymatically active fragments thereof, or an amino acid sequence of any one thereof having not more than 1, 2, 3, 4, 5, 6, 7, 8, 9, 10, 11, 12, 13, 14, 15, 16, 17, 18, 19, 20, 21, or 22 amino acid substitutions, additions or deletions.
3. The peptide or dimeric peptide according to embodiments 1 or 2, which has a beta-galactosidase activity measured as μM of glucose formed per second per μM of enzyme under conditions as given in example 8 described herein at a temperature of about 4° C. and a pH of 6.7, which activity is exceeding the activity of a beta-galactosidase enzyme defined by SEQ ID NO:34 OR SEQ ID NO:35 by at least about 20%, such as at least about 30%, such as at least about 40%, such as at least about 50%, such as at least about 60%, such as at least about 70%, such as at least about 80%, such as at least about 90%, such as at least about 100%, such as at least about 200%, such as at least about 300%, such as at least about 400%, such as at least about 500%.
4. The peptide or dimeric peptide according to any one of embodiments 1-3, which has a beta-galactosidase activity measured as μM of glucose formed per second per μM of enzyme under conditions as given in example 10 described herein at a temperature of about 4° C. and a pH of 5.5, which activity is exceeding the activity of a beta-galactosidase enzyme defined by SEQ ID NO:34 OR SEQ ID NO:35 by at least about 20%, such as at least about 30%, such as at least about 40%, such as at least about 50%, such as at least about 60%, such as at least about 70%, such as at least about 80%, such as at least about 90%, such as at least about 100%, such as at least about 200%, such as at least about 300%, such as at least about 400%, such as at least about 500%.
5. The peptide or dimeric peptide according to any one of embodiments 1-4, which has a beta-galactosidase activity measured as μM of glucose formed per second per μM of enzyme under conditions as given in example 13 described herein at a temperature of about 4° C. and a pH of 4.5, which activity is exceeding the activity of a beta-galactosidase enzyme defined by SEQ ID NO:34 OR SEQ ID NO:35 by at least about 20%, such as at least about 30%, such as at least about 40%, such as at least about 50%, such as at least about 60%, such as at least about 70%, such as at least about 80%, such as at least about 90%, such as at least about 100%, such as at least about 200%, such as at least about 300%, such as at least about 400%, such as at least about 500%.
6. The peptide or dimeric peptide according to any one of embodiments 1-5, which has a beta-galactosidase activity measured as μM of glucose formed per second per μM of enzyme under conditions as given in example 9 described herein at a temperature of about 43° C. and a pH of 6.7, which activity is exceeding the activity of a beta-galactosidase enzyme defined by SEQ ID NO:34 OR SEQ ID NO:35 by at least about 20%, such as at least about 30%, such as at least about 40%, such as at least about 50%, such as at least about 60%, such as at least about 70%, such as at least about 80%, such as at least about 90%, such as at least about 100%, such as at least about 200%, such as at least about 300%, such as at least about 400%, such as at least about 500%.
7. The peptide or dimeric peptide according to any one of embodiments 1-6, which has a beta-galactosidase activity measured as μM of glucose formed per second per μM of enzyme under conditions as given in example 12 described herein at a temperature of about 43° C. and a pH of 5.5, which activity is exceeding the activity of a beta-galactosidase enzyme defined by SEQ ID NO:34 OR SEQ ID NO:35 by at least about 20%, such as at least about 30%, such as at least about 40%, such as at least about 50%, such as at least about 60%, such as at least about 70%, such as at least about 80%, such as at least about 90%, such as at least about 100%, such as at least about 200%, such as at least about 300%, such as at least about 400%, such as at least about 500%.
8. The peptide or dimeric peptide according to any one of embodiments 1-7, which has a beta-galactosidase activity measured as μM of glucose formed per second per μM of enzyme under conditions as given in example 15 described herein at a temperature of about 43° C. and a pH of 4.5, which activity is exceeding the activity of a beta-galactosidase enzyme defined by SEQ ID NO:34 OR SEQ ID NO:35 by at least about 20%, such as at least about 30%, such as at least about 40%, such as at least about 50%, such as at least about 60%, such as at least about 70%, such as at least about 80%, such as at least about 90%, such as at least about 100%, such as at least about 200%, such as at least about 300%, such as at least about 400%, such as at least about 500%.
9. The peptide or dimeric peptide according to any one of embodiments 1-8, which has a beta-galactosidase activity measured as μM of glucose formed per second per μM of enzyme under conditions as given in example 6 described herein at a temperature of about 37° C. and a pH of 6.7, which activity is exceeding the activity of a beta-galactosidase enzyme defined by SEQ ID NO:34 OR SEQ ID NO:35 by at least about 20%, such as at least about 30%, such as at least about 40%, such as at least about 50%, such as at least about 60%, such as at least about 70%, such as at least about 80%, such as at least about 90%, such as at least about 100%, such as at least about 200%, such as at least about 300%, such as at least about 400%, such as at least about 500%.
10. The peptide or dimeric peptide according to any one of embodiments 1-9, which has a beta-galactosidase activity measured as μM of glucose formed per second per μM of enzyme under conditions as given in example 11 described herein at a temperature of about 37° C. and a pH of 5.5, which activity is exceeding the activity of a beta-galactosidase enzyme defined by SEQ ID NO:34 OR SEQ ID NO:35 by at least about 20%, such as at least about 30%, such as at least about 40%, such as at least about 50%, such as at least about 60%, such as at least about 70%, such as at least about 80%, such as at least about 90%, such as at least about 100%, such as at least about 200%, such as at least about 300%, such as at least about 400%, such as at least about 500%.
11. The peptide or dimeric peptide according to any one of embodiments 1-10, which has a beta-galactosidase activity measured as μM of glucose formed per second per μM of enzyme under conditions as given in example 14 described herein at a temperature of about 37° C. and a pH of 4.5, which activity is exceeding the activity of a beta-galactosidase enzyme defined by SEQ ID NO:34 OR SEQ ID NO:35 by at least about 20%, such as at least about 30%, such as at least about 40%, such as at least about 50%, such as at least about 60%, such as at least about 70%, such as at least about 80%, such as at least about 90%, such as at least about 100%, such as at least about 200%, such as at least about 300%, such as at least about 400%, such as at least about 500%.
12. The peptide or dimeric peptide according to any one of embodiments 1-11, derived from a bacteria of the genus Bifidobacterium, such as Bifidobacterium adolescentis, Bifidobacterium bifidum, Bifidobacterium breve, Bifidobacterium catenulatum, Bifidobacterium longum or from the genus Lactobacillus, such as L. sakei, L. amylovorus, L. delbrueckii subsp. bulgaricus, L. delbrueckii subsp. lactis, L. delbrueckii subsp. Indicus, L. crispatus, L. reuteri, L. helveticus or from Streptococcus thermophilus.
13. The peptide or dimeric peptide according to any one of embodiments 1-12, wherein said peptide or dimeric peptide exhibit a galactose inhibition less than 60%, such as less than 55%, such as less than 50%, such as less than about 45%, such as less than about 40%.
14. A nucleotide sequence which encodes a peptide or dimeric peptide as defined in any one of embodiments 1-13.
15. A host cell comprising a nucleotide sequence as defined in embodiment 14.
16. A method for producing a peptide or dimeric peptide as defined in any one of the embodiments 1-13, which method comprises the expression of a vector containing a nucleotide sequence as defined in embodiment 14 in a suitable host cell; and purifying said peptide or dimeric peptide from the expression products of said host cell.
17. A method for reducing the lactose content in a composition containing lactose, such as in a dairy products, comprising the step of contacting said composition with a peptide or dimeric peptide exhibiting beta-galactosidase enzyme activity, which peptide has an amino acid sequence represented by SEQ ID NO:1-33, or which dimeric peptide consist of two peptides having an amino acid sequence represented by SEQ ID NO: 2 and 3, 5 and 6, 20 and 21, 23 and 24, 26 and 27, or 28 and 29; or enzymatically active fragments thereof, or any sequence with at least 80% sequence identity to any one of said sequences or enzymatically active fragments; or a host cell expressing any one of said peptide or dimeric peptide, at a pH ranging from 3-10 and at a temperature ranging from 0° C.-140° C.
18. The method according to embodiment 17, wherein said composition is a dairy product selected from the group consisting of lactose-free milk, low-lactose milk, yoghurt, cheese, fermented milk products, dietary supplement and probiotic dietary products.
19. The method according to any one of embodiments 17-18, wherein said host cell is any one selected from a bacteria of the genus Bifidobacterium, such as Bifidobacterium adolescentis, Bifidobacterium bifidum, Bifidobacterium breve, Bifidobacterium catenulatum, Bifidobacterium longum or from the genus Lactobacillus, such as L. sakei, L. amylovorus, L. delbrueckii subsp. bulgaricus, L. delbrueckii subsp. lactis, L. delbrueckii subsp. Indicus, L. crispatus, L. reuteri, L. helveticus or from Streptococcus thermophilus.
20. The method according to any one of embodiments 17-19, wherein the lactose concentration is reduced to less than about 1%, such as to less than about 0.1% or lower, such as to less than about 0.01%.
21. Use of a peptide or dimeric peptide exhibiting beta-galactosidase enzyme activity, which peptide has an amino acid sequence represented by SEQ ID NO:1-33, or which dimeric peptide consist of two peptides having an amino acid sequence represented by SEQ ID NO: 2 and 3, 5 and 6, 20 and 21, 23 and 24, 26 and 27, or 28 and 29; or a sequence with at least 80% sequence identity to any one of said sequences; or a host cell expressing any one of said peptide or dimeric peptide for producing a dairy product with a reduced lactose content.
22. The use according to embodiment 21, wherein said dairy product is selected from the group consisting of lactose-free milk, low-lactose milk, yoghurt, cheese, fermented milk products, dietary supplement and probiotic dietary products.
23. The use according to any one of embodiments 21-22, wherein said host cell is any one selected from a bacteria of the genus Bifidobacterium, such as Bifidobacterium adolescentis, Bifidobacterium bifidum, Bifidobacterium breve, Bifidobacterium catenulatum, Bifidobacterium longum or from the genus Lactobacillus, such as L. sakei, L. amylovorus, L. delbrueckii subsp. bulgaricus, L. delbrueckii subsp. lactis, L. delbrueckii subsp. Indicus, L. crispatus, L. reuteri, L. helveticus or from Streptococcus thermophilus.
24. A method for producing a dairy product the method comprising the steps of
a) providing a milk-based substrate comprising lactose;
b) adding an peptide or dimeric peptide exhibiting beta-galactosidase activity, which peptide has an amino acid sequence represented by SEQ ID NO:1-33; or which dimeric peptide consist of two peptides having an amino acid sequence represented by SEQ ID NO: 2 and 3, 5 and 6, 20 and 21, 23 and 24, 26 and 27, or 28 and 29; or a sequence with at least 80% sequence identity to any one of said sequences to said milk-based substrate comprising lactose; and
c) treating said milk-based substrate with said peptide or dimeric peptide exhibiting beta-galactosidase activity.
25. The method according to embodiment 24, wherein said peptide or dimeric peptide is derived from any one bacteria of the genus Bifidobacterium, such as Bifidobacterium adolescentis, Bifidobacterium bifidum, Bifidobacterium breve, Bifidobacterium catenulatum, Bifidobacterium longum or from the genus Lactobacillus, such as L. sakei, L. amylovorus, L. delbrueckii subsp. bulgaricus, L. delbrueckii subsp. lactis, L. delbrueckii subsp. Indicus, L. crispatus, L. reuteri, L. helveticus or from Streptococcus thermophilus.
26. The method according to any one of embodiments 24-25, wherein step c) takes place at a pH within a range of 3-10, such as within a range of 3-9, such as within a range of 3-8, such as within a range of 3-7, such as within a range of 3-6, such as within a range of 3-5, such as within a range of 3-4, such as within a range of 4-10, such as within a range of 4-9, such as within a range of 4-8, such as within a range of 4-7, such as within a range of 4-6, such as within a range of 4-5, such as within a range of 5-10, such as within a range of 5-9, such as within a range of 5-8, such as within a range of 5-7, such as within a range of 5-6, such as within a range of 6-10, such as within a range of 6-9, such as within a range of 6-8, such as within a range of 6-7.
27. The method according to any one of embodiments 24-26, wherein step c) or a part of step c) takes place at a temperature of not more than about 25° C., such as not more than about 20° C., such as not more than about 18° C., such as not more than about 16° C., such as not more than about 14° C., such as not more than about 12° C., such as not more than about 10° C., such as not more than about 8° C., such as not more than about 7° C., such as not more than about 6° C., such as not more than about 5° C., such as not more than about 4° C., such as not more than about 3° C., such as not more than about 2° C.
28. The method according to any one of embodiments 24-27, wherein step c) or a part of step c) takes place at a temperature of at least about 25° C., such as at least about 30° C., such as at least about 35° C., such as at least about 40° C., such as at least about 45° C., such as at least about 50° C., such as at least about 55° C., such as at least about 60° C., such as at least about 65° C., such as at least about 70° C., such as at least about 75° C., such as at least about 80° C., such as at least about 85° C., such as at least about 90° C., such as at least about 95° C., such as at least about 100° C., such as at least about 110° C., such as at least about 120° C., such as at least about 130° C., such as at least about 120° C., such as at least about 130° C., such as at least about 135° C., such as at least about 140° C.
29. The method according to any one of embodiments 24-28, wherein said dairy product is selected from the group consisting of lactose-free milk, low-lactose milk, yoghurt, cheese, fermented milk products, dietary supplement and probiotic dietary products.
30. The method according to any one of embodiments 24-29, wherein said milk-based substrate is selected from fresh milk or raw milk obtained directly from a step of pasteurization, milk obtained directly after a step of ultra-heat treatment (UHT), or milk obtained directly after a step of fermentation.
31. The method according to any one of embodiments 24-30, wherein the galactose inhibition of said peptide or dimeric peptide is less than 60%, such as less than 55%, such as less than 50%, such as less than about 45%, such as less than about 40%.
32. The method according to any one of embodiments 24-31, wherein said dairy product is fermented milk product and said step b) is performed during or prior to fermentation.
33. The method according to embodiment 32, which method does not require the addition of further enzyme after fermentation.
34. The method according to any one of embodiments 24-31, wherein said dairy product is fermented milk product and said step b) is performed immediately following fermentation.
35. The method according to any one of embodiments 24-31, wherein said dairy product is fresh milk and said step b) is performed prior to, in conjunction with, or immediately following a step of pasteurization.
36. The method according to any one of embodiments 24-31, wherein said dairy product is ultra-heat treatment (UHT) milk and said step b) is performed prior to, in conjunction with, or immediately following a step of ultra-heat treatment.
37. The method according to any one of embodiments 24-36, wherein step c) is started at a temperature of between 40° C. and 100° C., such as at a temperature of between 50° C. and 100° C. such as at a temperature of between 60° C. and 100° C., such as at a temperature of between 70° C. and 100° C., such as at a temperature of between 80° C. and 100° C., such as at a temperature of between 40° C. and 90° C., such as at a temperature of between 40° C. and 80° C., such as at a temperature of between 40° C. and 70° C., such as at a temperature of between 40° C. and 60° C., such as at a temperature of between 40° C. and 50° C.
38. The method according to any one of embodiments 24-37, wherein the peptide or dimeric peptide when hydrolysing the lactose in the milk-based substrate has a ratio of lactase to transgalactosylase activity of more than 1:1.
39. The method according to any one of embodiments 24-38, wherein less than 80% of the lactose has been hydrolyzed when step c) is completed, and wherein more than 90% of the lactose has been hydrolyzed after one week.
40. A dairy product prepared by a method as defined in any one of embodiments 24-39.
41. A food product, such as a dairy product comprising a peptide or dimeric peptide exhibiting beta-galactosidase enzyme activity, which peptide has an amino acid sequence represented by SEQ ID NO:1-33, or which dimeric peptide consist of two peptides having an amino acid sequence represented by SEQ ID NO: 2 and 3, 5 and 6, 20 and 21, 23 and 24, 26 and 27, or 28 and 29; or a sequence with at least 80% sequence identity to any one of said sequences.
42. A food product, such as a dairy product comprising a host cell expressing a peptide or dimeric peptide exhibiting beta-galactosidase enzyme activity, which peptide has an amino acid sequence represented by SEQ ID NO:1-33, or which dimeric peptide consist of two peptides having an amino acid sequence represented by SEQ ID NO: 2 and 3, 5 and 6, 20 and 21, 23 and 24, 26 and 27, or 28 and 29; or a sequence with at least 80% sequence identity to any one of said sequences.
43. The food product according to embodiment 42, which is selected from beverages, infant foods, cereals, bread, biscuits, confectionary, cakes, food supplements, dietary supplements, probiotic comestible products, prebiotic comestible products, animal feeds, poultry feeds and medicaments, or a dairy product selected from the group consisting of lactose-free milk, low-lactose milk, dried milk powder, baby milks, yoghurt, ice cream, cheese, fermented milk products, dietary supplement and probiotic dietary products.

### Sequences

TABLE 1
The gene numbers with corresponding sequence identification number.

| Gene | Sequence | |
|---|---|---|
| number | Identity number | Species name |
| G4 | SEQ ID No 1 | Bifidobacterium adolescentis |
| G16 | SEQ ID No 2 (domain a) | Lactobacillus sakei |
| | SEQ ID No 3 (domain b) | |
| G35 | SEQ ID No 4 | Bifidobacterium adolescentis |
| G40 | SEQ ID No 5 (domain a) | Lactobacillus amylovorus |
| | SEQ ID No 6 (domain b) | |
| G44 | SEQ ID No 7 | Bifidobacterium bifidum |
| G51 | SEQ ID No 8 | Bifidobacterium bifidum |
| G57 | SEQ ID No 9 | Bifidobacterium breve |
| G62 | SEQ ID No 10 | Bifidobacterium catenulatum |
| G66 | SEQ ID No 11 | Bifidobacterium catenulatum |
| G83 | SEQ ID No 12 | Lactobacillus delbrueckii |
| | | subsp. bulgaricus |
| G84 | SEQ ID No 13 | Lactobacillus delbrueckii |
| | | subsp. lactis |
| G95 | SEQ ID No 14 | Lactobacillus delbrueckii |
| | | subsp. bulgaricus |
| G100 | SEQ ID No 15 | Lactobacillus delbrueckii |
| | | subsp. bulgaricus |
| G104 | SEQ ID No 16 | Lactobacillus delbrueckii |
| | | subsp. lactis |
| G108 | SEQ ID No 17 | Lactobacillus delbrueckii |
| | | subsp. bulgaricus |
| G109 | SEQ ID No 18 | Lactobacillus delbrueckii |
| | | subsp. bulgaricus |
| G118 | SEQ ID No 19 | Lactobacillus delbrueckii |
| | | subsp. lactis |
| G145 | SEQ ID No 20 (domain a) | Lactobacillus helvaticus |
| | SEQ ID No 21 (domain b) | |
| G158 | SEQ ID No 22 | Bifidobacterium lonpum |
| G224 | SEQ ID No 23 (domain a) | Lactobacillus reuteri |
| | SEQ ID No 24 (domain b) | |
| G256 | SEQ ID No 25 | Lactobacillus delbrueckii |
| | | subsp. lactis |
| G282 | SEQ ID No 26 (domain a) | Lactobacillus helvaticus |
| | SEQ ID No 27 (domain b) | |
| G334 | SEQ ID No 28 (domain a) | Lactobacillus crispatus |
| | SEQ ID No 29 (domain b) | |
| G335 | SEQ ID No 30 | Streptococcus thermophilus |
| G336 | SEQ ID No 31 | Lactobacillus delbrueckii |
| | | subsp. indicus |
| G11 | SEQ ID No 32 | Bifidobacterium adolescentis |
| G33 | SEQ ID No 33 | Bifidobacterium adolescentis |
| G600 | SEQ ID No 34 | Bifidobacterium bifidum |
| G500 | SEQ ID No 35 | Kluyveromyces lactis |

SEQ ID No. 1
MADTAELAIVHATTASASWLTDPTVFAANRKPAHSSHRYVIGETSEPKQSLDGEWKVRIEQARNVDVESAPFAAVDFEDGDFG
AIEVPGHLQMAGYLKNKYVNIQYPWDGHEDPQAPNIPENNHVATYRRRFALDQALARTLENDGTVSLTFHGAATAIYVWLDGT
FVGYGEDGFTPSEFDVTEALRNGNGNAADSPEAEHTLTVACYEYSSAWSLEDQDFWRLHGLFRTVELAAQPHTHVETVQLEAD
YTAADTAGTADTAELNAALTLRNSADAMTIESTLRDGDGNVVSESTQACNGEIALNSGKMTNIAPWSAESPTLYTLTVRVVGH
DGAITETVTQKIGFRTFRIENGIMTLNGKRIVFKGADRHEFDAKRGRAITREDMLSDVVFCKRHNINAIRTSHYPNQEYWYDL
CDEYGLYLIDETNMETHGTWVANNVERPEDGTPGSRPEWEGACVDRINSMMRRDYNHPSVLIWSLGNESSAGEVFRAMYRHAH
TIDPNRPVHYEGSVHMREFEDVTDIESRMYAHADEIERYLNDGSPAHTDGPKKPYISCEYMHAMGNSCGNMDEYTALERYPMY
QGGFTWDFIDQAIETKLPDGTTRMCYGGDFGDRPSDYEFSGDGLLFADRTPSPKAQEVKQLYANVKIVVSVDEARITNDNLFV
STGDYRFVLRILADGKPVWSTTRRFDVAAGESASFEVDWPVDDYRSNAEELVLEVSQQLGNACDWAPAGYELAFGQCVVAGAK
TTADAVDAAGAPADGTVTLGRWNAGVRGQGREALFSRTQGGMVSYTFGEREFVLRRPSITTFRPLTDNDRGAGHAFERAAWAV
AGKYARCVDCAIANRGENAVEATYTYELAIPQRTKVTVRYVADTAGLVSLDVEYPGEKNGDLPTIPAFGIEWALPVEYANLRF
YGAGPEETYADRRHAKLGVWSTTAGDDCAPYLLPQETGNHEDVRWAEITDDSGHGVRVKRGAGAKPFAMSLLPYSSTMLEEAL
HQDELPKPRHMFLRLLAAQMGVGGDDSWMSPVHEQYQLPADQPLSLNVQLKLF
(G16 Domain a)
SEQ ID No. 2
MQPNIQWLDTPAVFRVGQLPAHSDHRYYATLAEMAQQQSSFEQSLNGTWQFHYSVNAASRPKSFYELAFDAQDFEPITVPQHI
ELAGYEQLHYINTMYPWEGHYYRRPAFSTSDDKQHLGMFSEADYNPVGSYLHHFDLTPALRNQRVIIRFEGVEQAMYVWLNGQ
FIGYAEDSFTPSEFDLTPYLKETDNCLAVEVHKRSSAAFIEDQDFFRFFGIFRDVKLLAKPRTHLEDLWVIPEYDVVQQTGQV
KLRLQFSGDENRVHLRIRDQHQIILTADLTSAAQVNGLYKMPELVQAWSNQTPNLYTLELEVVDQAGETIEISQQPFGFRKIE
IKDKVMLLNGKRLVINGVNRHEWHPETGRTITAEDEAWDIACMQRNHINAVRTSHYPDRLSFYNGCDQAGIYMMAETNLESHG
SWQKMGAVEPSWNVPGSYDEWEAATLDRARTNFETFKNHVSILFWSLGNESYAGSVLEKMNAYYKQQDPTRLVHYEGVFRAPE
YKATISDVESRMYATPAEIKAYLDNAPQKPFILCEYMHDMGNSLGGMQSYIDLLSQYDMYQGGFIWDFIDQALLVTDPVTGQR
ELRYGGDFDDRPSDYEFSGDGLVFATRDEKPAMQEVRYYYGEHK
(G16 Domain b)
SEQ ID No. 3
MKNQQVRRLDTIMANTNKRLAVIFGDVTLGLKGPDFHYLFSYQTGGPESIRIQGKEWLYRSPKPTFWRATTDNDRGNQFPLKS
GMWLAADQFIACQSITVAIDGQTIPLPIAPENNRYCGQETAQEVTVTYTYQTITTPQTTVEVSYTIQASGKIRVAVTYHGQAG
LPSLPVFGLRFVMPTPATRFIYQGLSGETYPDRMAGGMAGEYEVTGLPVTPYLVPQDCGVHMATDWVTIYRQAVLDNRLREPV
ETGLKFKMVDQPFAFSCLPYTAEELENATHHSELPAPHRTVLSLLGAVRGVGGIDSWGSDVEAYYQIDATQDHHLEFETSF
SEQ ID No. 4
MADTAELAIVHATTASASWLTDPTVFAANRKPAHSSHRYVIGETSEPKQSLDGEWKVRIEQARNVDVESAPFAAVDFEDGDFG
AIEVPGHLQMAGYLKNKYVNIQYPWDGHEDPQAPNIPENNHVAIYRRRFALDAQLARTLENDGTVSLTFHGAATAIYVWLDGT
FVGYGEDGFTPSEFDVTEALRNGNGNAADSPEAEHTLTVACYEYSSASWLEDQDFWRLHGLFRTVELAAQPHTHVETVQLEAD
YTAADTAGTADTAELNAALTLRNPADAMTIESTLRDGDGNVVWESTQACNGEIALNSGKMTNIAPWSAESPTLYTLTVRVVGH
DGAITETVTQKIGFRTFRIENGIMTLNGKRIVFKGADRHEFDAKRGRAITREDMLSDVVFCKRHNINARITSHYPNQEYWYDL
CDEYGLYLIDETNMETHGTWVANNVERPGEDIPGSRPEWEGACVDRINSMMRRDYNHPSVLIWSLGNESSAGEVFRMAYRHAH
TIDPNRPVHYEGSVHMREFEDVTDIESRMYAHADEIERYLNDGSPAHTDGPKKPYISCEYMHAMGNSCGNMDEYTALERYPMY
QGGFIWDFIDQAIETKLPDGTTRMCYGGDFGDRPSDYEFSGDGLLFADRTPSPKAQEVKQLYANVKIAVSVDEARITNDNLFV
STGDYRFVLRILADGKPVWSTTRRFDVAAGESASFEVDWPVDDYRSNAEELVLEVSQLLGNACDWAPAGYELAFGQCVVAGAK
TTADAVDAAGAPADGTVTLGRWNAGVRGQGREALFSRTQGGMVSYTFGEREFVLRRPSITTFRPLTDNDRGAGHAFERAAWAV
AGKYARCVDCAIANRGENAVEATYTYELAIPQRTKVTVRYVADTAGLVSLDVEYPGEKNGDLPTIPAFGIEWALPVEYANLRF
YGAGPEETYADRRHAKLGVWSTTAGDDCAPYLLPQETGNHEDVRWAEITDDSGHGVRVKRGAGAKPFAMSLLPYSSTMLEEAL
HQDELPKPRHMFLRLLAAQMGVGGDDSWMSPVHEQYALPADQPLSLNVQLKLF
(G40 Domain a)
SEQ ID No. 5
MKANIKWLDDPEVFRINQLPAHSDHPFYKDYREWQNHSSSFKQSLNGAWQFHFSKDPQSRPIDFYKRSFDSSSFDTIPVPSEI
ELNGYAQNQYTNILYPWESKIYRKPAYTLGRGIKDGDFSQGKDNTVGSYLKHFDLNPALAGHDIHIQFEGVERAMYVYLNGHF
IGYAEDSFTPSEFDLTPYIQAKDNILAVEVFKHSTASWLEDQDMFRFSGIFRSVELLALPRTHLMDLDIKPTVVNDYHDGVFN
AKLHFMGKTSGNVHVLIEDIDGKTLLNKKLPLKSTVEIENETFANVHLWDNHDPYLYQLIIEVHDQDGKLVELIPYQFGFRKI
EITKDHVVLLNGKRLIINGVNRHEWDAKRGRSITLADMKQDIATFKHNNINAVRTCHYPNQIPWYYLCDQNGIYMMAENNLES
HGTWQKLGQVEATSNVPGSIPEWREVVVDRARSNYETFKNHTAILFWSLGNESYAGSNIAAMNKLYKDHDSSRLTHYEGVFHA
PEFKKEISDLESCMYLPPKEAEEYLQNPKKPLVECEYMHDMGTPDGGMGSYIKLIDKYPQYMGGFIWDFIDQALLVHDPVSGQ
DVLRYGGDFDDRHSDYEFSGDGLMFADRTPKPAMQEVRYYYGLHK
(G40 Domain b)
SEQ ID No. 6
MAYTNNLHVVYGEASLGVNGQDFAYLFSYERGGLESLKIKDKEWLYRTPTPTFWRATTDNDRGSGFNQKAAQWLGADMFTKCV
GIHVQVDDHRFDELPVAPINNQFSNQEFAHEVKVAFDYETLTTPATKVKIIYNINDFGHMTITMHYFGKKGLPPLPVIGMRFI
MPTKAKSFDYTGLSGETYPDRMAGAERGTFHIDGLPVTKYLVPQENGMHMQTNELVITRNSTQNNADKDGDFSLKITQTKQPF
NFSLLPYTAEELENATHIEELPLARRSVLVIAGAVRGVGGIDSWGSDVEEQYHIDPQEDHEFSFTLN
SEQ ID No. 7
MNTTDDQRKNGDPIVSPSIPTTAWLADPRVYAVHRLDAHSDHACWSRSPVDGESTDLRQSLDGEWRVRVETAPTGRFPDGTSD
GPDWESDVSPLFAAPGFDDSSFSRVQVPSHLETAGLLAPQYVNVQYPWDGHEDPKAPAIPEEGHVAVYRREFDADGEVAQAVR
EGRPVTLTFQGAATAIYVWLNGSFIGYAEDSFTPSEFDVEDAIKVDGNVLAVACYEYSSASWLEDQDFWRLHGLERSVELNAR
PAAHVADLHADADWDLATSRGSLSLDVLIDGAANAATADFALRDKNGTIVWRTATKADGTLEAEAEIDDAAPWSAERPDLYEL
SVTLLDADGAVLETARTRIGFREVAIEDGILKENGKRLVFRGVNRHEFDCRRGRAITEEDMLWDIRFMKRHNINAVRTSHYPN
QSRWYELCDEYGIYLIDEINLETHGSWNSPGDIPVGTSVPGDDEAWLGACIDRLDSMILRDRNHPSVEVWSLGNESYAGEVLK
AMSAHAHQLDPGRPVHYEGVNWNHAYDGISDFESRMYAKPAEIQDWLEHGDERGEASKPFVSCEYMHAMGNSCGGESEFIDLE
RYERYSGGFIWDYIDQGLVQRLPDGSERESVGGEWGDRPEDYEFVGNGIVFADRTPSPKAQEVKQLYSPVKLAPDGHGVTIEN
RNLFAGTDGYVFAARLLEDGHEIWHADYRFDVAAGDTQHHDIAFPDIDADGDTREVIYEVDLLLAEATAWAPAGYELAFGQLT
GTLNPEQDITETSHDDDGRATRILSRWNAGIRRDDEEILLSRTQGGIVSWKRDDREMVIRRPELVTFRPLTDNDRGNHSGFDR
AAWFAAGRYAIVTETKIHESDDGLVAEYQYELADPNHTPVSVTYHVNSDMRMQLTVEYPGNATDMASEPAFGIEWELPGEYDR
LRYYGPGPEETYRDRKQGGKLGIWDATAKASMAPYLMVQETGSHEDVRWLEATDIQGEGLRVTQRGDRHFTASLLPWNEYTIE
AARRHEDLPKPRHNYLRLLAAQMGVGGDDSWGAPVHTAYQLPAGRPLTLDVNLELI
SEQ ID No. 8
MNTTDDQRKNGDPIVSPSIPTTAWLADPRVYAVHRLDAHSDHACWSRSPVDGESTDLRQSLDGEWRVRVETAPTGRFPDGTSD
GPDWESDVSPLFAAPGFDDSSFSRVQVPSHLETAGLLAPQYVNVQYPWDGHEDPKAPAIPEEGHVAVYRREFDADGEVAQAVR
EGRPVTLTFQGAATAIYVWLNGSFIGYAEDSFTPSEFDVSDAIKVDGNVLAVACYEYSSASWLEDQDFWRLHGLFRSVELNAR
PAAHVADLHADADWDLATSRGSLSLDVLIDGAANAATADFALWDKNGTIVWHIVTKADGTLEAEAEIDDAAPWSAERPDLYEL
SVTLLDADGAVLETARTRIGFREVAIEDGILKENGKRLVFRGVNRHEFDCRRGRAITEEDMLWDIRFMKRHNINAVRTSHYPN
QSRWYELCDEYGIYLIDEINLETHGSWNSPGDIPVGTSVPGDDEAWLGACIDRLDSMILRDRNHPSVEVWSLGNESYAGEVLK
AMSAHAHRLDPGRPVHYEGVNWNHAYDGISDFESRMYAKPAEIQDWLEHGDERGEASKPFVSCEYMHAMGNSCGGESEFIDLE
RYERYSGGFIWDYIDQGLVQRLPDGSERESVGGEWGDRPEDYEFVGNGIVFADRTPSPKAQEVKQLYSPVKLAPDGHGVTIEN
RNLFAGTDGYVFAARLLEDGHEIWHADYRFDVAAGDTQHHDIAFPDIDADGDTREVIYEVDLLLAEATAWAPAGYELAFGQLT
GTLNPEQDITETSHDDDGRATRTLSRWNAGIRRDDKEILLSRTQGGIVSWKRDDREMVIRRPELVTFRPLTDNDRGNHSGFDR
AAWFAAGRYAIVTEIKIHESDDGLVAEYQYELADPNHTPVSVTYHVNSDMRMQLTVEYPGNATDMASEPAFGIEWELPGEYDR
LRYYGPGPEETYRDRKQGGKLGIWDATAKASMAPYLMVQETGSHELVRWLEATDIQGEGLRVTQRGDRHFTASLLPWNEYMIE
AARRHEDLPEPRHNYLRLLAAQMGVGGDDSWGAPVHTAYQLPAGRPLTLDVNLELI
SEQ ID No. 9
MTNSMQGKAXTIMINLQSAQQFSQAWLTDPRVFAVNRLAAHSSHKFYDHSPQCGEAMDLKQSLDGQWRVQMLDLADLADNELA
EAAFAQPGYDAAGFSPIEVPSALETKGFLNEQYVNQQYPWSGHESPVAPDVPKHNHVALYREEFSLEPKAAAVLEANKEAADD
AAKRRVTLTFQGAATAIVVWLNGAFIGYAEDSFTPSEFDVTDVLRDGVNTLAVACFEFSSASWLEDQDFWRLHGIFRSVELEA
QPLVHVNDLRVLADYDHTIGEGSLDVVALLRNAGTAAAVAATVLDAAGNTVWHSKLTAGADAETLTVKANVGKVNPWSAEEPT
LYTLQVVATDAAGQVIEAALQRIGFRHFAIEDGLMKLNGKRIVFKCVDRHEFDARTGRTIAEADMIEDIESFKRLNINAVRTS
HYPNETRWYELCDEYGIYVLDETNLETHGSWEDPGDVFQPARAEPGSKDEWRAACVDRTASMVRRDYNHPSVVIWSLGNEAFG
GDVFYSMRDTVHENDPFRPVHYEGTFNDPEFSAATDIMSRMYAKPDEIVKLYLGEDGKKPYISCEYSHSMGNSTGGLHLYTEL
ERYPLYQGGFIWDYVDQALWQDCGNGTERLAYGGDFEDRPNDYEFSGDGVMFADRTPSPKAQEVKQLYANVKLVPDESGVTIT
NDNLFISTASSLFTARVLVDGAERWHANYRFDVPAGETVREPIAFPKVTDLVALSGSAEVTYEVDQREAEATDWAPAGYELTF
GQYVAAVSFDDGAADAVVAGDAEVAADGFNAGIHTDFGEVLLSKTQGGMVSFKRDGREMVIRRPNLTTFRALTDNDRGNGSGF
ERAQWMAAGRYARVIGTSVEETADGKGLKATYSYELADAKHTPVTVHYEVDAALRVHLTVEYPGEADAATLPAFGEEWELPKQ
YDRLRFYGLGPEETYADRLHGAKLGVFSRIAAEDCAPYLLPQEEGNHEQVRWAEITDEYGHGMRVTAAGGTRFATSLLPYSSL
MFEDALHQNELPKPRHTFLRLLAAQMGVGGDDTWGAPVHDEFQVPADQPLKLDVTLELI
SEQ ID No. 10
MTQRRSYRWPQPLAGQQARIWYGGDYNPDQWPEEVWDDDVRLMKKAGVNLVSVGIFSWAKIETSEGVYDFDWLDRIIDKLGEA
GIAVDLASATASPPMWLTQAHPEVLWKDYRGDVCQPGARQHWRPTSPVTREYALKLCRAMAEHYKGNPYVVAWHVSNEYGCHN
RFDYSEDAERAFRKWCEERYGTIDAVNDAWGEAFWAQRMNDFTEIVPPRFIGDGNFMNPGKLLDFKRFSSDALKAFYVAERDA
LAEICPDLPLTTNFMVSAAGSVLDYDDWGREVDFVSNDHYFIPGEAHLDELAFSASLVDGIARKDPWFLMEHSTSAVNWRPVN
YRKEPGQLVRDSLAHVAMGADAVCYFQWRQSKAGAEKFHSANVPHIGEDSAVFRDVCELGADLNTLADNGLLGTKLAKSKVAV
VFDYESEWATEHTATPTQKVHHVDEPLQWFRALADHGVTADVVPVSSNWDEYEVVVLPSVYILSEETTRRVRDYVVNGGRLIV
TYYTGLSDEXDHVWLGGYPGSIRDVVGVRVEEFMPMGDDFPGVPDCLGLSNGAVAHDIADVIGSVDGTATVLETFRDDPWTGM
DGAPAIVANTFGEGRSVYVGARLGRDGIAKSLPEIFESLGMAEEGENDSRVLRVEREGSDGSRFVFSFNRTHEAVQIPFEGKI
VVSSFAEVSGENVSIKPNGVIVTKQ
SEQ ID No. 11
MANSNRVEHASETWLTDATVFEVNRTPAHSNHKCFTHDPQSGEHSDLTQSLDGEWRVEIVQASDIDFNEEPFVAENFDDSSFC
RACVPGHLQMAGLLKNKYVNIQYPWDGHENPLEPNVPENNHVALYRRKFVVSKRLADTKESEGSVSIVFEGMATAIYVWVNGL
FAGYGEDGFTPNEFDITDLLHDGENVVAVACYEYSSASWLEDQDFWRLHGLFRSVELTAQPEVHVENMQLEADWDAESGTASL
DAALSVRNASDAATISATLKDSEGNVVWEASENADANTTFASGSLQGLEPWSAESPSLYELEVNVIDQAGNIVEAAVQKVGFR
RFRIENGIMTLNGKRIVFKGADRHEFDAKRGRSITEQDMIDDVIFCKRHNINAIRTSHYPNQERWYDLCDEYGIYLIDETNLE
THGSWCLPGDVVTAETAVPGSKAHWEGACVDRVNSMVRRDYNHPSVVIWSLGNESYTGDVFRAMYKHVHDIDPNRPVHYEGVT
KNRDYDDVTDIETRMYEHADVVEEYLKNDPQKPYISCEYMHAMGNSVGNLDEYIALERYPHYQGGFIWDFIDQAIYATQPDGS
TRLCYGGDFGDRPSDYEFSGNGLVFADRTPTPKAQEVKQLYSNVHIDVTDRSVSIKNDNLFISTGGYQFVLRILADGEPVWQS
ERRFDVPADSACTFDVEWPVDLYRANADELVLEVSQRLAEATDWAPAGYELAFGQTIVAGTKAAEDAALPADGIVTVGRWNAG
VQGSGREILLSRTQGGLVSYTFDGHEFVLRRPAITTFRALTDNDRGAGHGFERAQWMVAGRYARCVDNVIEQVDEDTLKAVYT
YELAEPQCTXVTVGYTADTTGRLNLHVEYPGESGELPTIPAFGEEWTLPVQYSNLRFFGAGPEETYQDRKHAKLGVWSEDAFK
DHAPYLMPQETGNHEEVRWAEITDENGHGLRVSRANGAAPFAVSLQPYSSFMIEEAQEQDELPAPKHMFLRVLAAQMGVGGDD
SWMSPVHSQYHITADQPISLDVNLELI
SEQ ID No. 12
MSNKLVKEKRVDQADLANLTDPEVYEVNTIPPHSDHESFQSQEELEEGASSLVQSLDGDWLIDYAENGQGPVNFYAEDFDDSN
FKSVKVPGNLELQGFGQPQYVNVQYPWDGSEEIFPPQIPSKNPLASYVRYFDLDEAFWDKEVSLKFDGAATAIYVWLNGHFVG
YGEDSFTPSZFMVTKFLKKENNRLAVALYKYSSASWLEDQDFWRMSGLFRSVTLQAKPRLHLEDLKLTASLTDNYQKGKLEVE
ANIAYRLPNASFKLEVRDSEGDLVAEKLGPIRSEQLEFTLADLPVAAWSAEKPNLYQVRLYLYQAGSELEVSRQEVGFRNFEL
KDGIMYLNGQRIVFKGANRHEFDSKLGRAITEEDMIWDIKTMKRSNINAVRCSHYPNQSLFYRLCDKYGLYVIDEANLESHGT
WEKVGGHEDPSFNVPGDDQHWLGASLSRVKNMMARDKNHASILEWSLGNESYAGTVFAQMADYVRKADPTRVQHYEGVEHNRK
FDDACQIESRMYAPAKVIEEYLINKPAKPFISVEYAHAMGNSVGDLAAYTALEKYPHYQGGFIWDWIDQGLEKDGHLLYGGDF
DDRPEDYEFCGNGLVFADRTESPKLANVKALYANLKLEVKDGQLFLKNDNLFTNSSSYYFLTSLLVDGKLTYQSRPLTFGLEP
GESGEFALPWPEVADEKGEVVYRVTAHLKEDLPWADEGFEVAEAEEVAQKLPEFKPEGRPDLVDSDYNLGLKGNNFQILFSKV
KGWPVSLKYAGREYLKRLPEFTFWRALTDNDRGAGYGYDLARWENAGKYARLKDISCEVKEDSVLVKTAFTLPVALKGDLTVI
YEVDGRGKIAVTADFPGAEEAGLLPAFGENLALPKELTDYRYYGLGPNESYPDRLEGNYLGIYQGAVKKNFSPYLRPQETGNR
SKVRWYQLFDEKGGLEFTANGADLNLSALPYSAAQIEAADHAFDLINNYTWVRALSAQMGVGGDDSWGQKVHPEFCLDAQKAR
QLRLVIQPLLLK
SEQ ID No. 13
MSNKLVKEKRVDQADLANLTDPEVYEVNTIPPHSDHESFQSQEELEEGKSSLVQSLDGNWLIDYAENGQGPINFYAEDFDDSN
FKSVKVPGNLELQGFGQPQYVNIQYPWDGSEEIFPPQVPSKNPLASYVRYFDLDEALWDKEVSLKFAGAATAIYVWLNGHFVG
YGEDSFTPSEFMVTKFLKKEGNRLAVALYKYSSASWLEDQDFWRLSGLTRSVTLEAKPLLHLEDLKLTASLTDNYQKGKLEVE
ANIAYRLPNASFKLEVRDSEGDLVAEKVGPIRSEKLGFSLADLPVAAWSAEKPNLYQVRLYLYQAGSELEVSRQEVGFRNFEL
KDGIMYLNGQRIVFKGVNRHEFDSKLGRAITEADMIWDIKTMKQSNINAVRCSHYPNQSLFYRLCDKYGLYVIDEANLESHGT
WEKVGHEDPSFNVPGDDQHWLGASLSRVKNMMARDKNHASILIWSLGNESYAGTVFAQMADYVRKADPIRVQHYEGVTHNRKE
DDATQIESRMYAPAKEIEEYLIKKPAKPFISVEYAHAMGNSVGDLAAYTALEKYPHYQGGFIWDWIDQGLEKDGHLLYGGDFD
DRPTDYEFCGDGLVFADRITSPKLANVKALYSNLKLEVKDGQLFIKNDNLFTNSSAYYFLASLLVDGKLIYQSQPLTFGLEPG
ESGTFVLPWPEVEDEKGEIVYQVTAHLKEDLPWADEGFTVAEAEEAVTXLPEFYPAGRPELVDSDFNEGLKGNGFRILFSKAK
GWPVSIKYAGREYLKRLPEFTFWRALTDNDRGAGYGYDLAKWENAGKYARLQDISYEIKENSALVKTTFTLPVALKGDLTITY
EVDSLGKIAVTANFPGAVENGLLPAFGLNFALPKELSDYRYYGLGPNESYADRLEGSYLGIYQGAVEKNFIPYLRPQEAGNRS
KVRYYQLFDEEGGLEFTANGADLNLSALPYSAAQIEAADHAFELTNNYTWVRALAAQMGVGGDDSWGQKVHPEFCEDAQEARQ
LKLVEQPLLLK
SEQ ID No. 14
MSNKLVKEKRVDQAELAWLTDPEVYEVNTIPPHFDHESFQSQEELEEGKSSLVQSLDGDWLIDYAENGQGPVNFYAEDFDDSN
FKSVKVPGNLELQGFGQPQYVNVQYPWDGSEEIFPPQIPSKNPLASYVRYFDLDEAFWDKEVSLKFDGAAIAIYVWLNGHFVG
YGEDSFTPSEFMVTKFLKKENNRLAVALYKYSSASWLEDQDFWRMSGLFRSVTLQAKPRLHLEDLKLTASLTDNYQKGKLEVE
ANIAYRLPNASFKLEVRDSEGDLVAEKLGPIRSEQLEFILADLPVAAWSAEKPNLYQVRLYLYQAGSLLEVSRQEVGFRNFEL
KDGIMYLNGQRIVFKGANRHEFDSKLGRAITEEDMIWDIKTMKRSNINAVRCSHYPNQSLFYRLCDKYGLYVIDEANLESHGT
WEKVGGHEDPSFNVPGDDQHWLGASLSRVKNMMARDKNHASILEWSLGNESYAGTVFAQMADYVRKADPTRVQHYEGVEHNRK
FDDATQIESRMYAPAKVIEEYLINKPAKPFISVEYAHAMGNSVGDLAAYTALEKYPHYQGGFIWDWIDQGLEKDGHLLYGGDF
DDRPTDYEFCGNGLVFADRIESPKLANVKALYANLKLEVKDGQLFLKNDNLFTNSSSYYFLISLLVDGKLIYQSRPLIFGLEP
GESGTFALPWPEVADEKGFVVYRVTAHLKEDLPWADEGFTVAEAEEVAQKLPEFKPEGRPDLVDSDYNLGLKGNNFQILFSKV
KGWPVSLKYAGREYLKRLPEFIFWRALIDNDRGAGYGYDLARWENAGKYARLKDISCEVKEDSVLVKTAFILPVALKGDLTVT
YEVDGRGKIAVTADFPGAEEAGLLPAFGENLALPKELTDYRYYGLGPNESYPDRLEGNYLGIYQGAVKKNFSPYLRPQETGNR
SKVRWYQLFDEKGGLEFIANGADLNLSALPYSAAQIEAADHAFELINNYTWVRALSAQMGVGGDDSWGQKVHPEFCLDAQKAR
QLRLVIQPLLLK
SEQ ID No. 15
MSNKLVKEKRVDQAELAWLTDPEVYEVNTIPPHSDHESFQSQEELEEGKSSLVQSLDGDWLIDYAENGQGPVNFYAEDFDDSN
FKSVKVPGNLELQGFGQPQYVNVQYPWDGSEEIFPPQIPSKNPLASYVRYFDLDEAFWDKEVSLKFDGAAIAIYVWLNGHFVG
YGEDSFTPSEFMVIKFLKKENNRLAVALYKYSSASWLEDQDFWRMSGLFRSVILQAKPRLHLEDLKLIASLIDNYQKGKLEVE
ANIAYRLPNASFKLEVRDSEGDLVAEKLGPIRSEQLEFILADLPVAAWSAEKPNLYQVRLYLYQAGSLLEVSRQEVGFRNFEL
KDGIMYLNGQRIVFKGANRHEFDSKLGRAIIEEDMIWDIKTMKRSNINAVRCSHYPNQSLFYRLCDKYGLYVIDEANLESHGT
WEKVGGHEDPSFNVPGDDQHWLGASLSRVKNMMARDKNHASILIWSLGNESYAGIVFAQMADYVRKADPIRVQHYEGVTHNRK
FDDATQIESRMYAPAKVIEEYLINKPAKPFISVEYAHAMGNSVGDLAAYTALEKYPHYQGGFIWDWIDQGLEKDGHLLYGGDF
DDRPTDYEFCGNGLVFADRIESPKLANVKALYANLKLEVKDGQLFLKNDNLFTNSSSYYFLISLLVDGKLIYQSRPLIFGLEP
GESGTFALPWPEVADEKGEVVYRVTAHLKEDLPWADEGFTVAEAEEVAQKLPEFKPEGRPDLVDSDYNLGLKGNNFQILFSKV
KGWPVSLKYAGREYLKRLPEFIFWRALIDNDRGAGYGYDLARWENAGKYARLKDISCEVKEDSVLVKTAFILPVALKGDLTVT
YEVDGRGKIAVTADFPGAEEAGLLPAFGENLALPKELTDYRYYGLGPNESYPDRLEGNYLGIYQGAVKKNFSPYLRPQETGNR
SKVRWYQLFDEKGGLEFTANGADLNLSALPYSAAQIEAADHAFELINNYTWVRALSAQMGVGGDDSWGQKVHPEFCLDAQKAR
QLRLVIQPLLLK
SEQ ID No. 16
MSNKLVKEKRVDQADLAWLIDPEVYEVNTIPPHSDHESFQSQEELEEGKSSLVQSLDGDWLIDYAENGQGPVNFYAEDFDDSN
FKSVKVPGNLELQGFGQPQYVNIQYPWDGSEEIFPPQVPSKNPLASYVRYFDLDEAFWDKEVSLKFAGAATAIYVWLNGHFVG
YGEDSFTPSEFMVTKFLKKENNRLAVALYKYSSASWLEDQDFWRLSGLFRSVTLQAKPLLHLEDLKLTASLTDNYQKGKLEVE
ANIAYRLPNASFKLEVRDSEGDLVAEKLGPIRSEQLEFILADLPVAAWSAEKPNLYQVRLYLYQAGSLLEVSRQEVGFRNFEL
KDGIMYLNGQRIVFKGVNRHEFDSKLGRAITEEDMIWDIKTMKRSNINAVRCSHYPNQSLFYRLCDKYGLYVIDEANLESHGT
WEKVGHEDPSFNVPCDDQHWLGASLSRVKNMMARDKNHASILIWSLGNESYAGIVFAQMADYVRKADPIRVQHYEGVTHNRKE
DDATQIESRMYAPAKEIEEYLIKKPAKPFISVEYAHAMGNSVGDLAAYTALEKYPHYQGGFIWDWIDQGLEKDGHELYGGGDF
DDRPCDYEFCGNGLVFADRTTSPKLANVKALYSNLKLEVKDGQLFLKNDNLFTNSSAYYFLTSLLVDGKLTYQSQPLTFGLEP
GESGEFVLPWPEVEDEKGEIVYQVTAHLKEDLPWADEGFEVAEAEEAVTKLPEFYPAGRPELVDSDFNLGLKGNGFRILFSKA
KGWPVSIKYAGREYLKRLPEFTFWRALTDNDRGAGYGYDLAKWENAGKYARLQDISYEIKENSVLVKIAFTLPVALKGDLTII
YEVDSLGKIAVTANFPGAVENGLLPAFGLNFALPKELSDYRYYGLGPNESYADRLEGSYLGIYQGAVEKNFTPYLRPQEAGNR
SKVRYYQLFDEESGLEFTANGADLNLSALPYSAAQIEAADHAFELSNNYTWVRALAAQMGVGGDDSWGQKVHPEFCLDAQEAR
QLKLVIQPLLLK
SEQ ID No. 17
MSNKLVKEKRVDQADLAWLTDPEVYEVNTIPPHSDHESFQSQEELEEGKSSLVQSLDGDWLIDYAENGQGPVNFYAEDFDDSN
FKSVKVPGNLELQGFGQPQYVNVQYPWDGSEEIFPPQIPSKNPLASYVRYFDLDEAFWDKEVSLKFDGAATAIYVWLNGHFVG
YGEDSFTPSEFMVIKFLKKENNRLAVALYKYSSASWLEDQDFWRMSGLFRSVTLQAKPRLHLEDLKLTASLTDNYQKGKLEVE
ANIAYRLPNASFKLEVRDSEGDLVAEKLGPIRSEQLEFTLADLPVAAWSAEKPNLYQVRLYLYQAGSELEVSRQEVGFRNFEL
KDGIMYLNGQRIVFKGVNRHEFDSKLGRAITEEDMIWDIKTIKRSNINAVRCSHYPNQSLFYRLCDKYGLYVIDEANLESHGT
WEKVGGHEDPSFNVPGDDQHWLGASLSRVKNMMARDKNHASILEWSLGNESYAGTVFAQMADYVRKADPTRVQHYEGVCHNRK
FDDACQIESRMYAPAKVIEEYLINKPAKPFISVEYAHAMGNSVGDLAAYTALEKYPHYQGGFIWDWIDQGLEKDGHLLYGGDF
DDRPEDYEFCGNGLVFADRTESPKLANVKALYANLKLEVKDGQLFLKNDNLFTNSSSYYFLTSLLVDGKLTYQSRPLTFGLEP
GESGEFALPWPEVADEKGEVVYRVTAHLKEDLPWADEGFEVAEAEEVAQKLPEFKPEGRPDLVDSDYNLGLKGNNFQILFSKV
KGWPVSLKYAGREYLKRLPEFTFWRALTDNDRGAGYGYDLARWENAGKYARLKDISCEVKEDSVLVKTAFTLPVAEKGDLTVT
YEVDGRGKIAVTADFPGAEEAGLLPAFGENLALPKELIDYRYYGLGPNESYPDRLEGNYLGIYQGAVKKNFSPYLRPQETGNR
SKVRWYQLFDEKGGLEFTANGADLNLSALPYSAAQIEAADHAFDLINNYTWVRALSAQMGVGGDDSWGQKVHPEFCLDAQKAR
QLRLVIQPLLLK
SEQ ID No. 18
MSNKLVKEKRVDQADLANLTDPEVYEVNTIPPHSDHESFQSQEELEEGASSLVQSLDGDWLIDYAENGQGPVNFYAEDFDDSN
FKSVKVPGNLELQGFGQPQYVNVQYPWDGSEEIFPPQIPSKNPLASYVRYFDLDEAFWDKEVSLKFDGAATAIYVWLNGHFVG
YGEDSFTPSEFMVTKFLKKENNRLAVALYKYSSASWLEDQDFWRMSGLFRSVTLQAKPRLHLEDLKLTASLTDNYQKGKLEVE
ANIAYRLPNASFKLEVRDSEGDLVAEKLGPIGSEQLEFTLADLPVAAWSAEKPNLYQVRLYLYQAGSELEVSRQEVGFRNFEL
KDGIMYLNGQRIVFKGVNRHEFDSKLGRAITEEDMIWDIKTIKRSNINAVRCSHYPNQSLFYRLCDKYGLYVIDEANLESHGT
WEKVGGHEDPSFNVPGDDQHWLGASLSRVKNMMARDKNHASILEWSLGNESYAGTVFAQMADYVRKADPTRVQHYEGVEHNRK
FDDACQIESRMYAPAKVIEEYLINKPAKPFISVEYAHAMGNSVGDLAAYTALEKYPHYQGGFIWDWIDQGLEKDGHLLYGGDF
DDRPCDYEFCGNGLVFADRTESPKLANVKALYANLKLEVKDGQLFLKNDNLFTNSSSYYFLTSLLVDGKLTYQSRPLTFGLEP
GESGEFALPWPEVADEKGEVVYRVTAHLKEDLPWADEGFEVAEAEEVAQKLPEFKPEGRPDLVDSDYNLGLKGNNFQILFSKV
KGWPVSLKYAGREYLKRLPEFTFWRALTDNDRGAGYGYDLARWENAGKYARLKDISCEVKEDSVLVKTAFTLPVALKGDLTVI
YEVDGRGKIAVTADFPGAEEAGLLPAFGENLALPKELTDYRYYGLGPNESYPDRLEGNYLGIYQGAVKKNFSPYLRPQETGNR
SKVRWYQLFDEKGGLEFTANGADLNLSALPYSAAQIEAADHAFELINNYTWVRALSAQMGVGGDDSWGQKVHPEFCLDAQKAR
QLRLVIQPLLLK
SEQ ID No. 19
MSNKLVKEKRVDQADLAWLTDPEVYEVNTIPPHSDHESFQSQEELEEGKSSLVQSLDGNWLIDYAENGQGPINFYAEDFDDSN
FKSVKVPGNLELQGFGQPQYVNIQYPWDGSEEIFPPQVPSKNPLASYVRYFDLDEALWDKEVSLKFAGAATAIYVWLNGHFVG
YGEDSFTPSEFMVIKFLKKEGNRLAVALYKYSSASWLEDQDFWRLSGLFRSVILEAKPLLHLEDLKLTASLTDNYQKGKLEVE
ANIAYRLPNASFKLEVRDSEGDLVAEKVGPIRSEKLDFSLADLPVAAWSAEKPNLYQVRLYLYQAGSLLEVSRQEVGFRNFEL
KDGIMYLNGQRIVFKGVNRHEFDSKLGRAIIEADMIWDIKTMKQSNINAVRCSHYPNQSLFYRLCDKYGLYVIDEANLESHGT
WEKVGHEDPSFNVPGDDQHWLGASLSRVKNMMARDKNHASILIWSLGNESYAGIVFAQMADYVRKADPIRVQHYEGVTHNRKF
DDAIQIESRMYAPAKEIEEYLIKKPAKPFISVEYAHAMGNSVGDLAAYTALEKYPHYQGGFIWDWIDQGLEKDGHLLYGGDFD
DRPTDYEFCGDGLVFADRTTSPKLANVKALYSNLKLEVKDGQLFIKNDNLFTNSSAYYFLTSLLVDGKLTYQSQPLTFGLEPG
ESGIFALPWPEVEDEKGEIVYQVIAHLKEDLPWADEGFIVAEAEEAVTKLPEFYPAGRPELVDSDFNLGLKGNGFRILFSKAK
GWPVSIKYAGREYLKRLPEFTFWRALIDNDRGAGYGYDLAKWENAGKYARLQDISYEIKENSALVKIAFILPVALKGDLTIIY
EVDSLGKIAVTANFPGAVENGLLPAFGLNFALPKELSDYRYYGLGPNESYADRLEGSYLGIYQGMVEKNFIPYLRPQEAGNRS
KVRYYQLFDEEGGLEFTANGADLNLSALPYSAAQIEAADHAFELTNNYTWVRALAAQMGVGGDDSWGQKVHPEFCLDAQEARQ
LKLVEQPLLLK
(G145 Domain a)
SEQ ID No. 20
MQANTNWLDNPEVFRVNQLPAHSDHPFFRDYREWQKQHSSYQQSLNGKWKFHFSANPMDRPQDFYQRDFDSSNFDSIPVPSEi
ELSNYTQNQYINVLFPWEGKIFRRPAYALDPNDHEEGSFSKGADNIVGSYLKRFDLSSALIGKDVHIKFEGVEQAMYVWLNGH
FVGYAEDSFTPSEFDLTPYIQDKDNLLAVEVFKHSTASWLEDQDMFRFSGIFRSVELLGIPATHLMDMDLKPRVADNYQDGIF
NLKLHFIGKKAGSFHLLVKDIKGHTLLEKNEDIKENVQINNEKFENVHLWNNHDPYLYQLLIEVYDEQQNLLELIPFQFGFRR
IEISPEKVVLLNGKRLIINGVNRHEWDAKRGRSITMSDMTTDINTFKENNINAVRTCHYPNQIPWYYLCDQNGIYVMAENNLE
SHGTWQKMGEIEPSDNVPGSIPQWKEAVIDRARNNYETFKNHTSILFWSIGNESYAGDNIIAMNEFYKSHDDTRLVHYEGVVH
RPELKDKISDVESCMYLPPKKVEEYLQNDPPKPFMEGEYMHDMGNSDGGMGSYIKLLDKYPQYFGGFIWDFIDQALLVHDEIS
GHDVLRYGGDFDDRHSDYEFSGDGLMFADRIPKPAMQEVRYYYGLHK
(G145 Domain b)
SEQ ID No. 21
MDYTNNQLHIIYGDATFGVNGKDFQYIFSYERGGLESLKVHGKEWLYRVPTPTFWRAITDNDRGSGFNLKAAQWLGADMFTKC
TDIHLKVDRHDFAELPIAPFNNKFSNHEYAKSAEISFTYQTLTEPATNAKIIYNIDDVGHIKVTMRYYGKKGLPPLPVEGIRL
TMPTAATGFDYEGLSGETYPDRMAGAKEGKEHIDGLPVTEYLVPQENGMHMQTKKLTINRETTQNNVDRINEKFSESIQQAEK
PFNFSCLPYTAEELENATHIEELPLVRRTVLVIAGAVRGVGGIDSWGTDVESAYHINPELDEEFSFIEN
SEQ ID No. 22
MTDVSHVDRASQAWLTDPTVFEVNRTPAHSSHKWYARDPQSGQWSDLKQSLDGEWRVEVVQAADINLEEEPATAESFDDSSFE
RIQVPGHLQTAGLMNHKYVNVQYPWDGHENPLEPNIPENNHVALYRRKFTVSAPVANAKQAGGSVSIVFEGMATAIYVWVNGA
FVGYGEDGFTPNEFDITELLHDGENVVAVACYEYSSASWLEDQDFWRLHGLFRSVELAARPEVHIENTQIEADWDPEAGTASL
DAALEVLNAADAATVRATLKDADGNTVWQTTGDAEAQTAESSGPLQGIAPWSAESPTLYELDVDVIDQAGDVIECTSQKVGFR
RFRIEDGILTINGKRIVFKGADRHEFDAEQGRAITEQDMEDDVVFCKRHNINSIRTSEYPNQERWYEECDEYGIYEIDEANLE
AHGSWSLPGDVLTEDTIVPGSKREWEGACVDRVNSMMRRDYNHPSVLIWSLGNESYVGDVFRAMYKHVHDIDPNRPVHYEGVT
HNRDYDDVTDIETRMYSHADEIEKYLKDDPKKPYLSCEYMHAMGNSVGNMDEYTALERYPKYQGGFIWDFIDQAIYATQPDGT
RSLRYGGDFGDRPSDYEFSGDGLLFANRKPSPKAQEVKQLYSNVHIDVTKDSVSVKNDNLFTATGDYVFVLSVLADGKPVWQS
TRRFDVPAGETRTFLVANPVAAYRADARELVLQVSQRLAKATDWAESGYELAFGQTVVPADATATPDTKPADGTITVGRWNAG
VRGAGREVLLSRTQGGMVSYTFAGNEFVERRPAITTFRPLTDNDRGAGHGFERVQWLGAGRYARCVDNVLEQIDDSTLKGTYT
YELAEAQRTXVTVSYTAHIDGRVNLHVEYPGEQGDLPTIPAFGEEWTLPVQYTNLRFFGTGPAETYLDRKHAKLGVWSENAFA
DHAPYLMPQETGNHEDVRWAEITDDHGHGMRVSRADGAAPFAVSLLPYSSFMLEEAQEQDELPKPKHMFLRVLAAQKGVGGDD
SWMSPVHPQYHIPADKPISLDVDLELI
(G224 Domain a)
SEQ ID No. 23
MDADEKWLDEPETFRVNQLPAHSDHYYYGNYDEWRHNNSRFAQNLDGQWQFNFAENLRERENDFYKMDYDSSSFGTIEVPSEI
ELNNYAQNNYINTLIPWEGKIYRRPAYIESPDDAQEGSFSDGDDNTIGEYLKHFDLDPSLRGKQVRIRFDGVERAMYVWLNGH
FIGYAEDSFTPSEFDLTPYIQDEGNVLAVEVFKHSTASWEEDQDMFRFSGIFRSVNLLAQPLVHVEDENIRPIVTDNYQDGIF
NVELQLHGEXTGNVNVRVIDNDGNTLVNETEPVDSTVKVQDQFLENVHLWDNHDPYLYQLLIEIRDDEGNLVELVPYRFGFRR
TEINKDHVVLLNGQRLIINGVNRHEWDARRGRAITMDDMESDIHTFKENNINAVRTCEYPDQIPWYYECDDNGIYMMAENNLE
SHATWQKMGAIEPSYNVPGSVPQWRDVVVDRARTNYETFKNHPSILFWSLGNESYAGDNIVKMNEFYKKEDDSRLVHYEGVCH
TPEYRDRISDVESWMYLPPKEVEEYLKNNPDKPFMECEYMHDMGNSDGGMGSYISLLDKYPQYFGGFIWDFIDQALLVKDPVS
GQEVMRYGGDFDDRHSDYEFSGDGLMFADRTPKPAMQEVRYYYGLHK
(G224 Domain b)
SEQ ID No. 24
MAYTNKLRVIYGDAILGLSGDGFHYIFSYERGGLESLKLNGKEWLYREPMPTFWRATTDNDRGSGFNIRSAQWLAADTFHKCV
GIDLEVDNQHFAELPIAPITNEFSDPVSAESVKIKYTFAELTVPAIQVTVIYEVNGQGEIKVTMHYYGHEDLPGLPVVGMRFI
MPTVATGFDYQGLSGETYPDRMAGATEGTFEVDGLPVTKYLVPQENGMHMATHALTITRDSTQNNADHSREPFSLTVKQDAQP
FAFSCLPYTAEELENATHIEELPLARRTVLVVAGAVRGVGGIDSWGADVEEQYHIPADRDVEFSFVLNAK
SEQ ID No. 25
MSNKLVKEKRVDQADLANLTDPEVYEVNTIPPHSDHESFQSQEELEEGASSLVQSLDGNWLIDYAENGQGPINFYAEDFDDSN
FKSVKVPGNLELQGFGQPQYVNIQYPWDGSEEIFPPQVPSKIPLASYVRYFDLDEALWDKEVSLKFAGAATAIYVWLNGHFVG
YGEDSFTPSEFMVTKFLKKEGNRLAVALYKYSSASWLEDQDFWRLSGLFRSVTLEAKPLLHLEDLKLTASLTDNYQKGKLEVE
ANIAYRLPNASFKLEVRDSEGDLVAEKVGPIRSEKLDFSLADLPVAAWSAEKPNLYQVRLYLYQAGSELEVSRQEVGFRNFEL
KDGIMYLNGQRIVFKGVNRHEFDSKLGRAITEADMIWDIKTMKQSNINAVRCSHYPNQSLFYRLCDKYGLYVIDEANLESHGT
WEKVGHEDPSFNVPGDDQHWLGASLSRVKNMMARDKNHASILIWSLGNESYAGTVFAQMADYVRKADPIRVQHYEGVTHNRKF
DDATQIESRMYAPAKEIEEYLIKKPAKPFISVEYAHAMGNSVGDLAAYTALEKYPHYQGGFIWDWIDQGLEKDGHLLYGGDFD
DRPTDYEFCGDGLVFADRITSPKLANVKALYSNLKLEVKDGQLFIKNDNLFTNSSAYYFLTSLLVDGKLTYQSQPLTFGLEPG
ESGTFALPWPEVEDEKGEIVYQVTAHLKEDLPWADEGFTVAEAEEAVTXLPEFYPAGRPELVDSDFNEGLKGNGFRILFSKAK
GWPVSIKYAGREYLKRLPEFTFWRALTDNDRGAGYGYDLAKWENAGKYARLQDISYEIKENSALVKTTFTLPVALKGDLIITY
EVDSLGKIAVTANFPGAVENGLLPAFGLNFALPKELSDYRYYGLGPNESYADRLEGSYLGIYQGMVEKNFTPYLRPQEAGNRS
KVRYYQLFDEEGGLEFTANGADLNLSALPYSAAQIEAADHAFELTNNYTWVRALAAQMGVGGDDSWGQKVHPEFCEDAQEARQ
LKLVEQPLLLK
(G2S2 Domain a)
SEQ ID No. 26
MQANENWLDNPEVFRVNQLPAHSDHPFFRDYREWQKQHSSYQQSLNGKWKFHFSANPMDRPQDFYQRDFDSSNFDSIPVPSEI
ELSNYTQNQYINVLFPWEGKIFRRPAYAEDPNDHEEGSFSKGADNTVGSYLKRFDLSSALIGKDVHIKFEGVEQAMYVWLNGH
FVGYAEDSFTPSEFDLTPYIQEKDNLLAVEVFKHSTASWLEDQDMFRFSGIFRSVELLGIPATHLMDMDLKPRVADNYQDGIF
NLKLHFIGKKAGSFHLLVKDIKGHTLLEKNEDIKENVQINNEKFENVHLWNNHDPYLYQLLIEVYDEQQNLLELIPFQFGFRR
TEISPEKVVLLNGKRLIINGVNRHEWDAKRGRSITMSDMETDINTFKENNINAVRTCEYPNQIPWYYECDQNGIYVMAENNLE
SHGTWQKMGEIEPSDNVPGSIPQWKEAVIDRARNNYETFKNHTSILFWSLGNESYAGDNIIAMNEFYKSEDDTRLVHYEGVVH
RPELKDKISDVESCMYLPPKKVEEYLQNDPPKPFMECEYMHDMGNSNGGMDSYIKLLDKYPQYFGGFIWDFIDQALLVHDEIS
GHDVLRYGGDFDDRHSDYEFSGDGLMFADRKPKPAMQEVRYYYGLHK
(G2S2 Domain b)
SEQ ID No. 27
MDYTNNQLHIIYGDATFGVNGKDFQYIFSYERGGLESLKVHGKEWLYRVPTPTFWRATTDNDRGSGFNLKAAQWLGADMFTKC
TDIHLKVDRHDFAELPIAPFNNKFSNHEYAKSAEISFTYQTLTEPATNAKIIYNIDDGGHIKVTMRYYGKKGLPPEPVEGIRL
TMPTAATGFDYEGLSGETYPDRMAGAKEGKFHIDGLPVTEYLVPQENGMHMQTKKLIINRETTQNNVDRTNEKFSESIQQAEK
PFNFSCLPYTAEELENATHIEELPLVRRTVLVIAGAVRGVGGIDSWGTDVESAYHINPDLDEEFSFIEN
(G334 Domain a)
SEQ ID No. 28
MKANEKWLDDPEVFRINQLPAHSDHPFYKDYREWQKHSSSFKQSLNGAWQFHFSKDPQSRPIDFYKLSFDSSSFDTIPVPSEI
ELNGYAQNQYTNILYPWESKIYRKPAYTLGRGIKDGDFSQGKDNTVGSYLKHFDLNPALAGEDIHIQFEGVERAMYVYLNGHF
EGYAEDSETPSEFDLTPYIQAKDNILAVEVEKHSTASWLEDQDMFRESGIFRSVELLALPRIHLMDLDIKPTVVNDYHDGVEN
AKLHFMGKTSGNVHVLIEDIDGKTLLNKKLPLKSTVEIENETFANVHLWDNHDPYLYQLIIEVHDQDGKLVELIPYQFGFRKI
EITKDHVVLLNGKRLIINGVNREEWDAKRGRSITLADMKQDIACFKHNNINAVRTCHYPNQIPWYYLCDQNGIYMMAENNLES
HGTWQKLGQVEATSNVPGSIPEWREVVVDRARSNYETFKNHTAELFWSLGNESYAGSNIAAMNKLYKDHDSSRLTHYEGVFHA
PEFKKEISDLESCMYLPPKEAEEYLQNPKKPLVECEYMHDMGNSDGGIGSYIKLIDKYPQYMGGFIWDFIDQALLVHDPVSGQ
DVLRYGGDFDDRHSDYEFSGDGLMFADRTPKPAMQEVRYYYGLHK
(G334 Domain b)
SEQ ID No. 29
MAYTNNLHVVYGEASLGVNGQDFAYLFSYERGVLESLKIKDKEWLYRTPTPTFWRATTDNDRGSGFNQKAAQWLGADMFTKCV
GIHVQVDDHQFDELPIAPINNQFSNQEFAHEVKVAFDYEELTTPAIKVXIIYNINDAGHMTITMHYFGKKGLPPLPVIGMRFI
MPTKAKSFDYTGLSGETYPDRMAGAERGTFEEDGLPVTKYLVPQENGMHMQTNELVITRNSTQNNADKDGDFSLKITQEKQPF
NFSLLPYTAZELENATHIEELPLARRSVEVIAGAVRGVGGIDSWGSDVEEQYHIDPEQDHEFSFILN
SEQ ID No 30
MNMTKIQTYLNDPKIVSVNTVDAHSDHKYFESLEEFSEGEMKLRQSLNGKWKIHYAQNTNQVLKDFYKTEFDETDLNFINVPG
MNMTKIQTYLNDPKIVSVNTVDAHSDHKYFESEEEFSEGEMKLRQSLNGKWKIHYAQNTNQVLKDFYKIEFDETDENFENVPG
HLELQGFGSPQYVNIQYPWDGKEFLRPPQVPQESNAVASYVKHFTLNDALKDKKVFISFQGVATSIFVWVNGNFVGYSEDSFT
PSEFEISDYLVEGDNKLAVAVYRYSIASWLEDQDFWRLYGIFRDVYLYAIPKVHVQDLFVKGDYDYQTKAGQLDIDLKEVGDY
EDKKIKYVLSDYEGIVTEGDASVNGDGELSVSLENLKIKPWSAESPKLYDLILHVLDDDQVVEVVPVKVGFRRFEIKDKLMLL
NGKREVFKGVNRHEFNARIGRCITEEDMEWDEKVMKQHNENAVRTSHYPNQTRWYELCDEYGLYVIDEANLETHGTWQKLGLC
EPSWNIPASEPEWLPACLDRANNMFQRDKNEASVIIWSCGNESYAGKDIADMADYFRSVDNIRPVHYEGVAWCREFDYITDIE
SRMYAKPADIEEYLITGKLVDLSSVSDKHFASGNLTNKPQKPYTSCEYMHTMGNSGGGLQLYTDLEKYPEYQGGFIWDFIDQA
TYKTLPNGSEFLSYGGDWHDRPSDYEFCGNGIVFADRTLTPKLQTVKHLYSNIKIAVDEKSVIIKNDNLFEDLSAYTFLARVY
EDGRKVSESEYHFDVKPGEEATFPVNFVVEASNSEQIYEVACVLREATKWAPKGHEIVRGQYVVEKISTETPVKAPINVVEGD
FNIGIQGQNFSILLSRAQNTLVSAKYNGVEFIEKGPKLSFTRAYTDNDRGAGYPFEMAGWKVAGNYSKVTDTQIQIEDDSVKV
TYVHELPGLSDVEVKVTYQVDYKGRIFVTANYDGKAGLPNFPEFGLEFAIGSQFINLSYYGYGAEESYRDKLPGAYLGRYEIS
VEKIFAPYLMPQESGNHYGIREFIVSDDNHNGEKFIALNKAFEFSALRNSIEQIENARHQYELQESDAIWIKVLAAQMGVGGD
DSWGAPVHDEFLLSSADSYQLSFMIEPLN
SEQ ID No 31
MNNKLAQVKRVDQADLAWLIDPEIYEVNTIPPHSDHESFQSLEELEEGKSSLVQSLDGDWLIDYAENGEGPANFYEENFDDSS
FKSVKVPGNLELQGFGQPQYVNVQYPWDGSDEIFPPMIPSKNPVASYVRYFDLEEAFWDKEVSLKFAGAAIAIYVWLNGHFVG
YGEDSFTPSEFMVTKFLKKEGNRLAVALYKYSSASWLEDQDFWRMSGLFRSVTLEAKPLLHLQDLKLTASLTNDYQKGSLQVE
ADIDYRLPNSSFKLELRDSAGELVAEKVGPIRSEKLDFSLADLPVAAWSAEEPNLYQVRL3LYQQGSELEVSRQEVGFRNFEL
KDGIMYLNGKRIVFKGVNRHEFDSKLGRAITEADMIWDIKIMKQSNINAVRCSHYPNQSIFYHLCDKYGLYVIDEANLESHGT
WEKVGGHEDPSFNVPGDDQRWLGASLSRVKNMMARDKNHASILEWSLGNESYAGKVFAQMADYVRQADPTRVQHYEGVEHNRK
FDDATQIESRMYAPAKEIEEYLIKKPAKPFVSCEYAHAMGNSVGDLAAYTALEKYPHYQGGFIWDWIDQGLEKEGHLLYGGDF
DDRPSDYEFCGDGLVFADRITSPKLANVKALYSNLKLELKDGQLFLKNDNLFTNSSAYYFLTSLLVDGKLIYQSQPLIFALEP
GESGEFALPWPEVEDEKGEIVYQVTAHLKEDLPWADEGFEVAEAEEAVTKLPEFYPAGRPELVDSDYNLGIKGNGFRILFSKA
KGWPVSIKYAGREYLKRLPEFTFWRALTDNDRGAGYGYDLAKWENAGKYARLQDISYEIKENSVLVKTAFTLPVALKGDLTIT
YEVDSLGKIAVTANFPGAVENGLLPAFGLNFALPKELSDYRYYGLGPNESYADRLEGSYLGIYQGAVEKNFTPYLRPQEVGNR
SKVRYYQLFDEEGGLEFTANGANLNLSAEPYSAAQIEAADHAFELINNYTWVRALAAQMGVGGDDSWGQKVHPEFCLDAQEAR
QLKLVIQPLFTE
SEQ ID NO: 32
MADTAELAIVHATTASASWLTDPTVFAANRKPAHSSHRYVIGETSEPKQSLDGEWKVRIEQARNVDVESAPFAAVDFEDGDFG
AIEVPGHLQMAGYLKNKYVNIQYPWDGHEDPQAPNIPENNHVAIYRRRFALDAQLARTLENDGTVSLTFHGAATAIYVWLDGT
FVGYGEDGFTPSEFDVTEALRNGNGNAADSPEAEHTLTVACYEYSSASWLEDQDFWRLHGLFRTVELAAQPHTHVETVQLEAD
YTAADTAGTADTAELNAALTLRNPADAMTIESTLRDGDGNVVWESTQACNGEIALNSGKMTNIAPWSAESPTLYTLTVRVVGH
DGAIIETVTQKIGFRTFRIENGIMTLNGKRIVFKGADRHEFDAKRGRAITREDMLSDVVFCKRHNINAIRTSHYPNQEYWYDL
CDEYGLYLIDETNMETHGTWVANNVERPEDGIPGSRPEWEDACVDRINSMMRRDYNHPSVLIWSLGNESSAGEVFRAMYRHAH
TIDPNRPVHYEGSVHMREFEDVTDIESRMYAHADEIERYLNDGSPAHTDGPKKPYISCEYMHAMGNSCGNMDEYTALERYPMY
QGGFIWDFIDQAIETKLPDGTTRMCYGGDFGDRPSDYEFSGDGLLFADRTPSPKAQEVKQLYANVKIAVSVDEARITNDNLFV
STGDYRFVLRILADGKPVWSTTRRFDVAAGESASFEVDWPVDDYRSNAEELVLEVSQQLGNACDWAPAGYELAFGQCVVAGAK
TTADAVDAAGAPADGTVTLGRWNAGVRGQGREALFSRTQGGMVSYTFGEREFVLRRPSITTFRPLTDNDRGAGHAFERAAWAV
AGKYARCVDCAIANRGENAVEATYTYELAIPQRTKVTVRYVADTAGLVSLDVEYPGEKNGDLPTIPAFGIEWALPVEYANLRF
YGAGPEETYADRRHAKLGVWSTTAGDDCAPYLLPQETGNHEDVRWAEITDDSGHGVRVKRGAGAKPFAMSLLPYSSTMLEEAL
HQDELPKPRHMFLRLLAAQMGVGGDDSWMSPVHEQYQLPADQPLSLNVQLKLF
SEQ ID NO: 33
MANETRIEHASETWLADSTVFEVNRVPAHSDHKCYAHDSQTNEWSDLRQSLDGEWRVEVVQASDIEFNEEPFVRENFDDSAFE
RIQVPGHLQMAGLMNNKYVNIQYPWDGHENPAEPNIPENNHVALYRKTFTMANRLADTKNAGGTVSIVFHGMATAIYVWVNGM
FVGYGEDGFTPNEFDITEMLHDGENVVAVACYEYSSASWLEDODFWRLHGLFRSVELAAQPHVHIENMQIESDWDPESGSASL
DAALTVRNAADAATISATLKDSDGNVVWETANCADPDTSISTGSLNGIRPWSAEDPVLYEFEVTVIDHAGNIAEVAVQKVGFR
RFRIEDGIMTINGKRIVFKGADRHEFDPKRGRAITEQDMIDDVVFCKRHNLNAIRTSHYPNQERWYELCDEYGIYLIDETNLE
THGSWCLPGDVLTEETAVPGSKAHWEGACVDRVNSMVRRDYNHPSVLIWSLGNESYTGDVFRAMYKRVHDIDPNRPVHYEGVT
HNRDYNDVTDIETRMYAHADAIEEYLKNDPQKPYISCEYMHAMGNSCGNMDEYTALERYPKYQGGFIWDFIDQAIYATQPDGT
TSLRYGGDFGDRPSDYEFSGNGLVFADRKPTPKAQEVKQLYSNVHIDVAEDSVTIKNDNLFTSTGEYTFVLRVLADGEPVWQS
ERRFDVPAGSTEKLDVDWPLDLYRDGASELVLEVSQRLAKATNWAVAGYELAFGQTVVAGSKKASAPVKPVDGIVTVGRWNVG
VQGSGREVLLSRTQGGLVSYTFNNREFVLRRPAVTTFRALTDNDRGAGHGFERAQWLGAGRYARCIGNEIEQIDENTVKASYT
YELATPQRTKVTVSYTADTTGRVNLHVEYPGEPGDLPTIPAFGIEWTLPVQYSNLRFFGAGPEETYQDRKHAKLGVWSTDAFK
DHAPYLMPQETGNHEDVRWAEITDEKGHGLRISRAEGAEPFAMSLQPYSSFMLEEAQHQDELPAPKHMFLRVLAEQMGVGGDD
SWMSPVHPQYHIPADQPISLDVDLDLI
(Reference enzyme)
SEQ ID NO: 34
MVEDATRSDSTTQMSSTPEVVYSSAVDSKQNRTSDFDANWKFMLSDSVQAQDPAFDDSAWQQVDLPHDYSITQKYSQSNEAES
AYLPGGTGWYRKSFTIDRDLAGKRIAINFDGVYMNATVWFNGVKLGTHPYGYSPFSFDLTGNAKFGGENTIVVKVENRLPSSR
WYSGSGIYRDVTLTVTDGVHVGNNGVAIKTPSLATQNGGNVTMNLTTKVANDTEAAANITLKQTVFPKGGKTDAAIGTVTTAS
KSIAAGASADVTSTITAASPKLWSIKNPNLYTVRTEVLNGDTVLDTYDTEYGFRWTGFDATSGFSLNGEKVKLKGVSMHHDQG
SLGAVANRRAIERQVEILQKMGVNSIRTTHNPAAKALIDVCNEKGVLVVEEVFDMWNRSKNGNTEDYGKWFGQTIAGDNAVLG
GDKDETWAKFDLTSTINRDRNAPSVIMWSLGNEMMEGISGSVSDFPATSAKLVAWTKAADSTRPMTYGDNKIKANWNESNTMG
DNLTANGGVVGTNYSDGANYDKIRTTHPSWAIYGSETASAINSRGIYNRTTGGAQSSDKQLTSYDNSAVGWGAVASSAWYDVV
QRDFVAGTYVWTGFDYLGEPTPWNGTGSGAVGSWPSPKNSYFGIVDTAGFPKDTYYFYQSQWNDDVHTLHILPAWNENVVAKG
SGNKVPVVVYTDAAKVKLYFTPKGSTEKRLIGEKSFTKKTTAAGYTYQVYEGTDKDSTAHKNMYLTWNVPWAEGTISAEAYDE
NNRLIPEGSTEGNASVTTTGKAAKLKADADRKTITADGKDLSYIEVDVTDANGHIVPDAANRVTFDVKGAGKLVGVDNGSSPD
HDSYQADNRKAFSGKVLAIVQSTKEAGEITVTAKADGLQSSTVKIATTAVPGTSTEKTVRSFYYSRNYYVKTGNKPILPSDVE
VRYSDGTSDRQNVTWDAVSDDQIAKAGSFSVAGTVAGQKISVRVTMIDEIGALLNYSASTPVGTPAVLPGSRPAVLPDGTVTS
ANFAVHWTKPADTVYNTAGTVKVPGTATVFGKEFKVTATIRVQRSQVTIGSSVSGNALRLTQNIPADKQSDTLDAIKDGSTTV
DANTGGGANPSAWTNWAYSKAGHNTAEITFEYATEQQLGQIVMYFFRDSNAVRFPDAGKTKIQISADGKNWTDLAATETIAAQ
ESSDRVKPYTYDFAPVGATFVKVTVTNADTTTPSGVVCAGLTEIELKTATSKFVTNTSAALSSLTVNGTKVSDSVLAAGSYNT
PAIIADVKAEGEGNASVTVLPAHDNVIRVITESEDHVTRKTFTINLGTEQEFPADSDERD
(Reference enzyme)
SEQ ID No: 35
MSCLIPENLRNPKKVHENRLPTRAYYYDQDIFESLNGPWAFALFDAPLDAPDAKNLDWETAKKWSTISVPSHWELQEDWKYGK
PIYTNVQYPIPIDIPNPPTVNPTGVYARTFELDSKSIESFEHRLRFEGVDNCYELYVNGQYVGFNKGSRNGAEFDIQKYVSEG
ENLVVVKVFKWSDSTYIEDQDQWWLSGIYRDVSLLKLPKKAHIEDVRVTTTFVDSQYQDAELSVKVDVQGSSYDHINFTLYEP
EDGSKVYDASSLLNEENGNTTFSTKEFISFSTKKNEETAFKINVKAPEHWTAENPTLYKYQLDLIGSDGSVIQSIKHHVGFRQ
VELKDGNITVNGKDILFRGVNRHDHHPRFGRAVPLDFVVRDLILMKKFNINAVRNSHYPNHPKVYDLFDKLGFWVIDEADLET
HGVQEPFNRHTNLEAEYPDTKNKLYDVNAHYLSDNPEYEVAYLDRASQLVLRDVNHPSIIIWSLGNEACYGRNHKAMYKLIKQ
LDPTRLVHYEGDLNALSADIFSFMYPTFEIMERWRKNHTDENGKFEKPLILCEYGHAMGNGPGSLKEYQELFYKEKFYQGGFI
WEWANHGIEFEDVSTADGKLHKAYAYGGDFKEEVHDGVFIMDGLCNSEHNPTPGLVEYKKVIEPVHIKIAHGSVTITNKHDFI
TTDHLLFIDKDTGKTIDVPSLKPEESVTIPSDTTYVVAVLKDDAGVLKAGHEIAWGQAELPLKVPDFVTETAEKAAKINDGKR
YVSVESSGLHFILDKLLGKIESLKVKGKEISSKFEGSSITFWRPPTNNDEPRDFKNWKKYNIDLMKQNIHGVSVEKGSNGSLA
VVTVNSRISPVVFYYGFETVQKYTIFANKINLNTSMKLTGEYQPPDFPRVGYEFWLGDSYESFEWLGRGPGESYPDKKESQRF
GLYDSKDVEEFVYDYPQENGNHTDTHFLNIKFEGAGKLSIFQKEKPFNFKISDEYGVDEAAHACDVKRYGRHYLRLDHAIHGV
GSEACGPAVLDQYRLKAQDFNFEFDLAFE

### EXAMPLES

### General Material and Methods

### Molecular Cloning and Genetic Techniques

Techniques for restriction enzyme digestions, ligation, transformation and other standard molecular biology manipulations were based on methods described in the literature (Maniatis et al. “Molecular cloning: a laboratory manual, 2nd edition” Cold Spring Harbor Laboratory Press, Cold Spring Harbor, NY, 1989; Sambrook and Russell “Molecular Cloning: A Laboratory Manual, 3rd edition” Cold Spring Harbor Laboratory Press, Cold Spring Harbor, NY 2001; Miller “Experiment in molecular genetics” Cold Spring Harbor Laboratory Press, 1972); or as suggested by the manufacturer. The PCR was carried out in a DNA thermal cycler obtained from (Bio-Rad, USA). DNA sequencing was performed by LGC, Berlin, Germany. Proteins were analyzed by polyacrylamide gel electrophoresis (PAGE) under the denaturation conditions using sodium dodecyl sulphate on gels containing 10% SDS (Mini-PROTEAN® TGX stain-free™ gel, Biorad, USA). Protein concentrations were determined using BCA method by following the protocol supplied with the kit.

### Bacterial Strains, Plasmid and Growth Conditions

Escherichia coli strain TOP10 (Invitrogen) was used for the cloning and isolation of plasmids. The beta-galactosidase deficient E. coli strain BW25113 (Δ(araD-araB)567, ΔlacZ4787(::rrnB-3), λ-, rph-1, λ(rhaD-rhaB)568, hsdR514) (Datsenko KA, Wanner BL; 2000, Proc Natl Acad Sci U.S.A. 97:6640-6645) was used in combination with the pBAD/His vector (obtained from Invitrogen™ Life Technologies Corporation Europe BV) for recombinant protein production.

### Growth Media for Protein Expression

2×PY medium containing (16 g/L BD BBL™ Phyton™ Peptone, 10 g/L Yeast Extract, 5 g/L NaCl) was used for the recombinant protein production. The growth medium was supplemented with ampicillin (100 μg/ml) to maintain the plasmid. Protein production was initiated by adding 0.05% of arabinose in to the culture medium.

### Example 1: Construction of the Expression Vector for the Production of Lactases

The genomic DNA of the lactic acid bacteria or bifidobacteria was extracted using commercial genomic extraction kit by following the supplied protocol (DNeasy, Qaigen, Germany). The lactase gene was amplified by PCR using two synthetic primers, using the purified genomic DNA source as biomass, and the PCR reagents were supplied in the Phusion U Hot start DNA polymerase (Thermo Scientific, USA) kit. The lactase gene was cloned into the start codon of the expression vector pBAD/His using the USER cloning method (Nour-Eldin HH, Geu-Flores F, Halkier BA, Plant Secondary Metabolism Engineering, Methods in Molecular Biology, 643; 2010), resulting in the expression construct. With the USER cloning method long, complementary overhangs in both PCR product and destination vector were generated. These overhangs can anneal to each other to form a stable hybridization product which was used to transform into E. coli without ligation. For the generation of overhangs in the PCR product, a single deoxyuradine residue is included in the upstream region of each primer to amplify target DNA. The lactase gene was amplified using the forward primer (5′-ATTAACCAUGCGACGCAACTTCGAATGGCC-3′, SEQ ID NO:36) and reverse primer (ATCTTCTCUTTACCGCCTTACCACGAGCACG, SEQ ID NO:37) containing a uridine at 9th position (as shown in bold), followed by with the lactase gene sequence. In parallel, the vector DNA was PCR amplified using the forward (5′-AGAGAAGAUTTTCAGCCTGATACAGATTAAATC-3′, SEQ ID NO:38) and reverse primer (5′-ATGGTTAAUTCCTCCTGTTAGCCCAAAAAACGG-3′, SEQ ID NO:39) pair containing single deoxyuracil residue at 9th positions (as highlighted in bold) followed by vector DNA sequence. The PCR products were purified using the commercial PCR purification kit (Qiagen, Denmark). The purified PCR products (lactase gene and the vector DNA) were mixed in equimolar amount and incubated with a commercial USER enzyme mix (New England Biolabs, USA) by following the supplied protocol. These enzymes remove the uracil residue and also the short fragment upstream of the uridine, thereby creating complementary overhang in the PCR products. These complementary overhangs anneal with each other resulting in the pBAD-lactase expression vector. Aliquots of the ligation mixture were transformed into chemically competent E. coli TOP 10 cells. Transformants were selected at 37° C. on LB-Amp plates (LB; Luria-Bertani, Amp; 100 μg/ml ampicillin). The following day, colony PCR was carried out using a small biomass from the overnight grown transformant using the vector primers (primer 1: 5′-CGGCGTCACACTTTGCTATGCC-3′, SEQ ID NO:40 and primer 2: 5′-CCGCGCTACTGCCGCCAGGC-3′, SEQ ID NO:41). The positive clones from the colony PCR were cultured in 5 mL LB-Amp medium and plasmid DNA was isolated from the cells. The cloned lactase gene was sequenced to verify that no additional mutations had been introduced during the amplification of the gene. The plasmid DNA was transformed in to the expression host E. coli strain BW25113.

### Example 2: Expression of Lactases in E. coli Expression Host

The lactase enzyme was produced in E. coli BW25113 using the pBAD expression system. Freshly transformed E. coli BW25113 cells carrying the plasmid DNA were collected from a Lb-Amp plate using a sterile loop and used to inoculate 5 mL of Lb-Amp medium. The overnight grown culture (200 μL) was used to inoculate 50 mL 2×PY medium (containing 100 μg/mL ampicillin) in a 250 mL flask in a shaker (Innova® 42). The culture was grown at 37° C. at 220 rpm until the OD600 reached between 0.6-0.8. The lactase expression was initiated by adding 0.05% arabinose into the culture medium and the cells were cultured for additional 16-20 hours at 18° C. at 180 rpm. Cells were harvested by centrifugation (5000 rpm, 10 min at 4° C.) and were stored at −20° C. until further use.

### Example 3: Protein Purification Using Immobilized Metal Affinity Chromatography

Cells from 50 mL culture was thawed on ice and the cells were lysed using 10 mL mixture of lysis buffer (BugBuster® (Novagen) containing 2 mg/mL Lysozyme (Sigma Aldrich), 1 unit Benzonase (Sigma Aldrich), and 1× Complete Protease inhibitor cocktail (EDTA-free, Roche)) by incubating the cells at room temperature for 30 min. After 30 min, the cell debris was removed by centrifugation at 16000 rpm for 20 min at 4° C. The obtained supernatant was filtered through 0.45 μm pore diameter filter. A gravity flow Ni-Sepharose (GE Healthcare) column was prepared with 1 mL slurry by washing out the ethanol and water. The column was then equilibrated with washing buffer (50 mM of NaH₂PO₄, pH 8.0 containing 300 mM of NaCl and 20 mM of Imidazole). The cell-free extract was applied to the column and the non-bound proteins were eluted from the column. The column was washed with 20 ml of washing buffer and the retained proteins were eluted with 3.5 mL of elution buffer (50 mM of NaH₂PO₄, pH 8.0 containing 300 mM of NaCl and 250 mM of imidazole). The collected fractions were analyzed by SDS-PAGE on gels containing 10% acrylamide and those contained the purified lactase enzymes combined together. The buffer was exchanged against the storage buffer (50 mM KH₂PO₄ buffer pH 7.0 containing 10 mM NaCl, 1 mM MgCl₂), using a prepacked PD-10 desalting G-25 gel filtration column (GE Healthcare). The purified enzymes were stored at 4° C. until further use.

### Example 4: Protein Purification Using Gel Filtration Chromatography

Cells from 50 mL culture was thawed on ice and the cells were lysed using 10 mL mixture of lysis buffer (BugBuster® (Novagen) containing 2 mg/ml lysozyme, 1 unit Benzonase (Sigma Aldrich), and 1× Complete Protease inhibitor cocktail (EDTA-free, Roche)) by incubating the cells at room temperature (25° C.) for 30 min. After 30 min, the cell debris was removed by centrifugation at 16000 rpm for 20 min at 4° C. The obtained supernatant was filtered through 0.45 μm pore diameter filter. The clear cell-free extract was concentrated by filtering through a 30000 Dalton filter (Vivaspin 20, GE Healthcare) by following the supplied protocol. A gravity flow Sephadex G50 superfine (Pharmacia Chemicals, Sweden) column was prepared with 1 g of column material (prepared by boiling in 100 ml water for 1 hour, cooled to room temperature). A column was prepared by applying 20 ml of the cooled slurry on a 30 mL filtration column. The column was washed with MilliQ water and equilibrated with wash buffer B (50 mM of NaH₂PO₄ buffer, pH 7.0). 500 μL of the concentrated supernatant was applied on the column and allowed the supernatant to enter in the column bed. The wash buffer (50 mM of NaH₂PO₄ buffer, pH 7.0) was applied on top of the column and the eluent fractions were collected individually. The collected fractions were analyzed on SDS-PAGE gel (containing 10% acrylamide). The protein fractions were combined together and buffer was exchanged against the storage buffer (50 mM KH₂PO₄ buffer pH 7.0 containing 10 mM NaCl, 1 mM MgCl₂) with the desalting column as described in earlier section. The purified enzymes were stored at 4° C. until further use.

### Example 5: Protein Concentration Measurement Using BCA Assay

The concentration of purified lactases was determined using Pierce™ BCA protein assay kit (Thermo Fisher Scientific, Germany) by following the protocol supplied with the kit.

### Example 6: Activity Determination Using Purified Enzymes on Lactose as Substrate at pH 6.7 at 37° C.

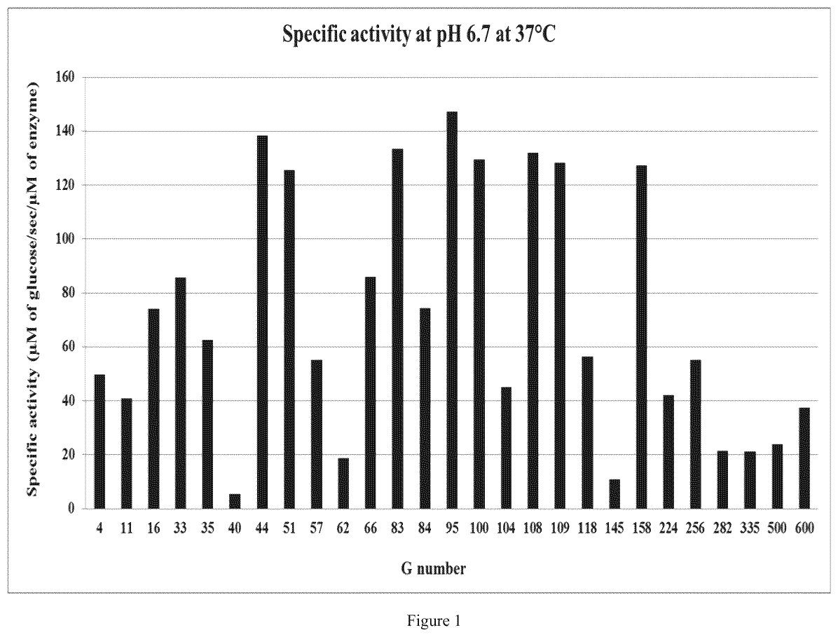

To measure the beta-galactosidase activity, the purified lactases were diluted to 40× in buffer A (50 mM NaH₂PO₄ buffer pH 6.7 containing 100 μM of MgSO₄). In a separate reaction, the diluted enzyme was incubated with lactose solution prepared in buffer B (140 mM of lactose prepared in 100 mM sodium-citrate buffer of pH 6.7, containing 100 μM of MgSO₄). The reaction mixture was prepared by mixing 13 μL of diluted enzyme and 37 μL of lactose solution in a PCR tube. The reaction mixture was incubated in a DNA thermal cycler with the following incubation parameters (reaction time; 10 min at 37° C., enzyme inactivation; 10 min at 95° C., cooling; 4° C.). The reaction mixtures were stored at −20° C. until further use. To determine the amount of glucose formed during the reaction, 10 μL of the reaction mixture was transferred to one well of standard microtiter plate (Thermo Fischer Scientific, Denmark) containing 80 μL of buffer C (100 mM of NaH₂PO₄ buffer, pH 7.0, containing glucose oxidase; 0.6 g/L (Sigma Aldrich), 2,2′-azino-bis(3-ethylbenzothiazoline-6-sulfonic acid diammonium salt); ABTS: 1.0 g/L (Sigma Aldrich), horseradish peroxidase; 0.02 g/L (Sigma Adrich)) and incubated at 30° C. for 40 min. After 40 min, the absorbance was determined at 610 nm using FLUOStar Omega UV-plate reader (BMG Labtech, Germany). The absorbance values between 0.1 and 1.5 were used for calculations, if the A610 nm value >1.5, the reaction mixture was diluted up to 10× with buffer A. With each purified enzyme, the reactions were carried out in triplicate and the mean value of the triplicate measurement was used for calculation. The protein purification performed with the E. coli cells transformed with the empty pBAD/His was used for normalization. Using a known concentration of glucose (0-2.5 mM), a standard curve was drawn and the slope of the curve was used to calculate the glucose formed during the reaction. The maximum absorbance value for each lactase was used to determine μM of glucose formed per sec, described as 1 Unit of Activity with Lactose at pH 6.7 at 37° C. (UAL-1). The specific activity at pH 6.7 at 37° C. is defined as μM of glucose formed per second per μM of enzyme (μM of glucose/sec/μM of enzyme) and determined by dividing UAL-1 by the protein concentration in μM, described as SUAL-1. The specific activity of SEQ ID NO:34 and SEQ ID NO:35 were determined under the similar conditions. The high specific activity at pH 6.7 is highly desired for robustness for the enzyme in fresh and fermented milk applications. The detail results of the specific activity of enzymes at pH 6.7 at 37° C. are described in FIG. 1.

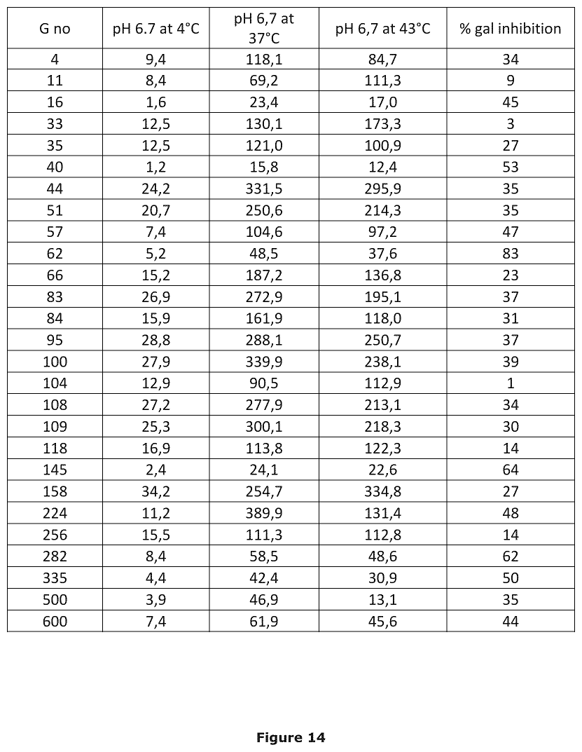

Additionally the activity was described as μmole of glucose formed per minute per milligram of enzyme added. The results are shown in FIG. 14.

### Example 7: Activity determination using purified enzymes in the presence of galactose at pH 6.7 at 37° C.

The purified lactases were diluted to 40× in buffer A (50 mM NaH₂PO₄ buffer pH 6.7 containing 100 μM of MgSO₄). In separate reactions, the diluted enzymes were incubated with buffer D (140 mM of lactose and 140 mM of galactose prepared in 100 mM sodium-citrate buffer of pH 6.7, containing 100 μM of MgSO₄). The reaction mixture consists of 13 μL of the diluted enzyme and 37 μL of buffer D in a PCR tube. The reaction mixture was incubated in thermal cycler with the following incubation parameters (reaction time: 10 min at 37° C., enzyme inactivation: 10 min at 95° C., cooling: 4° C.). The reaction mixtures were stored at −20° C. until further use. To determine the amount of glucose formed during the reaction, 10 μL of the reaction mixture was transferred to one well of standard microtiter plate (Thermo Fischer Scientific, Denmark) containing 80 μL of buffer C (100 mM of NaH2PO4 buffer, pH 7.0, containing glucose oxidase; 0.6 g/L (Sigma Aldrich), 2,2′-azino-bis (3-ethylbenzothiazoline-6-sulfonic acid diammonium salt); ABTS: 1.0 g/L (Sigma Aldrich), horseradish peroxidase; 0.02 g/L (Sigma Adrich)) and incubated at 30° C. for 40 min. After 40 min, the absorbance was determined at 610 nm using FLUOStar Omega UV-plate reader (BMG Labtech, Germany). The absorbance values between 0.1 and 1.5 were used for calculations, if the A610 nm value >1.5, the reaction mixture was diluted up to 10× with buffer A. With each purified enzyme, the reactions were carried out in triplicate and the mean value of the triplicate measurement was used for calculation. The protein purification performed with the E. coli cells transformed with the empty pBAD/His was used for normalization. Using a known concentration of glucose (0-2.5 mM), a standard curve was drawn and the slope of the curve was used to calculate the absorbance corresponding to 1 μM of glucose formed during the reaction. The maximum absorbance value for each lactase was used to determine μM of glucose formed per sec, described as 1 Unit of Activity with Galactose at pH 6.7 at 37° C. (UAG). The specific activity at pH 6.7 at 37° C. in presence of galactose is defined as μM of glucose formed per second per μM of enzyme (μM of glucose/sec/μM of enzyme) and determined by dividing UAG by the protein concentration in μM, described as SUAG.

The percentage inhibition of enzymes with galactose is calculated by using the formula

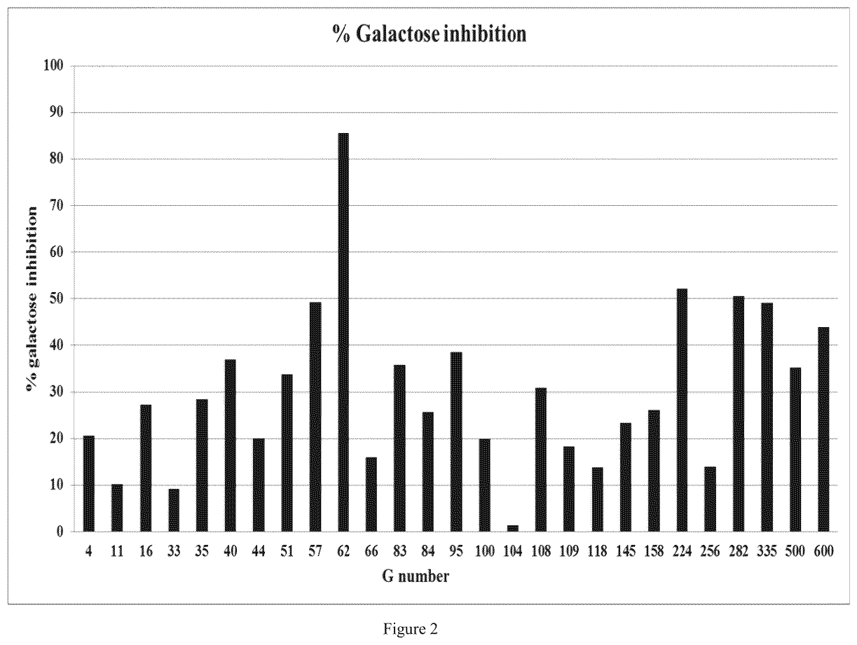

% inhibition=100*(A−B)/A

Where A is specific activity in of enzymes with lactose at pH 6.7 at 37° C. (SUAL) as described in the example 6, and B stand for the specific activity of enzymes in presence of galactose at pH 6.7 at 37° C. (SUAG) as described in the example 7. The detail results of the % galactose inhibition are described the FIG. 2 and FIG. 14. The lower galactose inhibition is highly relevant for the applications where very low lactose concentration is desired. Additionally the activity was described as μmole of glucose formed per minute per milligram of enzyme added. The results are shown in FIG. 14.

Note: relatively high standard deviations in galactose inhibition measurement are due to trace amounts of glucose impurities in purchased galactose.

### Example 8: Activity Determination Using Purified Enzymes on Lactose as Substrate at pH 6.7 at 4° C.

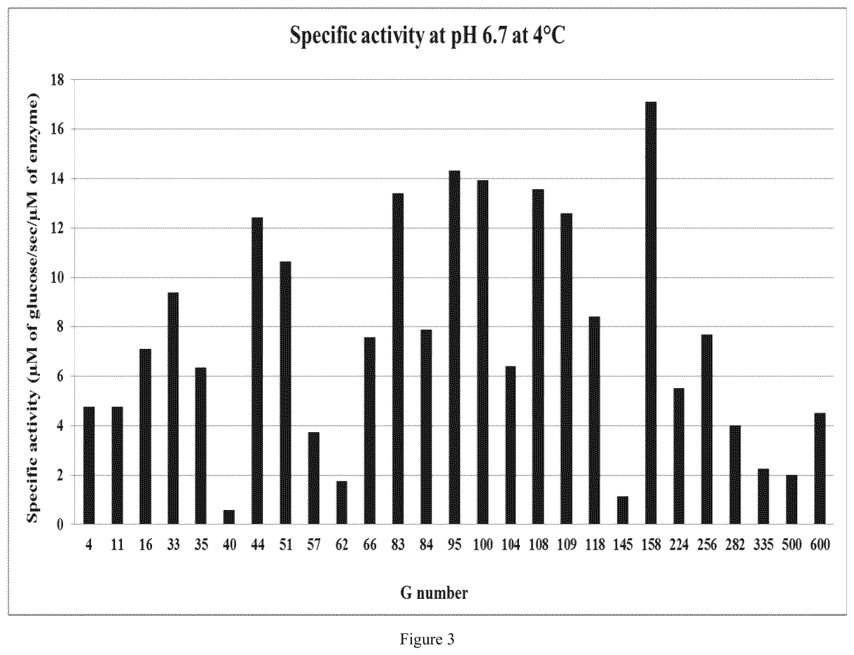

The purified lactases were diluted up to 40× in buffer A (50 mM NaH₂PO₄ buffer pH 6.7 containing 100 μM of MgSO₄). In a separate reaction, the diluted enzyme was incubated with lactose solution prepared in buffer B (140 mM of lactose prepared in 100 mM sodium-citrate buffer of pH 6.7, containing 100 μM of MgSO₄). The reaction mixture was prepared by mixing 13 μL of diluted purified enzyme and 37 μL of lactose solution in a PCR tube. The reaction mixture was incubated in a DNA thermal cycler using the following incubating parameters (reaction time; 60 min at 4° C., enzyme inactivation; 10 min at 95° C., storage; 4° C.). The reaction mixtures were stored at −20° C. freezer until further use. The amount of glucose formed during the reaction was determined by following the protocol described in example 6. The maximum absorbance value for each lactase was used to determine μM of glucose formed per sec, described as 1 Unit of Activity with Lactose at pH 6.7 at 4° C. (UAL-2). The specific activity at pH 6.7 at 4° C. is defined as μM of glucose formed per second per μM of enzyme (μM of glucose/sec/μM of enzyme), and is determined by dividing UAL-2 by the protein concentration in μM, described as SUAL-2. The high specific activity at pH 6.7 at 4° C. is highly desired for the lactose hydrolysis for fresh/pasteurized milk applications. The detail results of the specific activity of enzymes at pH 6.7 at 4° C. are described in the FIG. 3. Additionally the activity was described as μmole of glucose formed per minute per milligram of enzyme added. The results are shown in FIG. 14.

### Example 9: Activity determination using purified enzymes on lactose as substrate at pH 6.7 at 43° C.

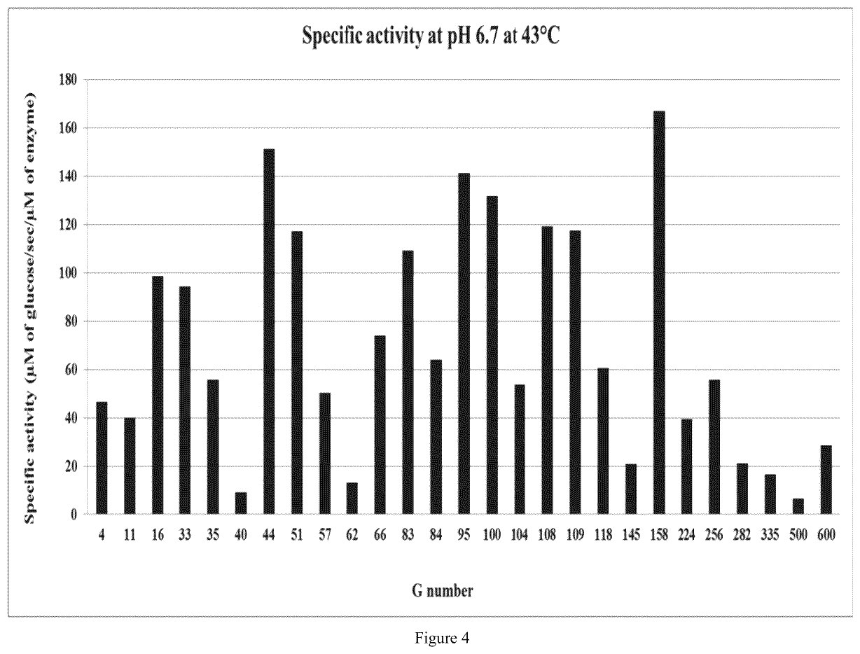

The purified lactases were diluted to 40× in buffer A (50 mM NaH₂PO₄ buffer pH 6.7 containing 100 μM of MgSO₄). In a separate reaction, the diluted enzyme was incubated with lactose solution prepared in buffer B (140 mM of lactose prepared in 100 mM sodium-citrate buffer of pH 6.7, containing 100 μM of MgSO₄). The reaction mixture was prepared by mixing 13 μL of diluted purified enzyme and 37 μL of lactose solution in a PCR tube. The reaction mixture was incubated in a DNA thermal cycler using the following incubating parameters (reaction time; 10 min at 43° C., enzyme inactivation; 10 min at 95° C., storage; 4° C.). The reaction mixtures were stored at −20° C. freezer until further use. The amount of the glucose formed during the reaction was determined by following the protocol described in example 6. The maximum absorbance value for each lactase was used to determine μM of glucose formed per sec, described as 1 Unit of Activity with Lactose at pH 6.7 at 43° C. (UAL-3). The specific activity at pH 6.7 at 43° C. is defined as μM of glucose formed per second per μM of enzyme (μM of glucose/sec/μM of enzyme), and is determined by dividing UAL-3 by the protein concentration in μM, described as SUAL-3. The high specific activity at pH 6.7 at 43° C. is highly desired for the lactose hydrolysis for the fermented milk applications. The detail results of the specific activity of enzymes at pH 6.7 at 43° C. are described in FIG. 4. Additionally the activity was described as μmole of glucose formed per minute per milligram of enzyme added. The results are shown in FIG. 14.

### Example 10: Activity Determination Using purified Enzymes on Lactose as Substrate at pH 5.5 at 4° C.

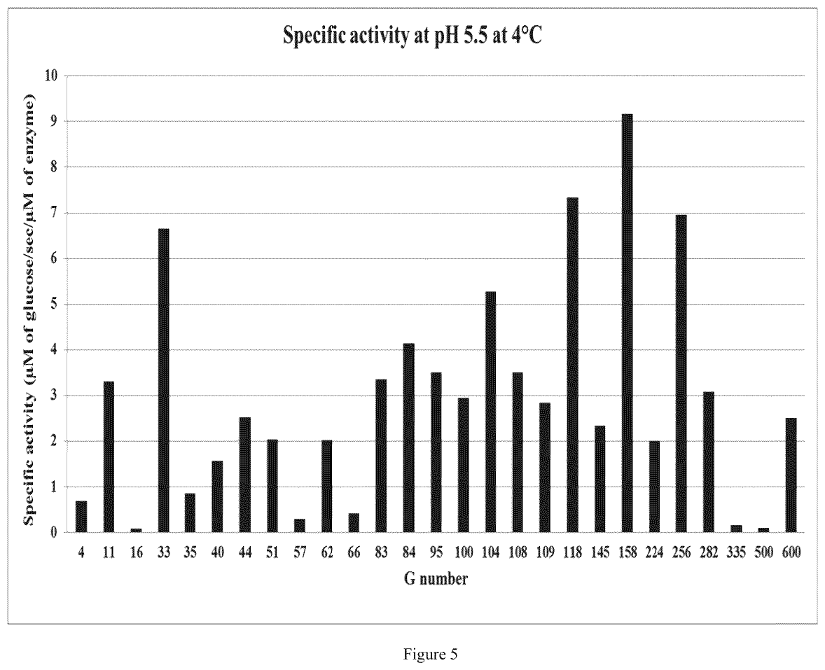

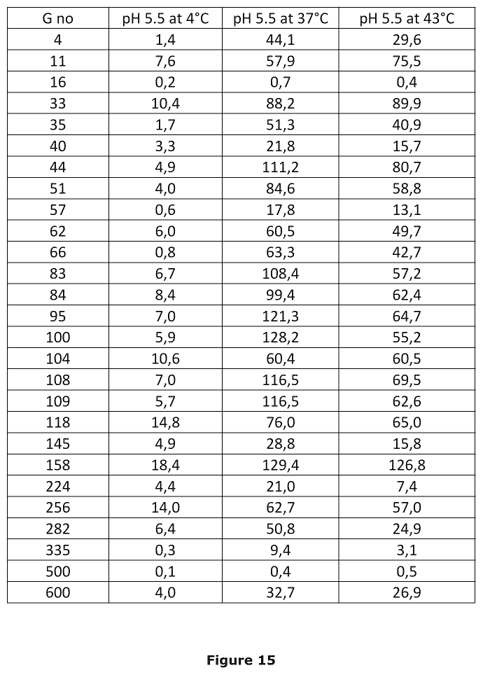

The purified lactases were diluted up to 40× in buffer A (50 mM NaH₂PO₄ buffer pH 6.7 containing 100 μM of MgSO₄). In a separate reaction, the diluted enzyme was incubated with lactose solution prepared in buffer E (140 mM of lactose prepared in 100 mM sodium-citrate buffer of pH 5.5, containing 100 μM of MgSO₄). The reaction mixture was prepared by mixing 13 μL of diluted purified enzyme and 37 μL of lactose solution in a PCR tube. The reaction mixture was incubated in a DNA thermal cycler using the following incubating parameters (reaction time; 60 min at 4° C., enzyme inactivation; 10 min at 95° C., storage; 4° C.). The substrate solution was prepared in a buffer of pH 5.5 and enzyme solution had a pH of 6.7. To initiate the reaction, 13 μL of enzyme was added to 37 μL of substrate solution. This mixing of these two buffers eventually increases the reaction pH from 5.5 to 5.7. The reaction mixtures were stored at −20° C. freezer until further use. To determine the amount of glucose formed during the reaction, 10 μL of the reaction mixture was transferred to one well of standard microtiter plate containing 80 μL of buffer C and incubated at 30° C. for 40 min. After 40 min, the absorbance was determined at 610 nm using FLUOStar Omega UV-plate reader (BMG Labtech, Germany). The absorbance value between 0.1 and 1.5 were used for calculations, if the A610 nm value>1.5, the reaction mixture was diluted up to 5× with buffer A. With each purified enzyme, the reactions were carried out in triplicate and the mean value of the triplicate measurement was used for calculations. The maximum absorbance value for each lactase was used to determine μM of glucose formed per sec, described as 1 Unit of Activity with Lactose at pH 5.5 at 4° C. (UAL-4). The specific activity at pH 5.5 at 4° C. is defined as μM of glucose formed per second per μM of enzyme (μM glucose/sec/μM of enzyme), and is determined by dividing UAL-4 by the protein concentration in μM, described as SUAL-4. The high specific activity at pH 5.5 at 4° C. is relevant for the lactose hydrolysis in the fermented milk applications. The detail results of the specific activity of enzymes at pH 5.5 at 4° C. are described in the FIG. 5. Additionally the activity was described as μmole of glucose formed per minute per milligram of enzyme added. The results are shown in FIG. 15.

### Example 11: Activity Determination Using Purified Enzymes on Lactose as Substrate at pH 5.5 at 37° C.

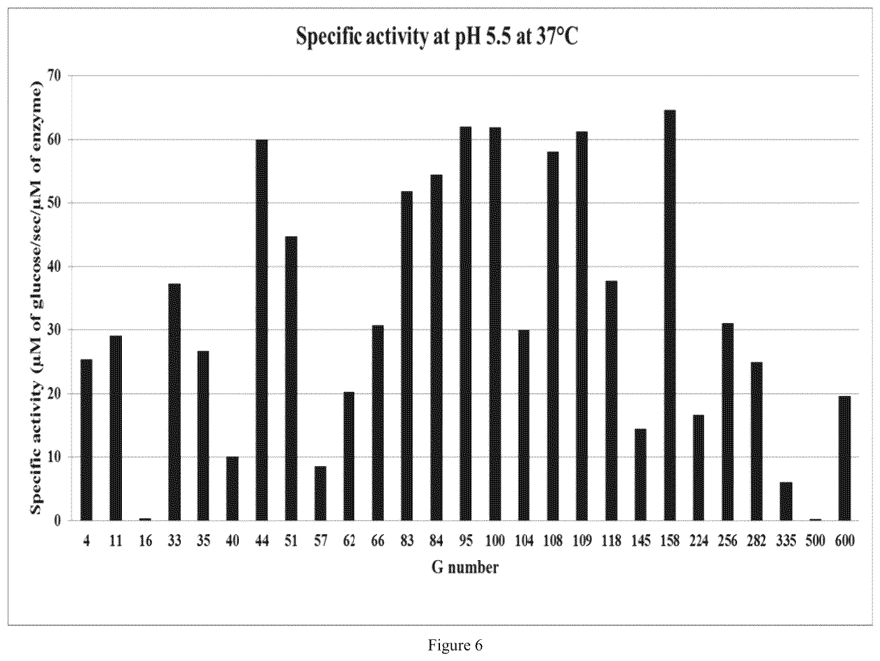

The purified lactases were diluted up to 40× in buffer A (50 mM NaH₂PO₄ buffer pH 6.7 containing 100 μM of MgSO₄). In a separate reaction, the diluted enzyme was incubated with lactose solution prepared in buffer E (140 mM of lactose prepared in 100 mM sodium-citrate buffer of pH 5.5, containing 100 μM of MgSO₄). The reaction mixture was prepared by mixing 13 μL of diluted purified enzyme and 37 μL of lactose solution in a PCR tube. The substrate solution was prepared in a buffer of pH 5.5 and enzyme solution had a pH of 6.7. To initiate the reaction, 13 μL of enzyme was added to 37 μL of substrate solution. This mixing of these two buffers eventually increases the reaction pH from 5.5 to 5.7. The reaction mixture was incubated in a DNA thermal cycler using the following incubating parameters (reaction time; 10 min at 37° C., enzyme inactivation; 10 min at 95° C., storage; 4° C.). The reaction mixtures were stored at −20° C. until further use. The amount of glucose formed during the reaction was determined by following the protocol as described in the example 10. The maximum absorbance value for each lactase was used to determine μM of glucose formed per sec, described as 1 Unit of Activity with Lactose at pH 5.5 at 37° C. (UAL-5). The specific activity at pH 5.5 at 37° C. is defined as μM of glucose formed per second per μM of enzyme (μM of glucose/sec/μM of enzyme), and is determined by dividing UAL-5 by the protein concentration in μM, described as SUAL-5. The high specific activity at pH 5.5 at 37° C. is relevant for the lactose hydrolysis in the fermented milk applications and sweet whey lactose hydrolysis. The detail results of the specific activity of enzymes at pH 5.5 at 37° C. are described in the FIG. 6. Additionally the activity was described as μmole of glucose formed per minute per milligram of enzyme added. The results are shown in FIG. 15.

### Example 12: Activity Determination Using Purified Enzymes on Lactose as Substrate at pH 5.5 at 43° C.

The purified lactases were diluted up to 40× in buffer A (50 mM NaH₂PO₄ buffer pH 6.7 containing 100 μM of MgSO₄). In a separate reaction, the diluted enzyme was incubated with lactose solution prepared in buffer E (140 mM of lactose prepared in 100 mM sodium-citrate buffer of pH 5.5, containing 100 μM of MgSO₄). The reaction mixture was prepared by mixing 13 μL of diluted purified enzyme and 37 μL of lactose solution in a PCR tube. The substrate solution was prepared in a buffer of pH 5.5 and enzyme solution had a pH of 6.7. To initiate the reaction, 13 μL of enzyme was added to 37 μL of substrate solution. This mixing of these two buffers eventually increases the reaction pH from 5.5 to 5.7.

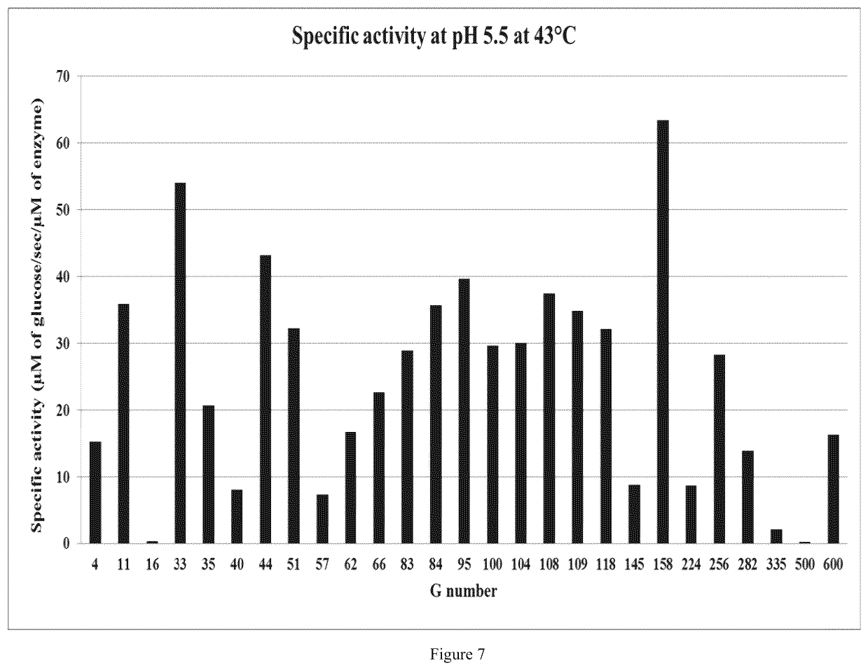

The reaction mixture was incubated in a DNA thermal cycler using the following incubating parameters (reaction time; 10 min at 43° C., enzyme inactivation; 10 min at 95° C., storage; 4° C.). The reaction mixtures were stored at −20° C. until further use. The amount of glucose formed during the reaction was determined by following the protocol described in the example 10. The maximum absorbance value for each lactase was used to determine μM of glucose formed per sec, described as 1 Unit of Activity with Lactose at pH 5.5 at 43° C. (UAL-6). The specific activity at pH 5.5 at 43° C. is defined as μM of glucose formed per second per μM of enzyme (μM of glucose/sec/μM of enzyme), and is determined by dividing UAL-6 by the protein concentration in μM, described as SUAL-6. The high specific activity at pH 5.5 at 43° C. is relevant for the lactose hydrolysis in the fermented milk applications and sweet whey lactose hydrolysis. The detail results of the specific activity of enzymes at pH 5.5 at 43° C. are described in the FIG. 7. Additionally the activity was described as μmole of glucose formed per minute per milligram of enzyme added. The results are shown in FIG. 15.

### Example 13: Activity Determination Using Purified Enzymes on Lactose as Substrate at pH 4.5 at 4° C.

The purified lactases were diluted up to 40× in buffer A (50 mM NaH₂PO₄ buffer pH 6.7 containing 100 μM of MgSO₄). In a separate reaction, the diluted enzyme was incubated with lactose solution prepared in buffer F (140 mM of lactose prepared in 100 mM sodium-citrate buffer of pH 4.5, containing 100 μM of MgSO₄). The reaction mixture was prepared by mixing 13 μL of diluted purified enzyme and 37 μL of lactose solution in a PCR tube. The substrate solution was prepared in a buffer of pH 4.5 and enzyme solution had a pH of 6.7. To initiate the reaction, 13 μL of enzyme was added to 37 μL of substrate solution. This mixing of these two buffers eventually increases the reaction pH from 4.5 to 4.7.

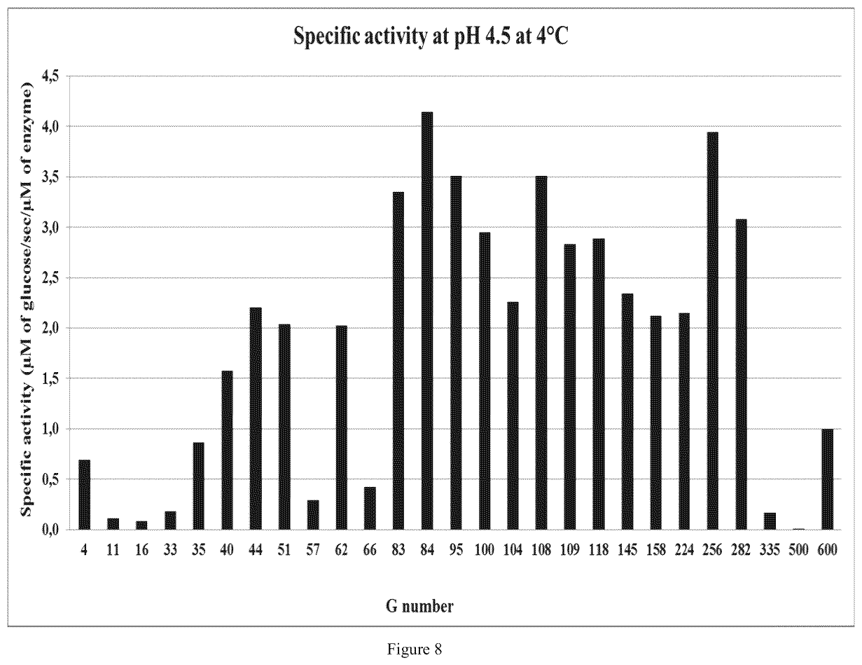

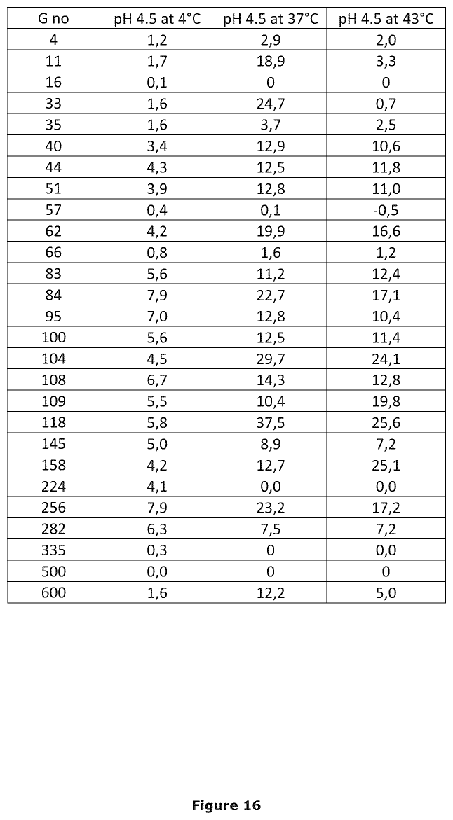

The reaction mixture was incubated in a DNA thermal cycler using the following incubating parameters (reaction time; 60 min at 4° C., enzyme inactivation; 10 min at 95° C., storage; 4° C.). To determine the amount of glucose formed during the reaction, 10 μL of the reaction mixture was transferred to one well of standard microtiter plate containing 80 μL of buffer C (as described in example 6) and incubated at 30° C. for 40 min. After 40 min, the absorbance was determined at 610 nm using FLUOStar Omega UV-plate reader. The absorbance value between 0.1 and 1.5 were used for calculations, if the A610 nm value >1.5, the reaction mixture was diluted up to 5× with buffer A. With each purified enzyme, the reactions were carried out in triplicate and the mean value of the triplicate measurement was used for calculation. The maximum absorbance value for each lactase was used to determine μM of glucose formed per sec, described as 1 Unit of Activity with Lactose at pH 4.5 at 4° C. (UAL-7). The specific activity at pH 4.5 at 4° C. is defined as μM of glucose formed per second per μM of enzyme (μM of glucose/sec/μM of enzyme), and is determined by dividing UAL-7 by the protein concentration in μM, described as SUAL-7. The high specific activity at pH 4.5 at 4° C. is relevant for the lactose hydrolysis in the fermented milk applications. The detail results of the specific activity of enzymes at pH 4.5 at 4° C. are described in the FIG. 8. Additionally the activity was described as μmole of glucose formed per minute per milligram of enzyme added. The results are shown in FIG. 16.

### Example 14: Activity Determination Using Purified Enzymes on Lactose as Substrate at pH 4.5 at 37° C.

The purified lactases were diluted up to 40× in buffer A (50 mM NaH2PO4 buffer pH 6.7 containing 100 μM of MgSO₄). In a separate reaction, the diluted enzyme was incubated with lactose solution prepared in buffer F (140 mM of lactose prepared in 100 mM sodium-citrate buffer of pH 4.5, containing 100 μM of MgSO₄). The reaction mixture was prepared by mixing 13 μL of diluted purified enzyme and 37 μL of lactose solution in a PCR tube. The substrate solution was prepared in a buffer of pH 4.5 and enzyme solution had a pH of 6.7. To initiate the reaction, 13 μL of enzyme was added to 37 μL of substrate solution. This mixing of these two buffers eventually increases the reaction pH from 4.5 to 4.7.

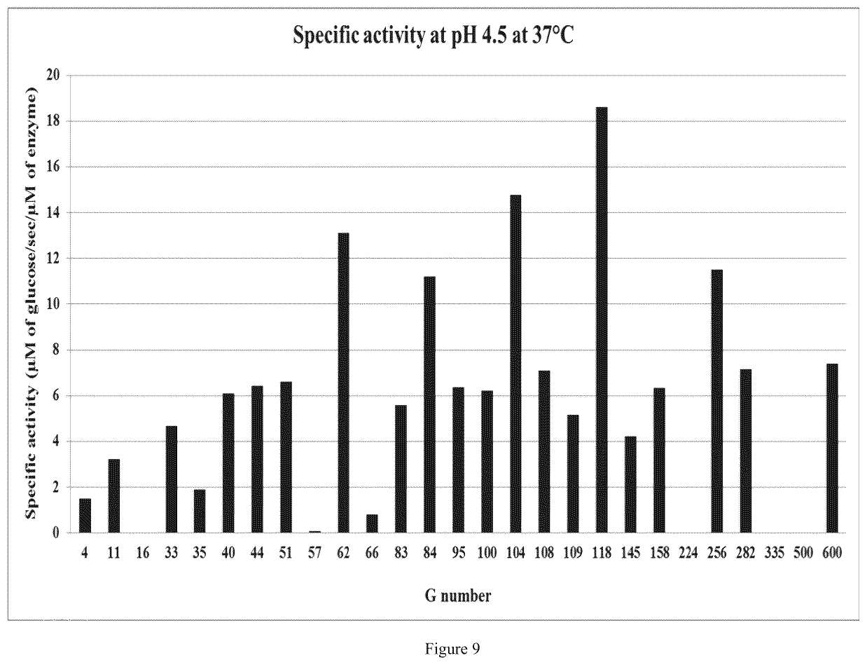

The reaction mixture was incubated in a DNA thermal cycler using the following incubating parameters (reaction time; 10 min at 37° C., enzyme inactivation; 10 min at 95° C., storage; 4° C.). The reaction mixtures were stored at −20° C. until further use. The amount of glucose formed during the reaction was determined by following the protocol described in the example 13. The maximum absorbance value for each lactase was used to determine μM of glucose formed per sec, described as 1 Unit of Activity with Lactose at pH 4.5 at 37° C. (UAL-8). The specific activity at pH 4.5 at 37° C. is defined as μM of glucose formed per second per μM of enzyme (μM of glucose/sec/μM of enzyme), and is determined by dividing UAL-8 by the protein concentration in μM, described as SUAL-8. The high specific activity at pH 4.5 at 37° C. is relevant for the lactose hydrolysis in the fermented milk applications and acidic whey lactose hydrolysis. The detail results of the specific activity of enzymes at pH 4.5 at 37° C. are described in the FIG. 9. Additionally the activity was described as μmole of glucose formed per minute per milligram of enzyme added. The results are shown in FIG. 16.

### Example 15: Activity Determination Using Purified Enzymes on Lactose as Substrate at pH 4.5 at 43° C.

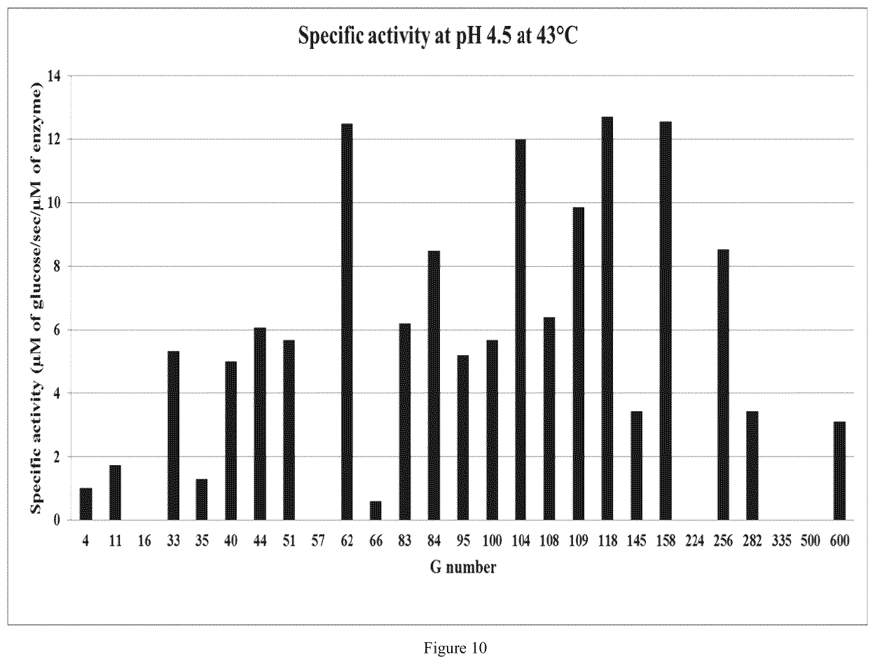

The purified lactases were diluted up to 40× in buffer A (50 mM NaH₂PO₄ buffer pH 6.7 containing 100 μM of MgSO₄). In a separate reaction, the diluted enzyme was incubated with lactose solution prepared in buffer F (140 mM of lactose prepared in 100 mM sodium-citrate buffer of pH 4.5, containing 100 μM of MgSO₄). The reaction mixture was prepared by mixing 13 μL of diluted purified enzyme and 37 μL of lactose solution in a PCR tube. The substrate solution was prepared in a buffer of pH 4.5 and enzyme solution had a pH of 6.7. To initiate the reaction, 13 μL of enzyme was added to 37 μL of substrate solution. This mixing of these two buffers eventually increases the reaction pH from 4.5 to 4.7. The reaction mixture was incubated in a DNA thermal cycler using the following incubating parameters (reaction time; 10 min at 43° C., enzyme inactivation; 10 min at 95° C., storage; 4° C.). The reaction mixtures were stored at −20° C. until further use. The amount of glucose formed during the reaction was determined by following the protocol described in the example 13. The maximum absorbance value for each lactase was used to determine μM of glucose formed per sec, described as 1 Unit of Activity with Lactose at pH 4.5 at 43° C. (UAL-9). The specific activity at pH 4.5 at 43° C. is defined as μM of glucose formed per second per μM of enzyme (μM of glucose/sec/μM of enzyme), and is determined by dividing UAL-9 by the protein concentration in μM, described as SUAL-9. The high specific activity at pH 4.5 at 43° C. is relevant for the lactose hydrolysis in the fermented milk applications and acidic whey lactose hydrolysis. The detail results of the specific activity of enzymes at pH 4.5 at 43° C. are described in the FIG. 10. Additionally the activity was described as μmole of glucose formed per minute per milligram of enzyme added. The results are shown in FIG. 16.

### Example 16: Activity Determination in BLU Units

The commercially available NOLA™ Fit enzyme (Chr-Hansen, Denmark) was diluted in a range from 0.5 BLU/mL to 2.5 BLU/mL in buffer G (50 mM NaH₂PO₄ buffer pH 7.0 containing 100 μM of MgSO₄, 0.045% Brij, Sigma Aldrich). The diluted enzyme was incubated with lactose solution prepared in buffer H (105 mM of lactose prepared in 100 mM sodium-citrate buffer of pH 6.7, containing 100 μM of MgSO₄). The reaction mixture was prepared by mixing 13 μL of diluted purified enzyme and 37 μL of lactose solution in a PCR tube. The reaction mixture was incubated in a DNA thermal cycler using the following incubating parameters (reaction time; 10 min at 37° C., enzyme inactivation; 10 min at 95° C., storage; 4° C.). The amount of glucose conversion was determined by transferring 10 μL of the reaction mixture in a single well of standard microtiter plate containing 80 μL of buffer C and incubated at 30° C. for 40 min. After 40 min, the absorbance was determined at 610 nm using FLUOStar Omega UV-plate reader (BMG Labtech, Germany). The measured absorbance values were used to draw a standard curve against BLU/mL. The maximum slope of the curve was used to determine the activity of new enzymes in BLU/mL.

### Example 17: Activity Determination of New Lactases in BLU/mL Using Lactose as Substrate

The purified lactases were diluted up to 50× in buffer A (50 mM NaH₂PO₄ buffer pH 6.7 containing 100 μM of MgSO₄). In a separate reaction, the diluted enzyme was incubated with lactose solution prepared in buffer H (105 mM of lactose prepared in 100 mM sodium-citrate buffer of pH 6.7, containing 100 μM of MgSO₄). The reaction mixture was prepared by mixing 13 μL of diluted purified enzyme and 37 μL of lactose solution in a PCR tube. The reaction mixture was incubated in a DNA thermal cycler using the following incubating parameters (reaction time; 10 min at 37° C., enzyme inactivation; 10 min at 95° C., storage; 4° C.). After the reaction, 10 μL of the reaction mixture was transferred to one well of standard microtiter plate containing 80 μL of buffer C (as described in example 6) and incubated at 30° C. for 40 min. After 40 min, the absorbance was determined at 610 nm using FLUOStar Omega UV-plate reader. The absorbance value between 0.1 and 1.5 were used for calculations, if the A610 nm value >1.5, the reaction mixture was diluted up to 5× with buffer A. The maximum absorbance values were used to calculate the enzyme activity in BLU/mL, using standard curve described in example 16.

### Example 18: Percentage Residual Lactose Measurement in Fresh Milk at Cold Temperature

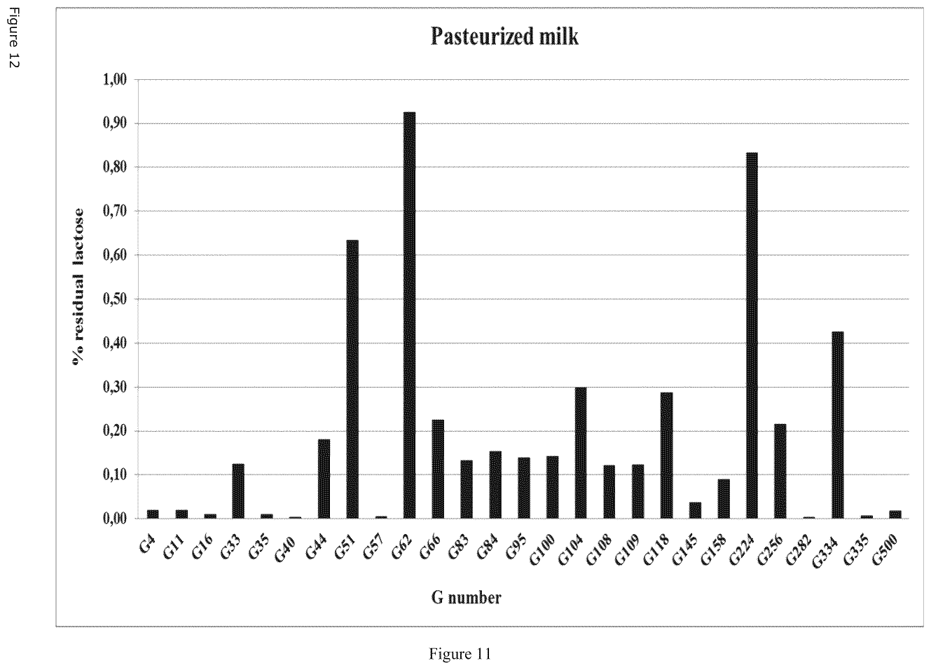

2 mL of commercial pasteurized milk (1.5% Fat pasteurized milk, Arla Food) was mixed with 10-125 μL of enzyme (equivalent to 10 BLU/mL) as determined in the example 17, in 10 mL glass tube. The samples were incubated under constant conditions for 24 hours at 4° C. After the incubation, the reaction was stopped by heat inactivation at 95° C. for 7 min, followed by storage at −20° C. until further use. The amount of remaining lactose in the milk was analyzed using an HPLC assay. Samples for analysis were treated with 1.8 mL protein precipitation solution (0.083 M PCA and 2 mM Na-EDTA) and 2 mL of MQW prior to centrifugation at 2800 rpm for 30 min at 4° C. An aliquot of the supernatant was diluted a total of 200-fold using a Janus dilution robot (PerkinElmer, Waltham, MA, USA). The diluted samples were analyzed on a Dionex ICS-5000 system (Thermo Fischer Scientific, Waltham (MA), USA) using 4×250 mm CarboPac SA20 analytical column (Thermo Fischer Scientific, Waltham, MA, USA) and a pulsed amperometric detector. The detector was set to a simple three-step potential waveform, selective for detection of carbohydrates. The eluent was set to 1 mM KOH and was continuously regenerated through a trap column (CR-TC, Thermo Fischer Scientific, Waltham (MA), USA). The flow rate of the eluent was 1.2 mL/min and the analysis time was 10 min per injection. The lactose in each sample was quantified using a three-point external calibration curve prepared by adding known amounts of lactose monohydrate (Sigma-Aldrich, St. Louis, MO, USA) to MQW. Concentrations were calculated based on the chromatographic peak heights. The measured percentage residual lactose in fresh milk is shown in FIG. 11.

### Example 19: Activity Determination in UHT Milk at Room Temperature

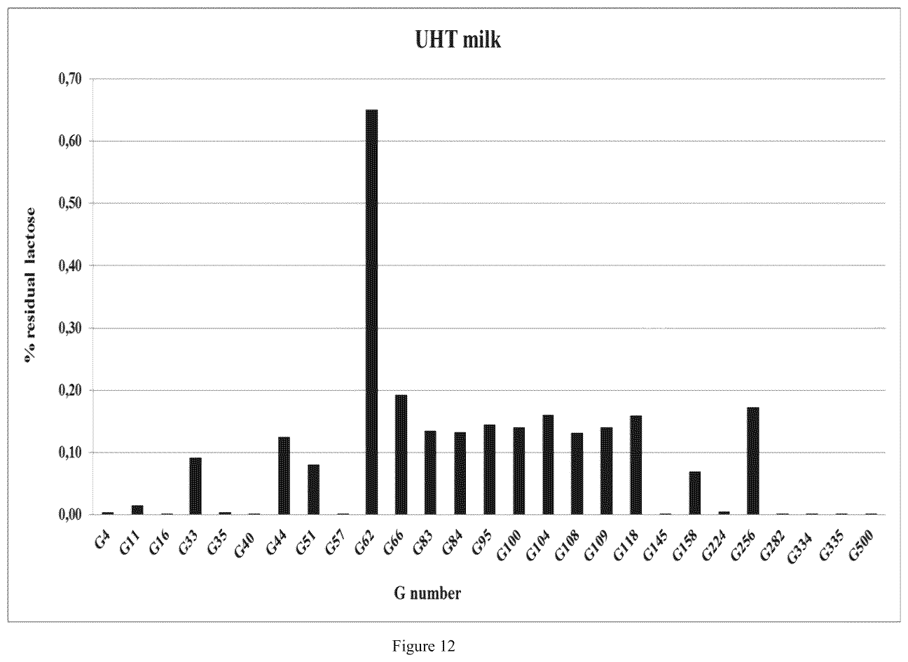

2 mL of UHT milk (1.5% Fat UHT milk, Arla Food) was mixed with 2-25 μL of enzyme (equivalent to 2 BLU/mL) as determined in example 17, in 10 mL glass tube. The samples were incubated under constant conditions for 24 hours at 25° C. After the incubation, the reaction was stopped by heat inactivation at 95° C. for 7 min, followed by storage at −20° C. until further use. The amount of residual lactose in UHT milk was analyzed using HPLC by following the protocol as described in example 18. The percentage of residual lactose in fresh milk after hydrolysis is listed in the FIG. 12.

### Example 20: Enzyme performance at high temperature in buffer

The purified enzyme was diluted to 5 BLU/mL in buffer A (50 mM NaH2PO4 buffer pH 6.7 containing 100 μM of MgSO₄). In a separate reaction, 13 μL of the diluted enzyme was incubated in a DNA thermal cycler with lactose solution (105 mM lactose prepared in 100 mM sodium-citrate buffer of pH 6.7, containing 100 μM of MgSO₄). The reaction mixture was prepared by mixing 13 μL of enzyme and 37 μL of lactose solution in a PCR tube. The reaction mixture was incubated in a DNA thermal cycler using the following incubating parameters (reaction time; 10 min at 37° C., enzyme inactivation; 10 min at 95° C., storage; 4° C.). After the reaction, 10 μL of the reaction mixture was transferred to one well of standard microtiter plate containing 80 μL of buffer C (as described in example 6) and incubated at 30° C. for 40 min. After 40 min, the absorbance was determined at 610 nm using FLUOStar Omega UV-plate reader. The absorbance value between 0.1 and 1.5 were used for calculations, if the A610 nm value >1.5, the reaction mixture was diluted up to 5× with buffer A. The measured absorbance was called Abs37° C., and considered as reference value for calculations.

To measure the impact of heat treatment on enzyme activity, in a separate reaction, 13 μL of the diluted enzyme (5 BLU/mL) was incubated in a DNA thermal cycler using the following incubating parameter (at 72° C. for 15 sec or 74° C. for 15 sec or 76° C. for 6 sec or 78° C. for 6 sec or 80° C. for 4 sec or 85° C. for 5 sec or 90° C. for 5 sec or 95° C. for 5 sec, followed by storage at 4° C.). The activity of the heat treated enzyme was determined by incubation with the lactose solution (105 mM lactose prepared in 100 mM sodium-citrate buffer of pH 6.7, containing 100 μM of MgSO₄), as described above. The measured absorbance at different temperature (for example at 72° C., 74° C., 76° C., 78° C., 80° C., 85° C., 90° C. or 95° C.) was called as Abs72° C., Abs74° C., Abs76° C., Abs78° C., Abs80° C., Abs85° C., Abs90° C., Abs95° C.

The percentage residual activity at high temperature was determined using the formula,

% residual activity=(Abs72° C./Abs37° C.)*100

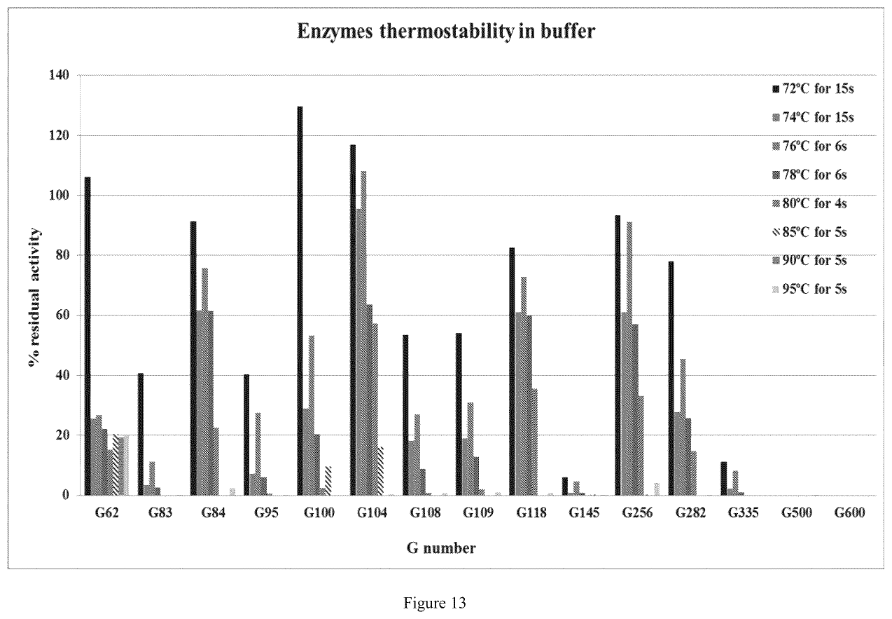

The percentages residual activities of different enzymes at different temperature are described in FIG. 13.

### Example 21: Percentage Residual Lactose after the High Heat Treatment

The effect of heat treatment on the enzyme performance in pasteurized milk was determined by incubating a fixed amount of enzyme in the milk followed by a heat treatment. In separate reactions, 50 μL of the pasteurized milk was mixed with 10 BLU/mL of purified enzyme (as determined in example 17), in a PCR tube. The milk sample was incubated at 72° C. for 15 or 76° C. for 10 sec or 85° C. for 5 sec and 90° C. for 5 sec, followed by incubation at 5° C. for 24 h. After 24 h at 5° C., the reaction was stopped by heating the reaction at 95° C. for 7 min, followed by storage at −20° C. The residual lactose was measured by using the LactoSens® assay kit (Chr. Hansen, Denmark), by following the supplied protocol. The measured residual lactose was determined in g/L was determined at different temperature. The detection limit of the LactoSens® kit is between 0.2 g/L to 10 g/L. The results are described in the table 2.

TABLE 2
The percentage residual lactose in the pasteurized milk treated with a fixed amount of the purified enzyme followed by
incubation at different temperature (72° C. for 15 sec, 76° C.
for 10 sec, 85° C. for 5 sec and 90° C. for 5 sec followed by incubation at 4 C. for 24 h), determined using LactoSens ®
assay kit. The LactoSens ® kit detection limits are in range of
0.2 g/L to 10 g/L of lactose. Here ND; not determined.

| | Residual | Residual | Residual | Residual | Residual |
|---|---|---|---|---|---|
| G- | lactose at | lactose at | lactose at | lactose at | lactose at |
| num- | 4° C. | 72° C. | 76° C. | 85° C. | 90° C. |
| ber | (g/L) | (g/L) | (g/L) | (g/L) | (g/L) |
| G4 | <0.2 | >10.0 | ND | ND | ND |
| G11 | <0.2 | >10.0 | ND | ND | ND |
| G16 | <0.2 | >10.0 | ND | ND | ND |
| G33 | <0.2 | 4.7 | ND | ND | ND |
| G35 | <0.2 | >10.0 | >10.0 | ND | ND |
| G40 | <0.2 | <0.2 | <0.2 | >10.0 | ND |
| G44 | 0.9 | >10.0 | ND | ND | ND |
| G57 | <0.2 | >10.0 | ND | ND | ND |
| G62 | 8.4 | >10.0 | >10.0 | >10.0 | ND |
| G66 | 0.35 | >10.0 | ND | ND | ND |
| G83 | 0.3 | 2.1 | 6.0 | >10.0 | ND |
| G84 | 0.25 | 0.65 | 0.5 | 7.6 | >10 |
| G95 | 0.3 | 6.0 | 8.6 | >10 | ND |
| G100 | 0.4 | 2.4 | 2.6 | >10.0 | ND |
| G104 | 0.35 | 0.45 | 0.5 | 0.45 | >10 |
| G108 | 0.35 | 1.3 | 1.55 | ND | ND |
| G109 | 0.35 | 1.45 | 3.4 | >10.0 | ND |
| G118 | 0.45 | 0.95 | 0.8 | >10.0 | >10 |
| G158 | <0.2 | 3.9 | >10.0 | ND | ND |
| G256 | 0.3 | 1.0 | 0.75 | 3.4 | >10 |
| G282 | <0.2 | <0.2 | <0.2 | <0.2 | >10 |
| G335 | <0.2 | 0.35 | 8.0 | >10.0 | ND |
| G600 | <0.2 | >10.0 | >10.0 | >10.0 | ND |
| G500 | <0.2 | >10.0 | ND | ND | ND |

1. A peptide exhibiting beta-galactosidase enzyme activity, which has an amino acid sequence represented by SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, or enzymatically active fragments thereof, or an amino acid sequence of any one thereof having not more than 1, 2, 3, 4, 5, 6, 7, 8, 9, 10, 11, 12, 13, 14, 15, 16, 17, 18, 19, 20, 21, or 22 amino acid substitutions, additions or deletions.

2. A dimeric peptide exhibiting beta-galactosidase enzyme activity, which dimeric peptide consist of two peptides having an amino acid sequence represented by SEQ ID NO: 2 and 3, 5 and 6, 20 and 21, 23 and 24, 26 and 27, or 28 and 29; or enzymatically active fragments thereof, or an amino acid sequence of any one thereof having not more than 1, 2, 3, 4, 5, 6, 7, 8, 9, 10, 11, 12, 13, 14, 15, 16, 17, 18, 19, 20, 21, or 22 amino acid substitutions, additions or deletions.

3. A method for reducing the lactose content in a composition containing lactose, such as in a dairy products, comprising the step of contacting said composition with a peptide or dimeric peptide exhibiting beta-galactosidase enzyme activity, which peptide has an amino acid sequence represented by SEQ ID NO:1-33, or which dimeric peptide consist of two peptides having an amino acid sequence represented by SEQ ID NO: 2 and 3, 5 and 6, 20 and 21, 23 and 24, 26 and 27, or 28 and 29; or enzymatically active fragments thereof, or any sequence with at least 80% sequence identity to any one of said sequences or enzymatically active fragments; or a host cell expressing any one of said peptide or dimeric peptide, at a pH ranging from 3-10 and at a temperature ranging from 0° C.-140° C.

4. Use of a peptide or dimeric peptide exhibiting beta-galactosidase enzyme activity, which peptide has an amino acid sequence represented by SEQ ID NO:1-33, or which dimeric peptide consist of two peptides having an amino acid sequence represented by SEQ ID NO: 2 and 3, 5 and 6, 20 and 21, 23 and 24, 26 and 27, or 28 and 29; or a sequence with at least 80% sequence identity to any one of said sequences; or a host cell expressing any one of said peptide or dimeric peptide for producing a dairy product with a reduced lactose content.

5. A method for producing a dairy product the method comprising the steps of
a) providing a milk-based substrate comprising lactose;
b) adding an peptide or dimeric peptide exhibiting beta-galactosidase activity, which peptide has an amino acid sequence represented by SEQ ID NO:1-33; or which dimeric peptide consist of two peptides having an amino acid sequence represented by SEQ ID NO: 2 and 3, 5 and 6, 20 and 21, 23 and 24, 26 and 27, or 28 and 29; or a sequence with at least 80% sequence identity to any one of said sequences to said milk-based substrate comprising lactose; and
c) treating said milk-based substrate with said peptide or dimeric peptide exhibiting beta-galactosidase activity.

6. The method according to claim 5, wherein step c) takes place at a pH within a range of 3-10, such as within a range of 3-9, such as within a range of 3-8, such as within a range of 3-7, such as within a range of 3-6, such as within a range of 3-5, such as within a range of 3-4, such as within a range of 4-10, such as within a range of 4-9, such as within a range of 4-8, such as within a range of 4-7, such as within a range of 4-6, such as within a range of 4-5, such as within a range of 5-10, such as within a range of 5-9, such as within a range of 5-8, such as within a range of 5-7, such as within a range of 5-6, such as within a range of 6-10, such as within a range of 6-9, such as within a range of 6-8, such as within a range of 6-7.

7. The method according to any one of claims 5-6, wherein step c) or a part of step c) takes place at a temperature of not more than about 25° C., such as not more than about 20° C., such as not more than about 18° C., such as not more than about 16° C., such as not more than about 14° C., such as not more than about 12° C., such as not more than about 10° C., such as not more than about 8° C., such as not more than about 7° C., such as not more than about 6° C., such as not more than about 5° C., such as not more than about 4° C., such as not more than about 3° C., such as not more than about 2° C.

8. The method according to any one of claims 5-7, wherein step c) or a part of step c) takes place at a temperature of at least about 25° C., such as at least about 30° C., such as at least about 35° C., such as at least about 40° C., such as at least about 45° C., such as at least about 50° C., such as at least about 55° C., such as at least about 60° C., such as at least about 65° C., such as at least about 70° C., such as at least about 75° C., such as at least about 80° C., such as at least about 85° C., such as at least about 90° C., such as at least about 95° C., such as at least about 100° C., such as at least about 110° C., such as at least about 120° C., such as at least about 130° C., such as at least about 120° C., such as at least about 130° C., such as at least about 135° C., such as at least about 140° C.

9. The method according to any one of claims 5-8, wherein the galactose inhibition of said peptide or dimeric peptide is less than 60%, such as less than 55%, such as less than 50%, such as less than about 45%, such as less than about 40%.

10. The method according to any one of claims 5-9, wherein said dairy product is fermented milk product and said step b) is performed during or prior to fermentation.

11. The method according to claim 10, which method does not require the addition of further enzyme after fermentation.

12. The method according to any one of claims 5-10, wherein said dairy product is fermented milk product and said step b) is performed immediately following fermentation.

13. The method according to any one of claims 5-10, wherein said dairy product is fresh milk and said step b) is performed prior to, in conjunction with, or immediately following a step of pasteurization.

14. The method according to any one of claims 5-10, wherein said dairy product is ultra-heat treatment (UHT) milk and said step b) is performed prior to, in conjunction with, or immediately following a step of ultra-heat treatment.

15. The method according to any one of claims 5-14, wherein step c) is started at a temperature of between 40° C. and 100° C., such as at a temperature of between 50° C. and 100° C. such as at a temperature of between 60° C. and 100° C., such as at a temperature of between 70° C. and 100° C., such as at a temperature of between 80° C. and 100° C., such as at a temperature of between 40° C. and 90° C., such as at a temperature of between 40° C. and 80° C., such as at a temperature of between 40° C. and 70° C., such as at a temperature of between 40° C. and 60° C., such as at a temperature of between 40° C. and 50° C.


SEQUENCE LISTING (41 sequences)

SEQ ID NO: 1 - protein, 1049 aa, Bifidobacterium adolescentis
MADTAELAIVHATTASASWLTDPTVFAANRKPAHSSHRYVIGETSEPKQSLDGEWKVRIEQARNVDVESAPFAAVDFEDGDFGAIEVPGHLQMAGYLKNKYVNIQYPWDGHEDPQAPNIPENNHVAIYRRRFALDAQLARTLENDGTVSLTFHGAATAIYVWLDGTFVGYGEDGFTPSEFDVTEALRNGNGNAADSPEAEHTLTVACYEYSSASWLEDQDFWRLHGLFRTVELAAQPHTHVETVQLEADYTAADTAGTADTAELNAALTLRNSADAMTIESTLRDGDGNVVWESTQACNGEIALNSGKMTNIAPWSAESPTLYTLTVRVVGHDGAIIETVTQKIGFRTFRIENGIMTLNGKRIVFKGADRHEFDAKRGRAITREDMLSDVVFCKRHNINAIRTSHYPNQEYWYDLCDEYGLYLIDETNMETHGTWVANNVERPEDGIPGSRPEWEGACVDRINSMMRRDYNHPSVLIWSLGNESSAGEVFRAMYRHAHTIDPNRPVHYEGSVHMREFEDVTDIESRMYAHADEIERYLNDGSPAHTDGPKKPYISCEYMHAMGNSCGNMDEYTALERYPMYQGGFIWDFIDQAIETKLPDGTTRMCYGGDFGDRPSDYEFSGDGLLFADRTPSPKAQEVKQLYANVKIVVSVDEARITNDNLFVSTGDYRFVLRILADGKPVWSTTRRFDVAAGESASFEVDWPVDDYRSNAEELVLEVSQQLGNACDWAPAGYELAFGQCVVAGAKTTADAVDAAGAPADGTVTLGRWNAGVRGQGREALFSRTQGGMVSYTFGEREFVLRRPSITTFRPLTDNDRGAGHAFERAAWAVAGKYARCVDCAIANRGENAVEATYTYELAIPQRTKVTVRYVADTAGLVSLDVEYPGEKNGDLPTIPAFGIEWALPVEYANLRFYGAGPEETYADRRHAKLGVWSTTAGDDCAPYLLPQETGNHEDVRWAEITDDSGHGVRVKRGAGAKPFAMSLLPYSSTMLEEALHQDELPKPRHMFLRLLAAQMGVGGDDSWMSPVHEQYQLPADQPLSLNVQLKLF

SEQ ID NO: 2 - protein, 625 aa, Lactobacillus sakei
MQPNIQWLDTPAVFRVGQLPAHSDHRYYATLAEMAQQQSSFEQSLNGTWQFHYSVNAASRPKSFYELAFDAQDFEPITVPQHIELAGYEQLHYINTMYPWEGHYYRRPAFSTSDDKQHLGMFSEADYNPVGSYLHHFDLTPALRNQRVIIRFEGVEQAMYVWLNGQFIGYAEDSFTPSEFDLTPYLKETDNCLAVEVHKRSSAAFIEDQDFFRFFGIFRDVKLLAKPRTHLEDLWVIPEYDVVQQTGQVKLRLQFSGDENRVHLRIRDQHQIILTADLTSAAQVNGLYKMPELVQAWSNQTPNLYTLELEVVDQAGETIEISQQPFGFRKIEIKDKVMLLNGKRLVINGVNRHEWHPETGRTITAEDEAWDIACMQRNHINAVRTSHYPDRLSFYNGCDQAGIYMMAETNLESHGSWQKMGAVEPSWNVPGSYDEWEAATLDRARTNFETFKNHVSILFWSLGNESYAGSVLEKMNAYYKQQDPTRLVHYEGVFRAPEYKATISDVESRMYATPAEIKAYLDNAPQKPFILCEYMHDMGNSLGGMQSYIDLLSQYDMYQGGFIWDFIDQALLVTDPVTGQRELRYGGDFDDRPSDYEFSGDGLVFATRDEKPAMQEVRYYYGEHK

SEQ ID NO: 3 - protein, 330 aa, Lactobacillus sakei
MKNQQCRRLDTIMANTNKRLAVIFGDVTLGLKGPDFHYLFSYQTGGPESLRIQGKEWLYRSPKPTFWRATTDNDRGNQFPLKSGMWLAADQFIACQSITVAIDGQTIPLPIAPENNRYSGQETAQEVTVTYTYQTITTPQTTVEVSYTIQASGKIRVAVTYHGQAGLPSLPVFGLRFVMPTPATRFIYQGLSGETYPDRMAGGMAGEYEVTGLPVTPYLVPQDCGVHMATDWVTIYRQAVLDNRLREPVETGLKFKMVDQPFAFSCLPYTAEELENATHHSELPAPHRTVLSLLGAVRGVGGIDSWGSDVEAAYQIDATQDHHLEFEISF

SEQ ID NO: 4 - protein, 1049 aa, Bifidobacterium adolescentis
MADTAELAIVHATTASASWLTDPTVFAANRKPAHSSHRYVIGETSEPKQSLDGEWKVRIEQARNVDVESAPFAAVDFEDGDFGAIEVPGHLQMAGYLKNKYVNIQYPWDGHEDPQAPNIPENNHVAIYRRRFALDAQLARTLENDGTVSLTFHGAATAIYVWLDGTFVGYGEDGFTPSEFDVTEALRNGNGNAADSPEAEHTLTVACYEYSSASWLEDQDFWRLHGLFRTVELAAQPHTHVETVQLEADYTAADTAGTADTAELNAALTLRNPADAMTIESTLRDGDGNVVWESTQACNGEIALNSGKMTNIAPWSAESPTLYTLTVRVVGHDGAIIETVTQKIGFRTFRIENGIMTLNGKRIVFKGADRHEFDAKRGRAITREDMLSDVVFCKRHNINAIRTSHYPNQEYWYDLCDEYGLYLIDETNMETHGTWVANNVERPEDGIPGSRPEWEGACVDRINSMMRRDYNHPSVLIWSLGNESSAGEVFRAMYRHAHTIDPNRPVHYEGSVHMREFEDVTDIESRMYAHADEIERYLNDGSPAHTDGPKKPYISCEYMHAMGNSCGNMDEYTALERYPMYQGGFIWDFIDQAIETKLPDGTTRMCYGGDFGDRPSDYEFSGDGLLFADRTPSPKAQEVKQLYANVKIAVSVDEARITNDNLFVSTGDYRFVLRILADGKPVWSTTRRFDVAAGESASFEVDWPVDDYRSNAEELVLEVSQQLGNACDWAPAGYELAFGQCVVAGAKTTADAVDAAGAPADGTVTLGRWNAGVRGQGREALFSRTQGGMVSYTFGEREFVLRRPSITTFRPLTDNDRGAGHAFERAAWAVAGKYARCVDCAIANRGENAVEATYTYELAIPQRTKVTVRYVADTAGLVSLDVEYPGEKNGDLPTIPAFGIEWALPVEYANLRFYGAGPEETYADRRHAKLGVWSTTAGDDCAPYLLPQETGNHEDVRWAEITDDSGHGVRVKRGAGAKPFAMSLLPYSSTMLEEALHQDELPKPRHMFLRLLAAQMGVGGDDSWMSPVHEQYQLPADQPLSLNVQLKLF

SEQ ID NO: 5 - protein, 626 aa, Lactobacillus amylovorus
MKANIKWLDDPEVFRINQLPAHSDHPFYKDYREWQNHSSSFKQSLNGAWQFHFSKDPQSRPIDFYKRSFDSSSFDTIPVPSEIELNGYAQNQYTNILYPWESKIYRKPAYTLGRGIKDGDFSQGKDNTVGSYLKHFDLNPALAGHDIHIQFEGVERAMYVYLNGHFIGYAEDSFTPSEFDLTPYIQAKDNILAVEVFKHSTASWLEDQDMFRFSGIFRSVELLALPRTHLMDLDIKPTVVNDYHDGVFNAKLHFMGKTSGNVHVLIEDIDGKTLLNKKLPLKSTVEIENETFANVHLWDNHDPYLYQLIIEVHDQDGKLVELIPYQFGFRKIEITKDHVVLLNGKRLIINGVNRHEWDAKRGRSITLADMKQDIATFKHNNINAVRTCHYPNQIPWYYLCDQNGIYMMAENNLESHGTWQKLGQVEATSNVPGSIPEWREVVVDRARSNYETFKNHTAILFWSLGNESYAGSNIAAMNKLYKDHDSSRLTHYEGVFHAPEFKKEISDLESCMYLPPKEAEEYLQNPKKPLVECEYMHDMGTPDGGMGSYIKLIDKYPQYMGGFIWDFIDQALLVHDPVSGQDVLRYGGDFDDRHSDYEFSGDGLMFADRTPKPAMQEVRYYYGLHK

SEQ ID NO: 6 - protein, 316 aa, Lactobacillus amylovorus
MAYTNNLHVVYGEASLGVNGQDFAYLFSYERGGLESLKIKDKEWLYRTPTPTFWRATTDNDRGSGFNQKAAQWLGADMFTKCVGIHVQVDDHRFDELPVAPINNQFSNQEFAHEVKVAFDYETLTTPATKVKIIYNINDFGHMTITMHYFGKKGLPPLPVIGMRFIMPTKAKSFDYTGLSGETYPDRMAGAERGTFHIDGLPVTKYLVPQENGMHMQTNELVITRNSTQNNADKDGDFSLKITQTKQPFNFSLLPYTAEELENATHIEELPLARRSVLVIAGAVRGVGGIDSWGSDVEEQYHIDPEQDHEFSFTLN

SEQ ID NO: 7 - protein, 1052 aa, Bifidobacterium bifidum
MNTTDDQRKNGDPIVSPSIPTTAWLADPRVYAVHRLDAHSDHACWSRSPVDGESTDLRQSLDGEWRVRVETAPTGRFPDGTSDGPDWISDVSPLFAAPGFDDSSFSRVQVPSHLETAGLLAPQYVNVQYPWDGHEDPKAPAIPEHGHVAVYRREFDADGEVAQAVREGRPVTLTFQGAATAIYVWLNGSFIGYAEDSFTPSEFDVTDAIKVDGNVLAVACYEYSSASWLEDQDFWRLHGLFRSVELNARPAAHVADLHADADWDLATSRGSLSLDVLIDGAANAATADFALRDKNGTIVWRTATKADGTLHAEAEIDDAAPWSAERPDLYELSVTLLDADGKVLETARTRIGFRHVAIEDGILKLNGKRLVFRGVNRHEFDCRRGRAITEEDMLWDIRFMKRHNINAVRTSHYPNQSRWYELCDEYGIYLIDETNLETHGSWNSPGDIPVGTSVPGDDEAWLGACIDRLDSMILRDRNHPSVLVWSLGNESYAGEVLKAMSAHAHQLDPGRPVHYEGVNWNHAYDGISDFESRMYAKPAEIQDWLEHGDERGEASKPFVSCEYMHAMGNSCGGLSEFIDLERYERYSGGFIWDYIDQGLVQRLPDGSERLSVGGEWGDRPTDYEFVGNGIVFADRTPSPKAQEVKQLYSPVKLAPDGHGVTIENRNLFAGTDGYVFAARLLEDGHEIWHADYRFDVAAGDTQHHDIAFPDIDADGDTREVTYEVDLLLAEATAWAPAGYELAFGQLTGTLNPEQDITETSHDDDGRATRTLSRWNAGIRRDDEEILLSRTQGGIVSWKRDDREMVIRRPELVTFRPLTDNDRGNHSGFDRAAWFAAGRYAIVTETKIHESDDGLVAEYQYELADPNHTPVSVTYHVNSDMRMQLTVEYPGNATDMASLPAFGIEWELPGEYDRLRYYGPGPEETYRDRKQGGKLGIWDATAKASMAPYLMVQETGSHEDVRWLEATDIQGHGLRVTQRGDRHFTASLLPWNTYTIEAARRHEDLPKPRHNYLRLLAAQMGVGGDDSWGAPVHTAYQLPAGRPLTLDVNLELI

SEQ ID NO: 8 - protein, 1052 aa, Bifidobacterium bifidum
MNTTDDQRKNGDPIVSPSIPTTAWLADPRVYAVHRLDAHSDHACWSRSPVDGESTDLRQSLDGEWRVRVETAPTGRFPDGTSDGPDWISDVSPLFAAPGFDDSSFSRVQVPSHLETAGLLAPQYVNVQYPWDGHEDPKAPAIPEHGHVAVYRREFDADGEVAQAVREGRPVTLTFQGAATAIYVWLNGSFIGYAEDSFTPSEFDVTDAIKVDGNVLAVACYEYSSASWLEDQDFWRLHGLFRSVELNARPAAHVADLHADADWDLATSRGSLSLDVLIDGAANAATADFALWDKNGTIVWHIVTKADGTLHAEAEIDDAAPWSAERPDLYELSVTLLDADGKVLETARTRIGFRHVAIEDGILKLNGKRLVFRGVNRHEFDCRRGRAITEEDMLWDIRFMKRHNINAVRTSHYPNQSRWYELCDEYGIYLIDETNLETHGSWNSPGDIPVGTSVPGDDEAWLGACIDRLDSMILRDRNHPSVLVWSLGNESYAGEVLKAMSAHAHRLDPGRPVHYEGVNWNHAYDGISDFESRMYAKPAEIQDWLEHGDERGEASKPFVSCEYMHAMGNSCGGLSEFIDLERYERYSGGFIWDYIDQGLVQRLPDGSERLSVGGEWGDRPTDYEFVGNGIVFADRTPSPKAQEVKQLYSPVKLAPDGHGVTIENRNLFAGTDGYVFAARLLEDGHEIWHADYRFDVAAGDTQHHDIAFPDIDADGDTREVTYEVDLLLAEATAWAPAGYELAFGQLTGTLNPEQDITETSHDDDGRATRTLSRWNAGIRRDDKEILLSRTQGGIVSWKRDDREMVIRRPELVTFRPLTDNDRGNHSGFDRAAWFAAGRYAIVTETKIHESDDGLVAEYQYELADPNHTPVSVTYHVNSDMRMQLTVEYPGNATDMASLPAFGIEWELPGEYDRLRYYGPGPEETYRDRKQGGKLGIWDATAKASMAPYLMVQETGSHEDVRWLEATDIQGHGLRVTQRGDRHFTASLLPWNTYMIEAARRHEDLPEPRHNYLRLLAAQMGVGGDDSWGAPVHTAYQLPAGRPLTLDVNLELI

SEQ ID NO: 9 - protein, 1055 aa, Bifidobacterium breve
MTNSMQGKAKTIMTNLQSAQQFSQAWLTDPRVFAVNRLAAHSSHKFYDHSPQCGEAMDLKQSLDGQWRVQMLDLADLADNELAEAAFAQPGYDAAGFSPIEVPSALETKGFLNHQYVNQQYPWSGHESPVAPDVPKHNHVALYRHEFSLEPKAAAVLEANKTAADDAAKRRVTLTFQGAATAIVVWLNGAFIGYAEDSFTPSEFDVTDVLRDGVNTLAVACFEFSSASWLEDQDFWRLHGIFRSVELEAQPLVHVNDLRVLADYDHTTGEGSLDVVALLRNAGTAAAVAATVLDAAGNTVWHSKLTAGADAETLTVKANVGKVNPWSAEEPTLYTLQVVATDAAGQVIEAALQRIGFRHFAIEDGLMKLNGKRIVFKGVDRHEFDARTGRTIAEADMIEDIHSFKRLNINAVRTSHYPNETRWYELCDEYGIYVLDETNLETHGSWTDPGDVFQPARAIPGSKDEWRAACVDRTASMVRRDYNHPSVVIWSLGNEAFGGDVFYSMRDFVHENDPFRPVHYEGTFNDPEFSAATDIMSRMYAKPDEIVKLYLGEDGKKPYISCEYSHSMGNSTGGLHLYTELERYPLYQGGFIWDYVDQALWQDCGNGTERLAYGGDFEDRPNDYEFSGDGVMFADRTPSPKAQEVKQLYANVKLVPDESGVTITNDNLFISTASSLFTARVLVDGAERWHANYRFDVPAGETVREPIAFPKVTDLVALSGSAEVTYEVDQRLAEATDWAPAGYELTFGQYVAAVSFDDGAADAVVAGDAEVAADGFNAGIHTDFGEVLLSKTQGGMVSFKRDGREMVIRRPNLTTFRALTDNDRGNGSGFERAQWMAAGRYARVTGTSVEETADGKGLKATYSYELADAKHTPVTVHYEVDAALRVHLTVEYPGEADAATLPAFGLEWILPKQYDRLRFYGLGPEETYADRLHGAKLGVFSRTAAEDCAPYLLPQETGNHEQVRWAEITDEYGHGMRVTAAGGTRFATSLLPYSSLMFEDALHQNELPKPRHTFLRLLAAQMGVGGDDTWGAPVHDEFQVPADQPLKLDVTLELI

SEQ ID NO: 10 - protein, 689 aa, Bifidobacterium catenulatum
MTQRRSYRWPQPLAGQQARIWYGGDYNPDQWPEEVWDDDVRLMKKAGVNLVSVGIFSWAKIETSEGVYDFDWLDRIIDKLGEAGIAVDLASATASPPMWLTQAHPEVLWKDYRGDVCQPGARQHWRPTSPVFREYALKLCRAMAEHYKGNPYVVAWHVSNEYGCHNRFDYSEDAERAFRKWCEERYGTIDAVNDAWGTAFWAQRMNDFTEIVPPRFIGDGNFMNPGKLLDFKRFSSDALKAFYVAERDALAEITPDLPLTTNFMVSAAGSVLDYDDWGREVDFVSNDHYFIPGEAHLDELAFSASLVDGIARKDPWFLMEHSTSAVNWRPVNYRKEPGQLVRDSLAHVAMGADAVCYFQWRQSKAGAEKFHSAMVPHTGEDSAVFRDVCELGADLNTLADNGLLGTKLAKSKVAVVFDYESEWATEHTATPTQKVHHVDEPLQWFRALADHGVTADVVPVSSNWDEYEVVVLPSVYILSEETTRRVRDYVVNGGRLIVTYYTGLSDEKDHVWLGGYPGSIRDVVGVRVEEFMPMGDDFPGVPDCLGLSNGAVAHDIADVIGSVDGTATVLETFRDDPWTGMDGAPAIVANTFGEGRSVYVGARLGRDGIAKSLPEIFESLGMAETGENDSRVLRVEREGSDGSRFVFSFNRTHEAVQIPFEGKIVVSSFAEVSGENVSIKPNGVIVTKQ

SEQ ID NO: 11 - protein, 1023 aa, Bifidobacterium catenulatum
MANSNRVEHASETWLTDATVFEVNRTPAHSNHKCFTHDPQSGEHSDLTQSLDGEWRVEIVQASDIDFNEEPFVAENFDDSSFCRAQVPGHLQMAGLLKNKYVNIQYPWDGHENPLEPNVPENNHVALYRRKFVVSKRLADTKESEGSVSIVFHGMATAIYVWVNGLFAGYGEDGFTPNEFDITDLLHDGENVVAVACYEYSSASWLEDQDFWRLHGLFRSVELTAQPHVHVENMQLEADWDAESGTASLDAALSVRNASDAATISATLKDSEGNVVWEASTNADANTTFASGSLQGLEPWSAESPSLYELEVNVIDQAGNIVEAAVQKVGFRRFRIENGIMTLNGKRIVFKGADRHEFDAKRGRSITEQDMIDDVIFCKRHNINAIRTSHYPNQERWYDLCDEYGIYLIDETNLETHGSWCLPGDVVTAETAVPGSKAHWEGACVDRVNSMVRRDYNHPSVVIWSLGNESYTGDVFRAMYKHVHDIDPNRPVHYEGVTKNRDYDDVTDIETRMYEHADVVEEYLKNDPQKPYISCEYMHAMGNSVGNLDEYTALERYPHYQGGFIWDFIDQAIYATQPDGSTRLCYGGDFGDRPSDYEFSGNGLVFADRTPTPKAQEVKQLYSNVHIDVTDRSVSIKNDNLFISTGGYQFVLRILADGEPVWQSERRFDVPADSACTFDVEWPVDLYRANADELVLEVSQRLAEATDWAPAGYELAFGQTIVAGTKAAEDAALPADGIVTVGRWNAGVQGSGREILLSRTQGGLVSYTFDGHEFVLRRPAITTFRALTDNDRGAGHGFERAQWMVAGRYARCVDNVIEQVDEDTLKAVYTYELATPQCTKVTVGYTADTTGRLNLHVEYPGESGELPTIPAFGIEWTLPVQYSNLRFFGAGPEETYQDRKHAKLGVWSTDAFKDHAPYLMPQETGNHEEVRWAEITDENGHGLRVSRANGAAPFAVSLQPYSSFMIEEAQHQDELPAPKHMFLRVLAAQMGVGGDDSWMSPVHSQYHITADQPISLDVNLELI

SEQ ID NO: 12 - protein, 1008 aa, Lactobacillus delbrueckii
  source 1..1008: note = subspecies bulgaricus
MSNKLVKEKRVDQADLAWLTDPEVYEVNTIPPHSDHESFQSQEELEEGKSSLVQSLDGDWLIDYAENGQGPVNFYAEDFDDSNFKSVKVPGNLELQGFGQPQYVNVQYPWDGSEEIFPPQIPSKNPLASYVRYFDLDEAFWDKEVSLKFDGAATAIYVWLNGHFVGYGEDSFTPSEFMVTKFLKKENNRLAVALYKYSSASWLEDQDFWRMSGLFRSVTLQAKPRLHLEDLKLTASLTDNYQKGKLEVEANIAYRLPNASFKLEVRDSEGDLVAEKLGPIRSEQLEFTLADLPVAAWSAEKPNLYQVRLYLYQAGSLLEVSRQEVGFRNFELKDGIMYLNGQRIVFKGANRHEFDSKLGRAITEEDMIWDIKTMKRSNINAVRCSHYPNQSLFYRLCDKYGLYVIDEANLESHGTWEKVGGHEDPSFNVPGDDQHWLGASLSRVKNMMARDKNHASILIWSLGNESYAGTVFAQMADYVRKADPTRVQHYEGVTHNRKFDDATQIESRMYAPAKVIEEYLTNKPAKPFISVEYAHAMGNSVGDLAAYTALEKYPHYQGGFIWDWIDQGLEKDGHLLYGGDFDDRPTDYEFCGNGLVFADRTESPKLANVKALYANLKLEVKDGQLFLKNDNLFTNSSSYYFLTSLLVDGKLTYQSRPLTFGLEPGESGTFALPWPEVADEKGEVVYRVTAHLKEDLPWADEGFTVAEAEEVAQKLPEFKPEGRPDLVDSDYNLGLKGNNFQILFSKVKGWPVSLKYAGREYLKRLPEFTFWRALTDNDRGAGYGYDLARWENAGKYARLKDISCEVKEDSVLVKTAFTLPVALKGDLTVTYEVDGRGKIAVTADFPGAEEAGLLPAFGLNLALPKELTDYRYYGLGPNESYPDRLEGNYLGIYQGAVKKNFSPYLRPQETGNRSKVRWYQLFDEKGGLEFTANGADLNLSALPYSAAQIEAADHAFDLTNNYTWVRALSAQMGVGGDDSWGQKVHPEFCLDAQKARQLRLVIQPLLLK

SEQ ID NO: 13 - protein, 1007 aa, Lactobacillus delbrueckii
  source 1..1007: note = subspecies lactis
MSNKLVKEKRVDQADLAWLTDPEVYEVNTIPPHSDHESFQSQEELEEGKSSLVQSLDGNWLIDYAENGQGPINFYAEDFDDSNFKSVKVPGNLELQGFGQPQYVNIQYPWDGSEEIFPPQVPSKNPLASYVRYFDLDEALWDKEVSLKFAGAATAIYVWLNGHFVGYGEDSFTPSEFMVTKFLKKEGNRLAVALYKYSSASWLEDQDFWRLSGLFRSVTLEAKPLLHLEDLKLTASLTDNYQKGKLEVEANIAYRLPNASFKLEVRDSEGDLVAEKVGPIRSEKLGFSLADLPVAAWSAEKPNLYQVRLYLYQAGSLLEVSRQEVGFRNFELKDGIMYLNGQRIVFKGVNRHEFDSKLGRAITEADMIWDIKTMKQSNINAVRCSHYPNQSLFYRLCDKYGLYVIDEANLESHGTWEKVGHEDPSFNVPGDDQHWLGASLSRVKNMMARDKNHASILIWSLGNESYAGTVFAQMADYVRKADPTRVQHYEGVTHNRKFDDATQIESRMYAPAKEIEEYLTKKPAKPFISVEYAHAMGNSVGDLAAYTALEKYPHYQGGFIWDWIDQGLEKDGHLLYGGDFDDRPTDYEFCGDGLVFADRTTSPKLANVKALYSNLKLEVKDGQLFIKNDNLFTNSSAYYFLASLLVDGKLTYQSQPLTFGLEPGESGTFVLPWPEVEDEKGEIVYQVTAHLKEDLPWADEGFTVAEAEEAVTKLPEFYPAGRPELVDSDFNLGLKGNGFRILFSKAKGWPVSIKYAGREYLKRLPEFTFWRALTDNDRGAGYGYDLAKWENAGKYARLQDISYEIKENSALVKTTFTLPVALKGDLTITYEVDSLGKIAVTANFPGAVENGLLPAFGLNFALPKELSDYRYYGLGPNESYADRLEGSYLGIYQGAVEKNFTPYLRPQEAGNRSKVRYYQLFDEEGGLEFTANGADLNLSALPYSAAQIEAADHAFELTNNYTWVRALAAQMGVGGDDSWGQKVHPEFCLDAQEARQLKLVIQPLLLK

SEQ ID NO: 14 - protein, 1008 aa, Lactobacillus delbrueckii
  source 1..1008: note = subspecies bulgaricus
MSNKLVKEKRVDQADLAWLTDPEVYEVNTIPPHFDHESFQSQEELEEGKSSLVQSLDGDWLIDYAENGQGPVNFYAEDFDDSNFKSVKVPGNLELQGFGQPQYVNVQYPWDGSEEIFPPQIPSKNPLASYVRYFDLDEAFWDKEVSLKFDGAATAIYVWLNGHFVGYGEDSFTPSEFMVTKFLKKENNRLAVALYKYSSASWLEDQDFWRMSGLFRSVTLQAKPRLHLEDLKLTASLTDNYQKGKLEVEANIAYRLPNASFKLEVRDSEGDLVAEKLGPIRSEQLEFTLADLPVAAWSAEKPNLYQVRLYLYQAGSLLEVSRQEVGFRNFELKDGIMYLNGQRIVFKGANRHEFDSKLGRAITEEDMIWDIKTMKRSNINAVRCSHYPNQSLFYRLCDKYGLYVIDEANLESHGTWEKVGGHEDPSFNVPGDDQHWLGASLSRVKNMMARDKNHASILIWSLGNESYAGTVFAQMADYVRKADPTRVQHYEGVTHNRKFDDATQIESRMYAPAKVIEEYLTNKPAKPFISVEYAHAMGNSVGDLAAYTALEKYPHYQGGFIWDWIDQGLEKDGHLLYGGDFDDRPTDYEFCGNGLVFADRTESPKLANVKALYANLKLEVKDGQLFLKNDNLFTNSSSYYFLTSLLVDGKLTYQSRPLTFGLEPGESGTFALPWPEVADEKGEVVYRVTAHLKEDLPWADEGFTVAEAEEVAQKLPEFKPEGRPDLVDSDYNLGLKGNNFQILFSKVKGWPVSLKYAGREYLKRLPEFTFWRALTDNDRGAGYGYDLARWENAGKYARLKDISCEVKEDSVLVKTAFTLPVALKGDLTVTYEVDGRGKIAVTADFPGAEEAGLLPAFGLNLALPKELTDYRYYGLGPNESYPDRLEGNYLGIYQGAVKKNFSPYLRPQETGNRSKVRWYQLFDEKGGLEFTANGADLNLSALPYSAAQIEAADHAFELTNNYTWVRALSAQMGVGGDDSWGQKVHPEFCLDAQKARQLRLVIQPLLLK

SEQ ID NO: 15 - protein, 1008 aa, Lactobacillus delbrueckii
  source 1..1008: note = subspecies bulgaricus
MSNKLVKEKRVDQADLAWLTDPEVYEVNTIPPHSDHESFQSQEELEEGKSSLVQSLDGDWLIDYAENGQGPVNFYAEDFDDSNFKSVKVPGNLELQGFGQPQYVNVQYPWDGSEEIFPPQIPSKNPLASYVRYFDLDEAFWDKEVSLKFDGAATAIYVWLNGHFVGYGEDSFTPSEFMVTKFLKKENNRLAVALYKYSSASWLEDQDFWRMSGLFRSVTLQAKPRLHLEDLKLTASLTDNYQKGKLEVEANIAYRLPNASFKLEVRDSEGDLVAEKLGPIRSEQLEFTLADLPVAAWSAEKPNLYQVRLYLYQAGSLLEVSRQEVGFRNFELKDGIMYLNGQRIVFKGANRHEFDSKLGRAITEEDMIWDIKTMKRSNINAVRCSHYPNQSLFYRLCDKYGLYVIDEANLESHGTWEKVGGHEDPSFNVPGDDQHWLGASLSRVKNMMARDKNHASILIWSLGNESYAGTVFAQMADYVRKADPTRVQHYEGVTHNRKFDDATQIESRMYAPAKVIEEYLTNKPAKPFISVEYAHAMGNSVGDLAAYTALEKYPHYQGGFIWDWIDQGLEKDGHLLYGGDFDDRPTDYEFCGNGLVFADRTESPKLANVKALYANLKLEVKDGQLFLKNDNLFTNSSSYYFLTSLLVDGKLTYQSRPLTFGLEPGESGTFALPWPEVADEKGEVVYRVTAHLKEDLPWADEGFTVAEAEEVAQKLPEFKPEGRPDLVDSDYNLGLKGNNFQILFSKVKGWPVSLKYAGREYLKRLPEFTFWRALTDNDRGAGYGYDLARWENAGKYARLKDISCEVKEDSVLVKTAFTLPVALKGDLTVTYEVDGRGKIAVTADFPGAEEAGLLPAFGLNLALPKELTDYRYYGLGPNESYPDRLEGNYLGIYQGAVKKNFSPYLRPQETGNRSKVRWYQLFDEKGGLEFTANGADLNLSALPYSAAQIEAADHAFELTNNYTWVRALSAQMGVGGDDSWGQKVHPEFCLDAQKARQLRLVIQPLLLK

SEQ ID NO: 16 - protein, 1008 aa, Lactobacillus delbrueckii
  source 1..1008: note = subspecies lactis
MSNKLVKEKRVDQADLAWLTDPEVYEVNTIPPHSDHESFQSQEELEEGKSSLVQSLDGDWLIDYAENGQGPVNFYAEDFDDSNFKSVKVPGNLELQGFGQPQYVNIQYPWDGSEEIFPPQVPSKNPLASYVRYFDLDEAFWDKEVSLKFAGAATAIYVWLNGHFVGYGEDSFTPSEFMVTKFLKKENNRLAVALYKYSSASWLEDQDFWRLSGLFRSVTLQAKPLLHLEDLKLTASLTDNYQKGKLEVEANIAYRLPNASFKLEVRDSEGDLVAEKLGPIRSEQLEFTLADLPVAAWSAEKPNLYQVRLYLYQAGSLLEVSRQEVGFRNFELKDGIMYLNGQRIVFKGVNRHEFDSKLGRAITEEDMIWDIKTMKRSNINAVRCSHYPNQSLFYRLCDKYGLYVIDEANLESHGTWEKVGHEDPSFNVPGDDQHWLGASLSRVKNMMARDKNHASILIWSLGNESYAGTVFAQMADYVRKADPTRVQHYEGVTHNRKFDDATQIESRMYAPAKEIEEYLTKKPAKPFISVEYAHAMGNSVGDLAAYTALEKYPHYQGGFIWDWIDQGLEKDGHLLYGGGDFDDRPTDYEFCGNGLVFADRTTSPKLANVKALYSNLKLEVKDGQLFLKNDNLFTNSSAYYFLTSLLVDGKLTYQSQPLTFGLEPGESGTFVLPWPEVEDEKGEIVYQVTAHLKEDLPWADEGFTVAEAEEAVTKLPEFYPAGRPELVDSDFNLGLKGNGFRILFSKAKGWPVSIKYAGREYLKRLPEFTFWRALTDNDRGAGYGYDLAKWENAGKYARLQDISYEIKENSVLVKTAFTLPVALKGDLTITYEVDSLGKIAVTANFPGAVENGLLPAFGLNFALPKELSDYRYYGLGPNESYADRLEGSYLGIYQGAVEKNFTPYLRPQEAGNRSKVRYYQLFDEESGLEFTANGADLNLSALPYSAAQIEAADHAFELSNNYTWVRALAAQMGVGGDDSWGQKVHPEFCLDAQEARQLKLVIQPLLLK

SEQ ID NO: 17 - protein, 1008 aa, Lactobacillus delbrueckii
  source 1..1008: note = subspecies bulgaricus
MSNKLVKEKRVDQADLAWLTDPEVYEVNTIPPHSDHESFQSQEELEEGKSSLVQSLDGDWLIDYAENGQGPVNFYAEDFDDSNFKSVKVPGNLELQGFGQPQYVNVQYPWDGSEEIFPPQIPSKNPLASYVRYFDLDEAFWDKEVSLKFDGAATAIYVWLNGHFVGYGEDSFTPSEFMVTKFLKKENNRLAVALYKYSSASWLEDQDFWRMSGLFRSVTLQAKPRLHLEDLKLTASLTDNYQKGKLEVEANIAYRLPNASFKLEVRDSEGDLVAEKLGPIRSEQLEFTLADLPVAAWSAEKPNLYQVRLYLYQAGSLLEVSRQEVGFRNFELKDGIMYLNGQRIVFKGVNRHEFDSKLGRAITEEDMIWDIKTIKRSNINAVRCSHYPNQSLFYRLCDKYGLYVIDEANLESHGTWEKVGGHEDPSFNVPGDDQHWLGASLSRVKNMMARDKNHASILIWSLGNESYAGTVFAQMADYVRKADPTRVQHYEGVTHNRKFDDATQIESRMYAPAKVIEEYLTNKPAKPFISVEYAHAMGNSVGDLAAYTALEKYPHYQGGFIWDWIDQGLEKDGHLLYGGDFDDRPTDYEFCGNGLVFADRTESPKLANVKALYANLKLEVKDGQLFLKNDNLFTNSSSYYFLTSLLVDGKLTYQSRPLTFGLEPGESGTFALPWPEVADEKGEVVYRVTAHLKEDLPWADEGFTVAEAEEVAQKLPEFKPEGRPDLVDSDYNLGLKGNNFQILFSKVKGWPVSLKYAGREYLKRLPEFTFWRALTDNDRGAGYGYDLARWENAGKYARLKDISCEVKEDSVLVKTAFTLPVALKGDLTVTYEVDGRGKIAVTADFPGAEEAGLLPAFGLNLALPKELTDYRYYGLGPNESYPDRLEGNYLGIYQGAVKKNFSPYLRPQETGNRSKVRWYQLFDEKGGLEFTANGADLNLSALPYSAAQIEAADHAFDLTNNYTWVRALSAQMGVGGDDSWGQKVHPEFCLDAQKARQLRLVIQPLLLK

SEQ ID NO: 18 - protein, 1008 aa, Lactobacillus delbrueckii
  source 1..1008: note = subspecies bulgaricus
MSNKLVKEKRVDQADLAWLTDPEVYEVNTIPPHSDHESFQSQEELEEGKSSLVQSLDGDWLIDYAENGQGPVNFYAEDFDDSNFKSVKVPGNLELQGFGQPQYVNVQYPWDGSEEIFPPQIPSKNPLASYVRYFDLDEAFWDKEVSLKFDGAATAIYVWLNGHFVGYGEDSFTPSEFMVTKFLKKENNRLAVALYKYSSASWLEDQDFWRMSGLFRSVTLQAKPRLHLEDLKLTASLTDNYQKGKLEVEANIAYRLPNASFKLEVRDSEGDLVAEKLGPIGSEQLEFTLADLPVAAWSAEKPNLYQVRLYLYQAGSLLEVSRQEVGFRNFELKDGIMYLNGQRIVFKGVNRHEFDSKLGRAITEEDMIWDIKTIKRSNINAVRCSHYPNQSLFYRLCDKYGLYVIDEANLESHGTWEKVGGHEDPSFNVPGDDQHWLGASLSRVKNMMARDKNHASILIWSLGNESYAGTVFAQMADYVRKADPTRVQHYEGVTHNRKFDDATQIESRMYAPAKVIEEYLTNKPAKPFISVEYAHAMGNSVGDLAAYTALEKYPHYQGGFIWDWIDQGLEKDGHLLYGGDFDDRPTDYEFCGNGLVFADRTESPKLANVKALYANLKLEVKDGQLFLKNDNLFTNSSSYYFLTSLLVDGKLTYQSRPLTFGLEPGESGTFALPWPEVADEKGEVVYRVTAHLKEDLPWADEGFTVAEAEEVAQKLPEFKPEGRPDLVDSDYNLGLKGNNFQILFSKVKGWPVSLKYAGREYLKRLPEFTFWRALTDNDRGAGYGYDLARWENAGKYARLKDISCEVKEDSVLVKTAFTLPVALKGDLTVTYEVDGRGKIAVTADFPGAEEAGLLPAFGLNLALPKELTDYRYYGLGPNESYPDRLEGNYLGIYQGAVKKNFSPYLRPQETGNRSKVRWYQLFDEKGGLEFTANGADLNLSALPYSAAQIEAADHAFELTNNYTWVRALSAQMGVGGDDSWGQKVHPEFCLDAQKARQLRLVIQPLLLK

SEQ ID NO: 19 - protein, 1007 aa, Lactobacillus delbrueckii
  source 1..1007: note = subspecies lactis
MSNKLVKEKRVDQADLAWLTDPEVYEVNTIPPHSDHESFQSQEELEEGKSSLVQSLDGNWLIDYAENGQGPINFYAEDFDDSNFKSVKVPGNLELQGFGQPQYVNIQYPWDGSEEIFPPQVPSKNPLASYVRYFDLDEALWDKEVSLKFAGAATAIYVWLNGHFVGYGEDSFTPSEFMVTKFLKKEGNRLAVALYKYSSASWLEDQDFWRLSGLFRSVTLEAKPLLHLEDLKLTASLTDNYQKGKLEVEANIAYRLPNASFKLEVRDSEGDLVAEKVGPIRSEKLDFSLADLPVAAWSAEKPNLYQVRLYLYQAGSLLEVSRQEVGFRNFELKDGIMYLNGQRIVFKGVNRHEFDSKLGRAITEADMIWDIKTMKQSNINAVRCSHYPNQSLFYRLCDKYGLYVIDEANLESHGTWEKVGHEDPSFNVPGDDQHWLGASLSRVKNMMARDKNHASILIWSLGNESYAGTVFAQMADYVRKADPTRVQHYEGVTHNRKFDDATQIESRMYAPAKEIEEYLTKKPAKPFISVEYAHAMGNSVGDLAAYTALEKYPHYQGGFIWDWIDQGLEKDGHLLYGGDFDDRPTDYEFCGDGLVFADRTTSPKLANVKALYSNLKLEVKDGQLFIKNDNLFTNSSAYYFLTSLLVDGKLTYQSQPLTFGLEPGESGTFALPWPEVEDEKGEIVYQVTAHLKEDLPWADEGFTVAEAEEAVTKLPEFYPAGRPELVDSDFNLGLKGNGFRILFSKAKGWPVSIKYAGREYLKRLPEFTFWRALTDNDRGAGYGYDLAKWENAGKYARLQDISYEIKENSALVKTAFTLPVALKGDLTITYEVDSLGKIAVTANFPGAVENGLLPAFGLNFALPKELSDYRYYGLGPNESYADRLEGSYLGIYQGMVEKNFTPYLRPQEAGNRSKVRYYQLFDEEGGLEFTANGADLNLSALPYSAAQIEAADHAFELTNNYTWVRALAAQMGVGGDDSWGQKVHPEFCLDAQEARQLKLVIQPLLLK

SEQ ID NO: 20 - protein, 628 aa, Lactobacillus helveticus
MQANINWLDNPEVFRVNQLPAHSDHPFFRDYREWQKQHSSYQQSLNGKWKFHFSANPMDRPQDFYQRDFDSSNFDSIPVPSEIELSNYTQNQYINVLFPWEGKIFRRPAYALDPNDHEEGSFSKGADNTVGSYLKRFDLSSALIGKDVHIKFEGVEQAMYVWLNGHFVGYAEDSFTPSEFDLTPYIQDKDNLLAVEVFKHSTASWLEDQDMFRFSGIFRSVELLGIPATHLMDMDLKPRVADNYQDGIFNLKLHFIGKKAGSFHLLVKDIKGHTLLEKNEDIKENVQINNEKFENVHLWNNHDPYLYQLLIEVYDEQQNLLELIPFQFGFRRIEISPEKVVLLNGKRLIINGVNRHEWDAKRGRSITMSDMTTDINTFKENNINAVRTCHYPNQIPWYYLCDQNGIYVMAENNLESHGTWQKMGEIEPSDNVPGSIPQWKEAVIDRARNNYETFKNHTSILFWSLGNESYAGDNIIAMNEFYKSHDDTRLVHYEGVVHRPELKDKISDVESCMYLPPKKVEEYLQNDPPKPFMECEYMHDMGNSDGGMGSYIKLLDKYPQYFGGFIWDFIDQALLVHDEISGHDVLRYGGDFDDRHSDYEFSGDGLMFADRTPKPAMQEVRYYYGLHK

SEQ ID NO: 21 - protein, 318 aa, Lactobacillus helveticus
MDYTNNQLHIIYGDATFGVNGKDFQYIFSYERGGLESLKVHGKEWLYRVPTPTFWRATTDNDRGSGFNLKAAQWLGADMFTKCTDIHLKVDRHDFAELPIAPFNNKFSNHEYAKSAEISFTYQTLTTPATNAKIIYNIDDVGHIKVTMRYYGKKGLPPLPVIGIRLIMPTAATGFDYEGLSGETYPDRMAGAKEGKFHIDGLPVTEYLVPQENGMHMQTKKLTINRETTQNNVDRTNEKFSLSIQQAEKPFNFSCLPYTAEELENATHIEELPLVRRTVLVIAGAVRGVGGIDSWGTDVESAYHINPELDHEFSFILN

SEQ ID NO: 22 - protein, 1023 aa, Bifidobacterium longum
MTDVTHVDRASQAWLTDPTVFEVNRTPAHSSHKWYARDPQSGQWSDLKQSLDGEWRVEVVQAADINLEEEPATAESFDDSSFERIQVPGHLQTAGLMNHKYVNVQYPWDGHENPLEPNIPENNHVALYRRKFTVSAPVANAKQAGGSVSIVFHGMATAIYVWVNGAFVGYGEDGFTPNEFDITELLHDGENVVAVACYEYSSASWLEDQDFWRLHGLFRSVELAARPHVHIENTQIEADWDPEAGTASLDAALTVLNAADAATVRATLKDADGNTVWQTTGDAEAQTAISSGPLQGIAPWSAESPTLYELDVDVIDQAGDVIECTSQKVGFRRFRIEDGILTINGKRIVFKGADRHEFDAEQGRAITEQDMIDDVVFCKRHNINSIRTSHYPNQERWYELCDEYGIYLIDEANLEAHGSWSLPGDVLTEDTIVPGSKREWEGACVDRVNSMMRRDYNHPSVLIWSLGNESYVGDVFRAMYKHVHDIDPNRPVHYEGVTHNRDYDDVTDIETRMYSHADEIEKYLKDDPKKPYLSCEYMHAMGNSVGNMDEYTALERYPKYQGGFIWDFIDQAIYATQPDGTRSLRYGGDFGDRPSDYEFSGDGLLFANRKPSPKAQEVKQLYSNVHIDVTKDSVSVKNDNLFTATGDYVFVLSVLADGKPVWQSTRRFDVPAGETRTFDVAWPVAAYRADARELVLQVSQRLAKATDWAESGYELAFGQTVVPADATATPDTKPADGTITVGRWNAGVRGAGREVLLSRTQGGMVSYTFAGNEFVLRRPAITTFRPLTDNDRGAGHGFERVQWLGAGRYARCVDNVLEQIDDSTLKGTYTYELATAQRTKVTVSYTAHTDGRVNLHVEYPGEQGDLPTIPAFGIEWTLPVQYTNLRFFGTGPAETYLDRKHAKLGVWSTNAFADHAPYLMPQETGNHEDVRWAEITDDHGHGMRVSRADGAAPFAVSLLPYSSFMLEEAQHQDELPKPKHMFLRVLAAQMGVGGDDSWMSPVHPQYHIPADKPISLDVDLELI

SEQ ID NO: 23 - protein, 628 aa, Lactobacillus reuteri
MDADIKWLDEPETFRVNQLPAHSDHYYYGNYDEWRHNNSRFAQNLDGQWQFNFAENLRERENDFYKMDYDSSSFGTIEVPSEIELNNYAQNNYINTLIPWEGKIYRRPAYTLSPDDAQEGSFSDGDDNTIGEYLKHFDLDPSLRGKQVRIRFDGVERAMYVWLNGHFIGYAEDSFTPSEFDLTPYIQDEGNVLAVEVFKHSTASWIEDQDMFRFSGIFRSVNLLAQPLVHVEDLNIRPIVTDNYQDGIFNVELQLHGEKTGNVNVRVIDNDGNTLVNETHPVDSTVKVQDQFLENVHLWDNHDPYLYQLLIEIRDDEGNLVELVPYRFGFRRIEINKDHVVLLNGQRLIINGVNRHEWDARRGRAITMDDMTSDIHTFKENNINAVRTCHYPDQIPWYYLCDDNGIYMMAENNLESHATWQKMGAIEPSYNVPGSVPQWRDVVVDRARTNYETFKNHPSILFWSLGNESYAGDNIVKMNEFYKKHDDSRLVHYEGVCHTPEYRDRISDVESWMYLPPKEVEEYLKNNPDKPFMECEYMHDMGNSDGGMGSYISLLDKYPQYFGGFIWDFIDQALLVKDPVSGQEVMRYGGDFDDRHSDYEFSGDGLMFADRTPKPAMQEVRYYYGLHK

SEQ ID NO: 24 - protein, 319 aa, Lactobacillus reuteri
MAYTNKLRVIYGDATLGLSGDGFHYIFSYERGGLESLKLNGKEWLYREPMPTFWRATTDNDRGSGFNIRSAQWLAADTFHKCVGIDLTVDNQHFAELPIAPITNEFSDPVSAESVKIKYTFATLTVPATQVTVIYEVNGQGEIKVTMHYYGHEDLPGLPVVGMRFIMPTVATGFDYQGLSGETYPDRMAGATEGTFHVDGLPVTKYLVPQENGMHMATHALTITRDSTQNNADHSREPFSLTVKQDAQPFAFSCLPYTAEELENATHIEELPLARRTVLVVAGAVRGVGGIDSWGADVEEQYHIPADRDVEFSFVLNAK

SEQ ID NO: 25 - protein, 1007 aa, Lactobacillus delbrueckii
  source 1..1007: note = subspecies lactis
MSNKLVKEKRVDQADLAWLTDPEVYEVNTIPPHSDHESFQSQEELEEGKSSLVQSLDGNWLIDYAENGQGPINFYAEDFDDSNFKSVKVPGNLELQGFGQPQYVNIQYPWDGSEEIFPPQVPSKIPLASYVRYFDLDEALWDKEVSLKFAGAATAIYVWLNGHFVGYGEDSFTPSEFMVTKFLKKEGNRLAVALYKYSSASWLEDQDFWRLSGLFRSVTLEAKPLLHLEDLKLTASLTDNYQKGKLEVEANIAYRLPNASFKLEVRDSEGDLVAEKVGPIRSEKLDFSLADLPVAAWSAEKPNLYQVRLYLYQAGSLLEVSRQEVGFRNFELKDGIMYLNGQRIVFKGVNRHEFDSKLGRAITEADMIWDIKTMKQSNINAVRCSHYPNQSLFYRLCDKYGLYVIDEANLESHGTWEKVGHEDPSFNVPGDDQHWLGASLSRVKNMMARDKNHASILIWSLGNESYAGTVFAQMADYVRKADPTRVQHYEGVTHNRKFDDATQIESRMYAPAKEIEEYLTKKPAKPFISVEYAHAMGNSVGDLAAYTALEKYPHYQGGFIWDWIDQGLEKDGHLLYGGDFDDRPTDYEFCGDGLVFADRTTSPKLANVKALYSNLKLEVKDGQLFIKNDNLFTNSSAYYFLTSLLVDGKLTYQSQPLTFGLEPGESGTFALPWPEVEDEKGEIVYQVTAHLKEDLPWADEGFTVAEAEEAVTKLPEFYPAGRPELVDSDFNLGLKGNGFRILFSKAKGWPVSIKYAGREYLKRLPEFTFWRALTDNDRGAGYGYDLAKWENAGKYARLQDISYEIKENSALVKTTFTLPVALKGDLTITYEVDSLGKIAVTANFPGAVENGLLPAFGLNFALPKELSDYRYYGLGPNESYADRLEGSYLGIYQGMVEKNFTPYLRPQEAGNRSKVRYYQLFDEEGGLEFTANGADLNLSALPYSAAQIEAADHAFELTNNYTWVRALAAQMGVGGDDSWGQKVHPEFCLDAQEARQLKLVIQPLLLK

SEQ ID NO: 26 - protein, 628 aa, Lactobacillus helveticus
MQANINWLDNPEVFRVNQLPAHSDHPFFRDYREWQKQHSSYQQSLNGKWKFHFSANPMDRPQDFYQRDFDSSNFDSIPVPSEIELSNYTQNQYINVLFPWEGKIFRRPAYALDPNDHEEGSFSKGADNTVGSYLKRFDLSSALIGKDVHIKFEGVEQAMYVWLNGHFVGYAEDSFTPSEFDLTPYIQEKDNLLAVEVFKHSTASWLEDQDMFRFSGIFRSVELLGIPATHLMDMDLKPRVADNYQDGIFNLKLHFIGKKAGSFHLLVKDIKGHTLLEKNEDIKENVQINNEKFENVHLWNNHDPYLYQLLIEVYDEQQNLLELIPFQFGFRRIEISPEKVVLLNGKRLIINGVNRHEWDAKRGRSITMSDMTTDINTFKENNINAVRTCHYPNQIPWYYLCDQNGIYVMAENNLESHGTWQKMGEIEPSDNVPGSIPQWKEAVIDRARNNYETFKNHTSILFWSLGNESYAGDNIIAMNEFYKSHDDTRLVHYEGVVHRPELKDKISDVESCMYLPPKKVEEYLQNDPPKPFMECEYMHDMGNSNGGMDSYIKLLDKYPQYFGGFIWDFIDQALLVHDEISGHDVLRYGGDFDDRHSDYEFSGDGLMFADRKPKPAMQEVRYYYGLHK

SEQ ID NO: 27 - protein, 318 aa, Lactobacillus helveticus
MDYTNNQLHIIYGDATFGVNGKDFQYIFSYERGGLESLKVHGKEWLYRVPTPTFWRATTDNDRGSGFNLKAAQWLGADMFTKCTDIHLKVDRHDFAELPIAPFNNKFSNHEYAKSAEISFTYQTLTTPATNAKIIYNIDDGGHIKVTMRYYGKKGLPPLPVIGIRLIMPTAATGFDYEGLSGETYPDRMAGAKEGKFHIDGLPVTEYLVPQENGMHMQTKKLTINRETTQNNVDRTNEKFSLSIQQAEKPFNFSCLPYTAEELENATHIEELPLVRRTVLVIAGAVRGVGGIDSWGTDVESAYHINPDLDHEFSFILN

SEQ ID NO: 28 - protein, 626 aa, Lactobacillus crispatus
MKANIKWLDDPEVFRINQLPAHSDHPFYKDYREWQKHSSSFKQSLNGAWQFHFSKDPQSRPIDFYKLSFDSSSFDTIPVPSEIELNGYAQNQYTNILYPWESKIYRKPAYTLGRGIKDGDFSQGKDNTVGSYLKHFDLNPALAGHDIHIQFEGVERAMYVYLNGHFIGYAEDSFTPSEFDLTPYIQAKDNILAVEVFKHSTASWLEDQDMFRFSGIFRSVELLALPRTHLMDLDIKPTVVNDYHDGVFNAKLHFMGKTSGNVHVLIEDIDGKTLLNKKLPLKSTVEIENETFANVHLWDNHDPYLYQLIIEVHDQDGKLVELIPYQFGFRKIEITKDHVVLLNGKRLIINGVNRHEWDAKRGRSITLADMKQDIATFKHNNINAVRTCHYPNQIPWYYLCDQNGIYMMAENNLESHGTWQKLGQVEATSNVPGSIPEWREVVVDRARSNYETFKNHTAILFWSLGNESYAGSNIAAMNKLYKDHDSSRLTHYEGVFHAPEFKKEISDLESCMYLPPKEAEEYLQNPKKPLVECEYMHDMGNSDGGIGSYIKLIDKYPQYMGGFIWDFIDQALLVHDPVSGQDVLRYGGDFDDRHSDYEFSGDGLMFADRTPKPAMQEVRYYYGLHK

SEQ ID NO: 29 - protein, 316 aa, Lactobacillus crispatus
MAYTNNLHVVYGEASLGVNGQDFAYLFSYERGVLESLKIKDKEWLYRTPTPTFWRATTDNDRGSGFNQKAAQWLGADMFTKCVGIHVQVDDHQFDELPIAPINNQFSNQEFAHEVKVAFDYETLTTPATKVKIIYNINDAGHMTITMHYFGKKGLPPLPVIGMRFIMPTKAKSFDYTGLSGETYPDRMAGAERGTFHIDGLPVTKYLVPQENGMHMQTNELVITRNSTQNNADKDGDFSLKITQTKQPFNFSLLPYTAEELENATHIEELPLARRSVLVIAGAVRGVGGIDSWGSDVEEQYHIDPEQDHEFSFTLN

SEQ ID NO: 30 - protein, 1025 aa, Streptococcus thermophilus
MNMTKIQTYLNDPKIVSVNTVDAHSDHKYFESLEEFSEGEMKLRQSLNGKWKIHYAQNTNQVLKDFYKTEFDETDLNFINVPGHLELQGFGSPQYVNTQYPWDGKEFLRPPQVPQESNAVASYVKHFTLNDALKDKKVFISFQGVATSIFVWVNGNFVGYSEDSFTPSEFEISDYLVEGDNKLAVAVYRYSTASWLEDQDFWRLYGIFRDVYLYAIPKVHVQDLFVKGDYDYQTKAGQLDIDLKTVGDYEDKKIKYVLSDYEGIVTEGDASVNGDGELSVSLENLKIKPWSAESPKLYDLILHVLDDDQVVEVVPVKVGFRRFEIKDKLMLLNGKRIVFKGVNRHEFNARTGRCITEEDMLWDIKVMKQHNINAVRTSHYPNQTRWYELCDEYGLYVIDEANLETHGTWQKLGLCEPSWNIPASEPEWLPACLDRANNMFQRDKNHASVIIWSCGNESYAGKDIADMADYFRSVDNTRPVHYEGVAWCREFDYITDIESRMYAKPADIEEYLTTGKLVDLSSVSDKHFASGNLTNKPQKPYISCEYMHTMGNSGGGLQLYTDLEKYPEYQGGFIWDFIDQAIYKTLPNGSEFLSYGGDWHDRPSDYEFCGNGIVFADRTLTPKLQTVKHLYSNIKIAVDEKSVTIKNDNLFEDLSAYTFLARVYEDGRKVSESEYHFDVKPGEEATFPVNFVVEASNSEQIYEVACVLREATKWAPKGHEIVRGQYVVEKISTETPVKAPLNVVEGDFNIGIQGQNFSILLSRAQNTLVSAKYNGVEFIEKGPKLSFTRAYTDNDRGAGYPFEMAGWKVAGNYSKVTDTQIQIEDDSVKVTYVHELPGLSDVEVKVTYQVDYKGRIFVTANYDGKAGLPNFPEFGLEFAIGSQFTNLSYYGYGAEESYRDKLPGAYLGRYETSVEKTFAPYLMPQESGNHYGTREFTVSDDNHNGLKFTALNKAFEFSALRNSTEQIENARHQYELQESDATWIKVLAAQMGVGGDDSWGAPVHDEFLLSSADSYQLSFMIEPLN

SEQ ID NO: 31 - protein, 1008 aa, Lactobacillus delbrueckii
  source 1..1008: note = subspecies indicus
MNNKLAQVKRVDQADLAWLTDPEIYEVNTIPPHSDHESFQSLEELEEGKSSLVQSLDGDWLIDYAENGEGPANFYEENFDDSSFKSVKVPGNLELQGFGQPQYVNVQYPWDGSDEIFPPMIPSKNPVASYVRYFDLEEAFWDKEVSLKFAGAATAIYVWLNGHFVGYGEDSFTPSEFMVTKFLKKEGNRLAVALYKYSSASWLEDQDFWRMSGLFRSVTLEAKPLLHLQDLKLTASLTNDYQKGSLQVEADIDYRLPNSSFKLELRDSAGELVAEKVGPIRSEKLDFSLADLPVAAWSAEEPNLYQVRLSLYQQGSLLEVSRQEVGFRNFELKDGIMYLNGKRIVFKGVNRHEFDSKLGRAITEADMIWDIKTMKQSNINAVRCSHYPNQSIFYHLCDKYGLYVIDEANLESHGTWEKVGGHEDPSFNVPGDDQRWLGASLSRVKNMMARDKNHASILIWSLGNESYAGKVFAQMADYVRQADPTRVQHYEGVTHNRKFDDATQIESRMYAPAKEIEEYLTKKPAKPFVSCEYAHAMGNSVGDLAAYTALEKYPHYQGGFIWDWIDQGLEKEGHLLYGGDFDDRPSDYEFCGDGLVFADRTTSPKLANVKALYSNLKLELKDGQLFLKNDNLFTNSSAYYFLTSLLVDGKLTYQSQPLTFALEPGESGTFALPWPEVEDEKGEIVYQVTAHLKEDLPWADEGFTVAEAEEAVTKLPEFYPAGRPELVDSDYNLGIKGNGFRILFSKAKGWPVSIKYAGREYLKRLPEFTFWRALTDNDRGAGYGYDLAKWENAGKYARLQDISYEIKENSVLVKTAFTLPVALKGDLTITYEVDSLGKIAVTANFPGAVENGLLPAFGLNFALPKELSDYRYYGLGPNESYADRLEGSYLGIYQGAVEKNFTPYLRPQEVGNRSKVRYYQLFDEEGGLEFTANGANLNLSALPYSAAQIEAADHAFELTNNYTWVRALAAQMGVGGDDSWGQKVHPEFCLDAQEARQLKLVIQPLFTE

SEQ ID NO: 32 - protein, 1049 aa, Bifidobacterium adolescentis
MADTAELAIVHATTASASWLTDPTVFAANRKPAHSSHRYVIGETSEPKQSLDGEWKVRIEQARNVDVESAPFAAVDFEDGDFGAIEVPGHLQMAGYLKNKYVNIQYPWDGHEDPQAPNIPENNHVAIYRRRFALDAQLARTLENDGTVSLTFHGAATAIYVWLDGTFVGYGEDGFTPSEFDVTEALRNGNGNAADSPEAEHTLTVACYEYSSASWLEDQDFWRLHGLFRTVELAAQPHTHVETVQLEADYTAADTAGTADTAELNAALTLRNPADAMTIESTLRDGDGNVVWESTQACNGEIALNSGKMTNIAPWSAESPTLYTLTVRVVGHDGAIIETVTQKIGFRTFRIENGIMTLNGKRIVFKGADRHEFDAKRGRAITREDMLSDVVFCKRHNINAIRTSHYPNQEYWYDLCDEYGLYLIDETNMETHGTWVANNVERPEDGIPGSRPEWEDACVDRINSMMRRDYNHPSVLIWSLGNESSAGEVFRAMYRHAHTIDPNRPVHYEGSVHMREFEDVTDIESRMYAHADEIERYLNDGSPAHTDGPKKPYISCEYMHAMGNSCGNMDEYTALERYPMYQGGFIWDFIDQAIETKLPDGTTRMCYGGDFGDRPSDYEFSGDGLLFADRTPSPKAQEVKQLYANVKIAVSVDEARITNDNLFVSTGDYRFVLRILADGKPVWSTTRRFDVAAGESASFEVDWPVDDYRSNAEELVLEVSQQLGNACDWAPAGYELAFGQCVVAGAKTTADAVDAAGAPADGTVTLGRWNAGVRGQGREALFSRTQGGMVSYTFGEREFVLRRPSITTFRPLTDNDRGAGHAFERAAWAVAGKYARCVDCAIANRGENAVEATYTYELAIPQRTKVTVRYVADTAGLVSLDVEYPGEKNGDLPTIPAFGIEWALPVEYANLRFYGAGPEETYADRRHAKLGVWSTTAGDDCAPYLLPQETGNHEDVRWAEITDDSGHGVRVKRGAGAKPFAMSLLPYSSTMLEEALHQDELPKPRHMFLRLLAAQMGVGGDDSWMSPVHEQYQLPADQPLSLNVQLKLF

SEQ ID NO: 33 - protein, 1023 aa, Bifidobacterium adolescentis
MANETRIEHASETWLADSTVFEVNRVPAHSDHKCYAHDSQTNEWSDLRQSLDGEWRVEVVQASDIEFNEEPFVRENFDDSAFERIQVPGHLQMAGLMNNKYVNIQYPWDGHENPAEPNIPENNHVALYRKTFTMANRLADTKNAGGTVSIVFHGMATAIYVWVNGMFVGYGEDGFTPNEFDITEMLHDGENVVAVACYEYSSASWLEDQDFWRLHGLFRSVELAAQPHVHIENMQIESDWDPESGSASLDAALTVRNAADAATISATLKDSDGNVVWETANCADPDTSISTGSLNGIRPWSAEDPVLYEFEVTVIDHAGNIAEVAVQKVGFRRFRIEDGIMTINGKRIVFKGADRHEFDPKRGRAITEQDMIDDVVFCKRHNLNAIRTSHYPNQERWYELCDEYGIYLIDETNLETHGSWCLPGDVLTEETAVPGSKAHWEGACVDRVNSMVRRDYNHPSVLIWSLGNESYTGDVFRAMYKRVHDIDPNRPVHYEGVTHNRDYNDVTDIETRMYAHADAIEEYLKNDPQKPYISCEYMHAMGNSCGNMDEYTALERYPKYQGGFIWDFIDQAIYATQPDGTTSLRYGGDFGDRPSDYEFSGNGLVFADRKPTPKAQEVKQLYSNVHIDVAEDSVTIKNDNLFTSTGEYTFVLRVLADGEPVWQSERRFDVPAGSTEKLDVDWPLDLYRDGASELVLEVSQRLAKATNWAVAGYELAFGQTVVAGSKKASAPVKPVDGIVTVGRWNVGVQGSGREVLLSRTQGGLVSYTFNNREFVLRRPAVTTFRALTDNDRGAGHGFERAQWLGAGRYARCIGNEIEQIDENTVKASYTYELATPQRTKVTVSYTADTTGRVNLHVEYPGEPGDLPTIPAFGIEWTLPVQYSNLRFFGAGPEETYQDRKHAKLGVWSTDAFKDHAPYLMPQETGNHEDVRWAEITDEKGHGLRISRAEGAEPFAMSLQPYSSFMLEEAQHQDELPAPKHMFLRVLAEQMGVGGDDSWMSPVHPQYHIPADQPISLDVDLDLI

SEQ ID NO: 34 - protein, 1305 aa, Bifidobacterium bifidum
MVEDATRSDSTTQMSSTPEVVYSSAVDSKQNRTSDFDANWKFMLSDSVQAQDPAFDDSAWQQVDLPHDYSITQKYSQSNEAESAYLPGGTGWYRKSFTIDRDLAGKRIAINFDGVYMNATVWFNGVKLGTHPYGYSPFSFDLTGNAKFGGENTIVVKVENRLPSSRWYSGSGIYRDVTLTVTDGVHVGNNGVAIKTPSLATQNGGNVTMNLTTKVANDTEAAANITLKQTVFPKGGKTDAAIGTVTTASKSIAAGASADVTSTITAASPKLWSIKNPNLYTVRTEVLNGDTVLDTYDTEYGFRWTGFDATSGFSLNGEKVKLKGVSMHHDQGSLGAVANRRAIERQVEILQKMGVNSIRTTHNPAAKALIDVCNEKGVLVVEEVFDMWNRSKNGNTEDYGKWFGQTIAGDNAVLGGDKDETWAKFDLTSTINRDRNAPSVIMWSLGNEMMEGISGSVSDFPATSAKLVAWTKAADSTRPMTYGDNKIKANWNESNTMGDNLTANGGVVGTNYSDGANYDKIRTTHPSWAIYGSETASAINSRGIYNRTTGGAQSSDKQLTSYDNSAVGWGAVASSAWYDVVQRDFVAGTYVWTGFDYLGEPTPWNGTGSGAVGSWPSPKNSYFGIVDTAGFPKDTYYFYQSQWNDDVHTLHILPAWNENVVAKGSGNKVPVVVYTDAAKVKLYFTPKGSTEKRLIGEKSFTKKTTAAGYTYQVYEGTDKDSTAHKNMYLTWNVPWAEGTISAEAYDENNRLIPEGSTEGNASVTTTGKAAKLKADADRKTITADGKDLSYIEVDVTDANGHIVPDAANRVTFDVKGAGKLVGVDNGSSPDHDSYQADNRKAFSGKVLAIVQSTKEAGEITVTAKADGLQSSTVKIATTAVPGTSTEKTVRSFYYSRNYYVKTGNKPILPSDVEVRYSDGTSDRQNVTWDAVSDDQIAKAGSFSVAGTVAGQKISVRVTMIDEIGALLNYSASTPVGTPAVLPGSRPAVLPDGTVTSANFAVHWTKPADTVYNTAGTVKVPGTATVFGKEFKVTATIRVQRSQVTIGSSVSGNALRLTQNIPADKQSDTLDAIKDGSTTVDANTGGGANPSAWTNWAYSKAGHNTAEITFEYATEQQLGQIVMYFFRDSNAVRFPDAGKTKIQISADGKNWTDLAATETIAAQESSDRVKPYTYDFAPVGATFVKVTVTNADTTTPSGVVCAGLTEIELKTATSKFVTNTSAALSSLTVNGTKVSDSVLAAGSYNTPAIIADVKAEGEGNASVTVLPAHDNVIRVITESEDHVTRKTFTINLGTEQEFPADSDERD

SEQ ID NO: 35 - protein, 1025 aa, Kluyveromyces lactis
MSCLIPENLRNPKKVHENRLPTRAYYYDQDIFESLNGPWAFALFDAPLDAPDAKNLDWETAKKWSTISVPSHWELQEDWKYGKPIYTNVQYPIPIDIPNPPTVNPTGVYARTFELDSKSIESFEHRLRFEGVDNCYELYVNGQYVGFNKGSRNGAEFDIQKYVSEGENLVVVKVFKWSDSTYIEDQDQWWLSGIYRDVSLLKLPKKAHIEDVRVTTTFVDSQYQDAELSVKVDVQGSSYDHINFTLYEPEDGSKVYDASSLLNEENGNTTFSTKEFISFSTKKNEETAFKINVKAPEHWTAENPTLYKYQLDLIGSDGSVIQSIKHHVGFRQVELKDGNITVNGKDILFRGVNRHDHHPRFGRAVPLDFVVRDLILMKKFNINAVRNSHYPNHPKVYDLFDKLGFWVIDEADLETHGVQEPFNRHTNLEAEYPDTKNKLYDVNAHYLSDNPEYEVAYLDRASQLVLRDVNHPSIIIWSLGNEACYGRNHKAMYKLIKQLDPTRLVHYEGDLNALSADIFSFMYPTFEIMERWRKNHTDENGKFEKPLILCEYGHAMGNGPGSLKEYQELFYKEKFYQGGFIWEWANHGIEFEDVSTADGKLHKAYAYGGDFKEEVHDGVFIMDGLCNSEHNPTPGLVEYKKVIEPVHIKIAHGSVTITNKHDFITTDHLLFIDKDTGKTIDVPSLKPEESVTIPSDTTYVVAVLKDDAGVLKAGHEIAWGQAELPLKVPDFVTETAEKAAKINDGKRYVSVESSGLHFILDKLLGKIESLKVKGKEISSKFEGSSITFWRPPTNNDEPRDFKNWKKYNIDLMKQNIHGVSVEKGSNGSLAVVTVNSRISPVVFYYGFETVQKYTIFANKINLNTSMKLTGEYQPPDFPRVGYEFWLGDSYESFEWLGRGPGESYPDKKESQRFGLYDSKDVEEFVYDYPQENGNHTDTHFLNIKFEGAGKLSIFQKEKPFNFKISDEYGVDEAAHACDVKRYGRHYLRLDHAIHGVGSEACGPAVLDQYRLKAQDFNFEFDLAFE

SEQ ID NO: 36 - DNA, 30 bp, synthetic construct
  source 1..30: mol_type = other DNA; note = Primer
  modified_base 9: mod_base = OTHER; note = misc_feature - deoxyuracil
attaaccatgcgacgcaacttcgaatggcc

SEQ ID NO: 37 - DNA, 31 bp, synthetic construct
  source 1..31: mol_type = other DNA; note = Primer
  modified_base 9: mod_base = OTHER; note = misc_feature - deoxyuracil
atcttctctttaccgccttaccacgagcacg

SEQ ID NO: 38 - DNA, 33 bp, synthetic construct
  source 1..33: mol_type = other DNA; note = Primer
  modified_base 9: mod_base = OTHER; note = misc_feature - deoxyuracil
agagaagattttcagcctgatacagattaaatc

SEQ ID NO: 39 - DNA, 33 bp, synthetic construct
  source 1..33: mol_type = other DNA; note = Primer
  modified_base 9: mod_base = OTHER; note = misc_feature - deoxyuracil
atggttaattcctcctgttagcccaaaaaacgg

SEQ ID NO: 40 - DNA, 22 bp, synthetic construct
  source 1..22: mol_type = other DNA; note = Primer
cggcgtcacactttgctatgcc

SEQ ID NO: 41 - DNA, 20 bp, synthetic construct
  source 1..20: mol_type = other DNA; note = Primer
ccgcgctactgccgccaggc